(19) United States
(12) Patent Application Publication
(10) Pub. No.: US 2021/0190799 A1
(43) Pub. Date: Jun. 24, 2021

(54) METHOD FOR DIAGNOSING LIVER DISEASES AND METHOD FOR SCREENING THERAPEUTIC AGENT FOR LIVER DISEASES USING CHANGES IN EXPRESSION OF TM4SF5 PROTEIN

(71) Applicant: SEOUL NATIONAL UNIVERSITY R&DB FOUNDATION, Seoul (KR)

(72) Inventors: Jung Weon Lee, Seoul (KR); JiHye Ryu, Gyeongsangnam-do (KR); Jae Woo Jung, Seoul (KR)

(73) Assignee: SEOUL NATIONAL UNIVERSITY R&DB FOUNDATION, Seoul (KR)

(21) Appl. No.: 16/758,016

(22) PCT Filed: Oct. 26, 2018
(86) PCT No.: PCT/KR2018/012860

§ 371 Date: Apr. 21, 2020

(30) Foreign Application Priority Data
Oct. 26, 2017 (KR) 10-2017-0140514

(51) Int. Cl. G01N 33/68; C12Q 1/6883; A01K 67/027

(52) U.S. Cl. CPC G01N 33/6893; A01K 2267/03; A01K 67/0275; C12Q 2600/158; C12Q 2600/106; G01N 2800/085; G01N 2440/14; G01N 2333/5412; G01N 2333/525; G01N 2333/78; G01N 2333/495; G01N 2333/8146; G01N 2333/523; A01K 2217/052; A01K 2227/105; C12Q 1/6883

(57) ABSTRACT

The present invention relates to a method for diagnosing obesity and liver diseases and method for screening a therapeutic agent for liver diseases using changes in the expression of TM4SF5 protein. In particular, the present invention may be usefully used for measuring changes in the expression of TM4SF5 protein in order to diagnose obesity and liver diseases or screen candidate preventive or therapeutic agents for obesity and liver diseases, by confirming that: in a transgenic mouse having over-expressed TM4SF5 protein, characteristics of fatty liver and hepatitis appear as a metabolic disorder occurs, an increase occurs in the expression of at least one mRNA or protein.

### BACKGROUND OF THE INVENTION

### 1. Field of the Invention

The present invention relates to a method for diagnosing liver diseases and a method for screening a therapeutic agent for liver diseases using the expression changes of TM4SF5 (transmembrane 4 L six family member 5) protein by confirming the expression changes of mRNAs and proteins of Srebp1 (Sterol regulatory element-binding protein 1), Srebp2 (Sterol regulatory element-binding protein 2), Fasn (Fatty acid synthase), CD36 (cluster of differentiation 36), Fabp1 (Fatty Acid-Binding Protein 1), Vldlr (very-low-density-lipoprotein receptor), Ldlr (low density lipoprotein receptor), ApoB100 (Apolipoprotein B 100), Pparα (Peroxisome proliferator-activated receptor alpha), Pparγ (Peroxisome Proliferator Activated Receptor Gamma), Leptin, Accα (acetyl-CoA carboxylase alpha), Accβ (acetyl-CoA carboxylase beta), collagen I, collagen type I alpha 1 chain, laminins, laminin α5, laminin γ2, laminin γ3, Socs1 (Suppressor of cytokine signalling 1), Socs3 (Suppressor of cytokine signalling 3), Sirt1 (Sirtuin 1), Sirt5 (Sirtuin 5), Sirt6 (Sirtuin 6), α-SMA (α-smooth muscle actin), MCP1 (monocyte chemoattractant protein 1), TGFβ1 (transforming growth factor beta 1), or F4/80 antigen (macrophage biomarker) and the changes in the phosphorylations of STAT3 (Signal transducer and activator of transcription 3), c-Src, FAK(focal adhesion kinase), mTOR, S6K, ULK (UNC-51-like kinase), 4EBP1(Eukaryotic translation initiation factor 4E-binding protein 1) and Akt proteins related to obesity due to fatty liver, hepatitis, fibrosis, cancer development and metabolic disorders, depending on the expression of TM4SF5 in the cells and the tissues obtained from cells and mice, by confirming the disease characteristics of the liver tissue by comparing cell damage, cell arrangement pattern disorder, collagen I or laminin synthesis/accumulation, and AFP (Alpha-fetoprotein), FUCA (AFU, Alpha-L-fucosidase), CD34 (human hematopoietic stem cell and endothelial cell marker), HIF1α (Hypoxia-inducible factor 1-alpha), Ki-67 (Antigen KI-67), or Cyclin D1 expression/accumulation, by confirming the level of triglyceride (TG), free fatty acid (FFA), cholesterol, alanine aminotransferase (ALT), aspartate aminotransferase (AST), LDL (Low-density lipoprotein), glucose or insulin in plasma samples of animals, by measuring the increase in weight, and by measuring the increase in weight/liver weight.

### 2. Description of the Related Art

The liver has many functions such as metabolism of lipids, detoxification, bile excretion, storage of various nutrients, hematopoiesis, blood clotting and regulation of circulating blood volume. Therefore, when the liver failure occurs, various functions are degraded, and in the worst case, life is difficult to maintain.

More particularly, the functions of the liver are as follows. First, the liver has a function of managing energy metabolism, so all nutrients such as carbohydrates, fats and proteins including amino acids absorbed from food are metabolized as substances capable of producing energy in the liver and are supplied to or stored in the body. Second, about 2,000 enzymes, albumin, and coagulation factors present in the liver synthesize, store, and distribute bile acids, phospholipids and fats such as cholesterol. Third, the liver has the functions of detoxification and decomposition. The liver detoxifies drugs, alcohol, and toxic substances, so it is easy to damage liver cells during this process. Therefore, liver diseases caused by drugs, poisons, or alcohol can often occur. In addition, the liver has the function of excreting various metabolites into the duodenum, and immune function, etc., so the liver is important for maintaining life.

Liver disease can be classified into viral liver disease, alcoholic liver disease, drug toxic liver disease, fatty liver, autoimmune liver disease, metabolic liver disease, and others depending on the cause. Liver disease is the first cause of death in the world as well as in Korea, as it is found only after considerable progress because there is no initial symptom. Therefore, research on an effective diagnosis method and a treatment method for liver disease is required.

When the liver is stimulated by alcohol, viruses, or harmful environmental factors, hepatic stellate cells are activated to secrete various cytokines including TGF (transforming growth factor (3). TGFβ is a cytokine known to play an important role in the development and carcinogenesis process. The TGF receptor phosphorylates and activates intracellular Smad2/3 proteins by the activated TGFβ, binds to Smad4, which moves into the nucleus, and promotes the transcription of several related genes.

Many of the proteins whose expression is regulated by TGFβ1 are associated with the induction of fatty liver and steatohepatitis. If metabolic function is abnormally regulated through the changes in expression of the proteins whose expression is regulated by TGFβ1, the expressions of fat biosynthesis-related enzymes, signal transduction proteins or enzymes and proteins involved in the absorption and accumulation of fat are regulated to increase as nutrients such as carbohydrates, fats, or proteins (including amino acids) are ingested excessively. So, fat is accumulated in the liver epithelial cells, fatty liver (steatosis) develops, and steatohepatitis (steatohepatitis) is induced if inflammation develops further.

The fat biosynthesis-related enzymes or signal transduction proteins or factors include Srebp1, Srebp2, Fasn, Pparα, Pparγ, Leptin, Accα, Accβ, Sirt1, Sirt5, Sirt6, insulin, or glucose, and the enzymes and proteins involved in the absorption and accumulation of fat include CD36, Fabp1, Vldlr, Ldlr, ApoB100 and the like. If the fatty liver becomes severe due to the above reasons, steatohepatitis accompanied by inflammation may occur, the amount of triglyceride or triacylglycerol in plasma, free fatty acid and cholesterol (VLDL and LDL) is increased, the symptoms of obesity or abdominal obesity may be induced, and the weight can be increased.

On the other hand, TGF promotes the synthesis of collagen to induce liver fibrosis, and affects not only hepatic stellate cells but also surrounding hepatocytes, causing EMT (epithelial to mesenchymal transition). If liver fibrosis persists, cirrhosis is eventually induced, so understanding the process of liver fibrosis is necessary to treat cirrhosis.

A lot of cytokines such as TGFβ1 are secreted by inflammation. Hepatic stellate cells and other hepatocytes are activated by the secreted cytokines, and many extracellular matrixes such as collagen I, fibronectin and laminin are synthesized and accumulated outside cells. In this case, the amount of mRNA and protein of MCP1 or F4/80 antigen, the inflammation-related factor, may be increased, and damage of cells in tissue, cell arrangement pattern disorder, or synthesis accumulation of collagen I or laminin may occur.

Alcoholic liver damage is caused by alcohol itself or by the compounds produced in the metabolic process of alcohol, which leads to lipid accumulation, hepatocellular damage and fibrosis. In addition, if hepatocytes are damaged by various causes such as chronic hepatitis B, chronic hepatitis C, chronic autoimmune disease, chronic biliary tract disease, chronic heart disease, parasites and drug intoxication, various cytokines and reactive oxygen species are produced by the interaction of various cells, such as hepatocytes, Kupffer cells, sinusoidal endothelial cells and hepatic stellate cells. Due to this, the extracellular matrix (ECM) is damaged, and abnormal proliferation of ECMs such as collagen I and III is induced, thereby leading to liver fibrosis.

In general, hepatic fibrosis is reversible, unlike cirrhosis, composed of thin fibrils, and nodules are not formed therein. In addition, liver fibrosis can be restored to normal if the cause of liver damage disappears, but if recurrence of liver fibrosis is repeated, cross-linking between ECMs increases to form thin microfibers and it progresses to irreversible cirrhosis with nodules. This cirrhosis is a chronic disease that pathologically involves necrosis, inflammation and fibrosis, and ultimately progresses to liver cancer if neglected.

In general, it is known that the liver tissue of liver cancer patients has increased mRNA or protein expression of AFP (Alpha-fetoprotein), FUCA (AFU, Alpha-L-fucosidase), CD34 (human hematopoietic stem cell and endothelial cell marker), HIF1α (Hypoxia-inducible factor 1-alpha), Ki-67 (Antigen KI-67) or Cyclin D1.

Meanwhile, TM4SF5 (transmembrane 4 L6 family member 5) protein is known as a type of tetraspanin. The TM4SF5 protein is a water-insoluble protein and includes four regions that pass through the cell membrane, two ring structures outside the cell, one ring structure present in the cytoplasm, and two terminal structures. These proteins form a giant tetraspanin-web or a tetraspanin-enriched microdomain (TERM) complex in the cell membrane with cell adhesion molecules such as integrin. This complex contributes to various biological functions such as cell adhesion, proliferation and migration. TM4SF5 protein is known to be overexpressed in human liver cancer cells.

In this regard, Korean Patent No. 10-0934706 discloses a method for screening anticancer substances using the cancer cells expressing TM4SF5 protein and an anticancer composition comprising a compound that inhibits the activity of TM4SF5 protein.

The present inventors tried to develop a method to diagnose liver diseases by using the expression changes of TM4SF5 protein. In the course of our study, the present inventors confirmed in the liver tissue or hepatocytes obtained from the TM4SF5 protein over-expressing transgenic mouse or the Tm4sf5 gene knockout transgenic mouse (KO mouse) that (1) the expression changes of Srebp1 (Sterol regulatory element-binding protein 1), Srebp2 (Sterol regulatory element-binding protein 2), Fasn (Fatty acid synthase), CD36 (cluster of differentiation 36), Fabp1 (Fatty Acid-Binding Protein 1), Vldlr (very-low-density-lipoprotein receptor), Ldlr (low density lipoprotein receptor), ApoB100 (Apolipoprotein B 100), Pparα (Peroxisome proliferator-activated receptor alpha), Pparγ (Peroxisome Proliferator Activated Receptor Gamma), Leptin, Accα (acetyl-CoA carboxylase alpha), Accβ (acetyl-CoA carboxylase beta), collagen I, collagen type I alpha 1 chain, laminins, laminin α5, laminin γ2, laminin γ3, Socs1 (Suppressor of cytokine signalling 1), Socs3 (Suppressor of cytokine signalling 3), Sirt1 (Sirtuin 1), Sirt5 (Sirtuin 5), Sirt6 (Sirtuin 6), α-SMA (α-smooth muscle actin), MCP1 (monocyte chemoattractant protein 1), TGFβ1 (transforming growth factor beta 1) or F4/80 antigen (macrophage biomarker) mRNA or protein, (2) the changes in the phosphorylation of STAT3 (Signal transducer and activator of transcription 3) protein, Src (cellular sarcoma) protein, FAK (focal adhesion kinase) protein, FAK (focal adhesion kinase), mTOR, S6K, ULK, 4EBP1 and Akt protein, (3) the disease characteristics of the liver tissue by comparing cell damage, cell arrangement pattern disorder, collagen I or laminin synthesis/accumulation, and AFP (Alpha-fetoprotein), FUCA (AFU, Alpha-L-fucosidase), CD34 (human hematopoietic stem cell and endothelial cell marker), HIF1α (Hypoxia-inducible factor 1-alpha), Ki-67 (Antigen KI-67), or Cyclin D1 expression/accumulation, (4) the level of triglyceride (TG), free fatty acid (FFA), cholesterol, alanine aminotransferase (ALT), aspartate aminotransferase (AST), LDL (Low-density lipoprotein), glucose or insulin in plasma samples of animals, and (5) TM4SF5 played a positive role in developing fatty liver, steatohepatitis and liver fibrosis by measuring weight gain and weight/liver weight increase. The present inventors further confirmed, while the transgenic mouse continued to be raised, that the expression pattern of the mRNA and protein above had been changed and the phosphorylation pattern of the protein above had been changed to express the symptoms of liver fibrosis, hepatitis, liver cirrhosis or liver cancer.

In the Tm4sf5 gene knockout (KO) mouse, there were no significant changes in the expression pattern or the phosphorylation pattern of mRNAs and proteins of the confirmed factors; the degree of glucose resistance, insulin resistance or weight gain caused by the high-fat diet, high-carbohydrate diet, high-amino acid (arginine) diet, or high-sucrose diet which can cause obesity and metabolic disease was weak; and the levels of plasma triglyceride, cholesterol and AST/ALT were not much increased. Therefore, it was confirmed by the results above that the outbreak of liver diseases including fatty liver, hepatitis, liver fibrosis, liver cirrhosis and liver cancer can be induced by the expression of TM4SF5, leading to the completion of the present invention.

### PRIOR ART REFERENCE

### Patent Reference

(Patent Reference 1) Korean Patent No. 10-0934706

### SUMMARY OF THE INVENTION

It is an object of the present invention to provide a method for diagnosing liver diseases using the expression changes of TM4SF5 protein.

It is another object of the present invention to provide a method for screening a candidate substance for treating liver diseases or an anti-obesity candidate substance using the expression changes of TM4SF5 protein.

It is another object of the present invention to provide a method for preparing an animal model of portal hypertension using a TM4SF5 gene knock-out mouse and an animal model prepared by the method.

To achieve the above objects, the present invention provides a method of providing information for the diagnosis of liver diseases comprising the following steps:

1) selecting a sample, in which the expression level of TM4SF5 (transmembrane 4 L6 family member 5) protein is increased, obtained from a suspected liver disease patient compared to a sample obtained from a normal control group;

2) measuring the expression level of SREBP1 (sterol regulatory element-binding transcription factor 1) mRNA or protein, and the phosphorylation level of one or more proteins selected from the group consisting of STAT3 (signal transducer and activator of transcription 3) protein, c-Src (cellular sarcoma) protein, FAK (focal adhesion kinase) protein, mTOR, S6K, ULK, 4EBP1 and Akt proteins in the sample selected in step 1); and

3) comparing the expression level of SREBP1 mRNA or protein, and the phosphorylation level of one or more proteins selected from the group consisting of STAT3 (signal transducer and activator of transcription 3) protein, c-Src (cellular sarcoma) protein, FAK (focal adhesion kinase) protein, mTOR, S6K, ULK, 4EBP1 and Akt proteins in the sample selected in step 1) measured in step 2) with the expression level of SREBP1 mRNA or protein, and the phosphorylation level of one or more proteins selected from the group consisting of STAT3 (signal transducer and activator of transcription 3) protein, c-Src (cellular sarcoma) protein, FAK (focal adhesion kinase) protein, mTOR, S6K, ULK, 4EBP1 and Akt proteins in the normal control group sample.

The present invention also provides a method for screening a candidate substance for treating fatty liver comprising the following steps:

1) treating a test substance to the cells expressing TM4SF5 and SREBP1 proteins;

2) measuring the expression level of SREBP1 mRNA or protein, and the phosphorylation level of one or more proteins selected from the group consisting of STAT3 protein, c-Src protein, FAK protein, mTOR, S6K, ULK, 4EBP1 and Akt proteins in the cells of step 1); and

3) selecting a test substance that suppresses the expression level of SREBP1 mRNA or protein and increases the phosphorylation level of one or more proteins selected from the group consisting of STAT3 protein, c-Src protein, FAK protein, mTOR, S6K, ULK, 4EBP1 and Akt proteins in the cells of step 1), or suppresses the expression level of SREBP1 mRNA or protein and reduces the synthesis of monoacyl-, diacyl- or triacyl-glycerol in step 2) compared to the control group not treated with the test substance.

The present invention also provides a method for screening a candidate substance for treating obesity, fatty liver or liver cancer comprising the following steps:

1) treating a test substance to the cells or the animal model expressing TM4SF5 protein;

2) measuring the binding of TM4SF5 protein to any one or more selected from the group consisting of mTOR protein, SLC7A1 protein and arginine in the cells or the animal model of step 1);

3) measuring the phosphorylation level of mTOR protein, S6K protein, UNC-51-like kinase 1 (ULK1) protein or 4EBP1 protein in the cells or the animal model of step 1);

4) measuring the level of monoacyl-, diacyl- or triacyl-glycerol in the cells or the animal model of step 1);

5) measuring any one or more selected from the group consisting of weight gain, glucose resistance, insulin resistance and glycolysis reactivity in the cells or the animal model of step 1);

6) measuring the expression levels of the genes related to glycolysis in the cells or the animal model of step 1); and

7) selecting a test substance that suppresses the binding of TM4SF5 protein to any one or more selected from the group consisting of mTOR protein, SLC7A1 protein and arginine in step 2), inhibits the phosphorylation of mTOR protein, S6K protein, UNC-51-like kinase 1 protein or 4EBP1 protein in step 3), reduces the level of monoacyl-, diacyl- or triacyl-glycerol in step 4), and decreases the weight gain, glucose resistance, insulin resistance or glycolysis reactivity in step 5).

The present invention also provides a method for preparing a portal hypertension animal model comprising the step of mating a TM4SF5 knock-out (KO) mouse with a mouse having the genotype of APC^{min/+} (adenomatous polyposis coli^{min/+}).

In addition, the present invention provides a portal hypertension animal model prepared by the above method.

### Advantageous Effect

The present invention can be effectively used to diagnose obesity and liver disease or to screen a candidate substance for treating obesity or liver disease by measuring the expression changes of TM4SF5 protein by confirming that the metabolic function is reduced in the cells and transgenic mice over-expressing TM4SF5 protein; the weight is gained; the expression and accumulation of mRNAs and proteins of the factors involved in the biosynthesis of fat including TM4SF5 expression-dependent proteins such as SREBP1 protein are increased by high carbohydrate, fat and amino acid diet; the characteristics of obesity, fatty liver and hepatitis appear by reducing the phosphorylation of any one or more proteins selected from the group consisting of STAT3 protein, c-Src protein, FAK protein, mTOR protein, S6K protein, ULK protein, 4EBP1 protein and Akt protein; and the expression of SREBP1 protein is decreased, the phosphorylation of STAT3 protein is increased, and the accumulation of extracellular matrix such as collagen and laminin is increased, indicating the characteristics of liver fibrosis or cirrhosis when the transgenic mice continues to be raised.

### BRIEF DESCRIPTION OF THE DRAWINGS

FIG. 1(A) is a diagram showing the construct expressing TM4SF5 protein and FIG. 1(B) is the results of confirming the expression of TM4SF5 gene in the liver tissue of the transgenic mouse introduced with the construct above.

FIG. 2(A) is a photograph of the liver tissue of the transgenic mouse (52 weeks old) over-expressing TM4SF5 protein; FIG. 2(B) is a photograph of the results of staining the liver tissue of the mouse with H&E, Oil Red 0 or Mason's trichrome; FIG. 2(C) is a graph showing that the phosphorylation level of STAT3 was low in the liver tissue of the animal (1 year old) over-expressing TM4SF5, and the expression level of SREBP1 was high (Fatty liverhigh) or low (fatty liver^{low}), compared to the normal control group (normal); and FIG. 2(D) is a graph showing the results of confirming the levels of triglyceride, albumin, and ALT in plasma of the mouse.

FIG. 3(A) shows the results of confirming the expressions of the fatty liver-related gene in the liver tissue of the transgenic mouse (52 weeks old) over-expressing TM4SF5 protein and FIG. 3(B) shows the results of confirming the expressions of the fatty liver-related protein in the liver tissue of the transgenic mouse (52 weeks old) over-expressing TM4SF5 protein, and FIG. 3(c) is the results of immunostaining the liver tissue of the mouse.

FIG. 4(A) shows the fat accumulation in the hepatocytes isolated from the animal over-expressing TM4SF5 protein, and FIGS. 4(B) and 4(C) shows the results of confirming the expression changes of the fat-related genes, and FIG. 4(D) is the analysis information for ApoB100, Ldlr, Srebp2, Pparγ, and leptin genes that increase in the normal animal but have minimal increase in the knockout animal liver tissue when the normal or Tm4sf5^{-/+} knockout animal was fasted and then refed.

FIG. 5(A) shows the results of confirming the expression of SREBP1 protein, the phosphorylation pattern of STAT3 protein, and the expression of PPAR□□protein in the hepatocytes over-expressing TM4SF5 protein; FIG. 5(B) shows the results of confirming the interaction between the phosphorylation of STAT3 protein and the expression of SREBP1 protein by treating hepatic epithelial cells with free fatty acid, and the results of confirming the interaction of SREBP1 protein expression of oxidized STAT3 protein with hepatic epithelial cells by treating free fatty acid; and FIG. 5(C) shows the results of confirming the phosphorylation changes of STAT3 protein by increasing the expression of SREBP1 protein.

FIG. 6(A) is a set of diagrams showing the results of confirming the inhibition of the production of fat; FIG. 6(B) shows the inhibition of the expression of the fat-related genes; and FIG. 6(C) shows the phosphorylation of SREBP1 (precursor pSREBP1 and mature mSREBP1) with increased expression as adipocytes (3T3-L1) differentiate, Pparγ, and STAT3, the amount of which decreases as adipocytes differentiate, in adipocytes wherein the expression of TM4SF5 protein is suppressed.

FIG. 7(A) is a set of diagrams showing the results of confirming the expression changes of SIRT genes the liver tissue of the transgenic mouse (52 weeks old) over-expressing TM4SF5 protein; FIG. 7(B) shows the expression changes of SOCS proteins; FIG. 7(C) shows the expression changes of SOCS genes; and FIG. 7(D) shows the expression changes of SOCS3 protein after culturing the hepatic epithelial cells expressing TM4SF5 protein treated with the culture fluid of adipocyte progenitor cells.

FIG. 8(A) is a set of diagrams showing the results of confirming the expression changes of SOCS1 and SOCS3 genes; FIGS. 8(B) and 8(C) shows proteins in the hepatic epithelial cells over-expressing TM4SF5 protein or in the hepatic epithelial cells treated with free fatty acid; FIG. 8(D) shows the expression changes of SOCS1 and SOCS3 proteins in the hepatocytes over-expressing SREBP1 protein; and FIG. 8(E) shows the results confirming that the amount of SREBP1 protein decreases and the phosphorylation of STAT3 protein increases when the expression of SOCS3 protein is inhibited in the primary hepatic epithelial cells isolated from the transgenic mouse (52 weeks old) over-expressing TM4SF5 protein.

FIG. 9(A) is a set of diagrams showing the results of confirming that the ratio of liver weight/weight is reduced in the case of the knockout mouse in each male and FIG. 9(B) is a diagrams of female, compared to that of the normal animal, by measuring the liver weight and weight of the normal animal (WT), Tm4sf5 gene KO mouse (Exon 1-KO, a KO mouse prepared by the method of Example 7 or Exon 3-KO, a mouse prepared by Macrogen), or heterozygote Exon 1-KO mouse after normal diet for 3 months or 6 months.

FIG. 10(A) is a set of diagrams showing the results of confirming the weekly changes in the weight of the mice when the normal animal (WT) and Tm4sf5 gene knockout (Tm4sf5^{-/-} KO) mouse were fed freely a normal diet (Chow) or a high-fat diet (HFD) generating 60 kCal/kg of calories for 10 weeks; FIG. 10(B) shows the total weight changes of the mice after 10 weeks; and FIG. 10(C) shows the cholesterol and FIG. 10(D) shows free fatty acid (FFA) in the liver tissue of each animal.

FIG. 11(A) is a set of diagrams showing the results of confirming the expression levels of mRNAs of Tm4sf5, FIG. 11(B) is a set of diagrams showing the results of confirming the expression levels of mRNAs of Srebp1, Srebp2, LdlR, and ApoB100 when the normal animal (WT) and heterozygote Tm4sf5 gene knockout (Tm4sf5^{-/+} KO) mouse were fed freely a normal diet (Chow) or a high-fat diet (HFD) generating 60 kCal/kg of calories for 10 weeks; and FIG. 11(C) shows the amount of cholesterol and free fatty acid in plasma.

FIG. 12(A) is a set of diagrams showing the results of confirming the expression changes of SOCS1 and SOCS3 genes in TM4SF5 gene knockout (KO) mouse and FIG. 12(B) shows proteins; FIG. 12(C) shows the accumulation of fat in the mouse fed a high fat diet (HFD); and FIG. 12(D) shows the expression changes of mRNAs and proteins of the fat-related genes.

FIG. 13(A) is a set of diagrams showing the results of confirming the expression changes of TM4SF5 and APC genes in the offspring obtained by crossing TM4SF5 gene KO mouse and APC^{min/+} mouse; FIG. 13(B) shows the dissection results of the offspring; FIG. 13(C) shows the expression changes of β-catenin and HIF1α proteins in the liver tissue of the offspring; FIG. 13(D) shows the expression changes of collagen in the liver tissue of the offspring; and FIG. 13(E) shows the fat-related signal transduction mechanism in the liver tissue of the offspring.

FIG. 14(A) is a set of diagrams showing the results of confirming the binding of TM4SF5 protein to mTOR in the cell line over-expressing TM4SF5 protein, FIG. 14(B) is a set of diagrams about SLC7A1, and FIG. 14(C) is a set of diagrams about SLC38A9; and that the phosphorylation of S6K, UNC-51-like kinase 1 (ULK1) or 4EBP1 is increased compared to the cells in which the expression of TM4SF5 protein is suppressed when amino acids are removed and re-provided outside the cells expressing TM4SF5 protein (FIG. 14D and FIG. 14E).

FIG. 15(A) is a set of diagrams showing the results of confirming the expression changes of arginase 1, Tm4sf5, and Tm4sf4 genes in the liver tissue of TM4SF5 gene KO (Tm4sf5^{-/+}-KO) mouse; FIG. 15(B) shows that the TM4SF5 and Castor1 proteins bind more strongly to L-arginine than the control protein MetaP2; FIG. 15(C) shows that the TM4SF5 protein binds more strongly to arginine than the other similar protein TM4SF1 or TM4SF4; the concentration-dependent binding of the TM4SF5 protein in cell extract or TM4SF5 in TM4SF5-LEL domain (long extracellular loop) cell membrane extract or the TM4SF5 recombinant protein with L-arginine, and the IC₅₀ concentration indicating the binding degree (FIG. 15D and FIG. 15E); the binding between the full-length (FL), short extracellular loop (SEL), or LEL domains among TM4SF5 proteins and L-arginine (FIG. 15F); and that the TM4SF5 mutant protein having mutations in many amino acids of the LEL domain of TM4SF5 and L-arginine cannot bind (FIG. 15G).

FIG. 16(A) is a set of diagrams showing the results of confirming the weekly changes in body weight of the mice when the normal animal (WT) and Tm4sf5 gene knockout (Tm4sf5^{-/-} KO) mouse were fed freely a normal diet (Chow) or a 70% kCal high-carbohydrate diet (HCD) that gets 70% of calories from carbohydrates for 10 weeks; FIG. 16(A) shows the total weight changes of the mice after 10 weeks; FIG. 16(c) shows the levels of glucose resistance and FIG. 16(D) shows the levels of insulin resistance of each animal; and FIG. 16(E) shows the levels of AST (aspartate aminotransferase), ALT (alanine aminotransferase), and cholesterol in plasma.

FIG. 17(A) is a set of diagrams showing the results of confirming the weight change of the TM4SF5 gene KO mouse fed a high arginine (HR) diet; FIG. 17(B) shows the weight gain of the mouse compared to the starting point of the high arginine diet; FIG. 17(C) shows the accumulation of fat in the liver tissue of the mouse.

FIG. 18(A) is a set of diagrams showing the results of confirming the phosphorylation of S6K protein in the cell line expressing TM4SF5 protein; FIG. 18(B) shows the changes of glucose reactivity by the suppression of TM4SF5 protein; and FIG. 18(C) shows the expression changes of the gene involved in glycolysis by the suppression of TM4SF5 protein.

FIG. 19(A) is a set of diagrams showing the results of confirming the weekly changes in the weight of the TM4SF5 gene KO mouse fed a high-sucrose diet (high-sucrose AIN-93G diet; It has a sucrose content of 10%, which is 3 times higher than that of a normal diet with a sucrose content of 3.15%.) for 3 or 10 weeks; FIG. 19(B) is a set of diagrams showing the results of the glucose resistance and insulin resistance; FIG. 19(C) shows the levels of AST, ALT, total cholesterol (TCHO), and triacyl-glycerol (TG) in plasma; FIG. 19(D) shows the accumulation of lipid droplets in the liver tissue by H&E staining; and FIG. 19(E) shows the levels of monoacyl-, diacyl- and triacyl-glycerol.

FIG. 20(A) is a set of diagrams showing the results of confirming the phenotype of the liver tissue of the transgenic mouse (78 weeks old) over-expressing TM4SF5 protein; FIG. 20(B) is the results of statistically confirming the phenotypes of extramedullary hematopoiesis and steatohepatitis liver fibrosis; and FIG. 20(C) is the results of confirming the expression changes of the fat-related proteins in the liver tissue.

FIG. 21(A) is a set of diagrams showing the results of confirming the phosphorylation changes of SOCS protein, ECM and STAT3 in the liver tissue of the transgenic mouse (78 weeks old) over-expressing TM4SF5 protein (A); and the expression changes of the fat metabolism related genes (FIG. 21B and FIG. 21C).

FIG. 22(A) is a set of diagrams showing the results of observing the collagen accumulation in the liver tissue of the animal model induced with liver disease by the treatment of carbon tetrachloride (CCl₄) for 4 or 16 weeks; FIG. 22(B) shows the liver tissue of the TM4SF5 gene (Tm4sf5^{-/-}-KO) KO mouse model induced with liver disease by a drug; and FIG. 22(C) shows the collagen accumulation by staining.

FIG. 23(A) is a set of diagrams showing the results of confirming the expression changes of proteins related to fibrosis in the liver tissue of the animal model induced with liver disease by carbon tetrachloride (CCl₄) and FIG. 23(B) is a set of diagrams showing the results of confirming the expression changes of genes related to fibrosis in the liver tissue of the animal model induced with liver disease by carbon tetrachloride (CCl₄).

FIG. 24 is a diagram showing the results of confirming the expression changes of the fibrosis related proteins in the liver tissue of the animal model induced with liver disease by carbon tetrachloride (CCl₄) by immunostaining.

FIG. 25 is a set of diagrams showing the expression changes of collagen and laminin, and the phosphorylation changes of STAT3, STAT5 and FAK proteins by inhibiting the expression of TM4SF5 (FIG. 25A) and STAT3 (FIG. 25B) proteins using the primary hepatic epithelial cells isolated from the liver tissue of the animal model induced with liver disease by carbon tetrachloride (CCl₄).

FIG. 26(A) is a set of diagrams showing the results of confirming the expression changes of collagen, laminin and laminin γ2 proteins, and the phosphorylation changes of STAT3, FAK and c-Src proteins by IL-6; FIG. 26(B) shows the protein expression changes caused by laminin; FIG. 26(C) shows the expression changes of laminin protein, and the phosphorylation changes of STAT3 and c-Src by treating a c-Src protein activity inhibitor (PP2); and FIG. 26(D) shows the phosphorylation changes of STAT3 protein, and the expression changes of collagen and laminin proteins by suppressing the expression of TM4SF5 protein.

FIG. 27(A) is a set of diagrams showing the schematic diagram of a construct prepared to confirm whether the phosphorylation of STAT3 protein regulates the expression thereof through a promoter of laminin; and FIG. 27(B) and FIG. 27(C) show the results of confirming whether the promoter of laminin γ2 (Lamc2, FIG. 27B) or collagen 1 α1 (Col1a1, FIG. 27C) is regulated by STAT3 protein in hepatic epithelial cells (AML12) or hepatic stellate cells (LX2 cells).

FIG. 28(A) is a set of diagrams showing the results of confirming the change in the co-expression of TM4SF5 protein and laminin protein by TM4SF5 in the animal model induced with liver disease by treating carbon tetrachloride (CCl₄) for 4 or 16 weeks; the expression changes of albumin, α-SMA and collagen in the liver tissue of the animal model (FIG. 28B and FIG. 28C); and the expression changes of collagen, laminin and laminin γ2 and the phosphorylation of STAT3 in the HepG2 cells in which the expression of TM4SF5 protein is suppressed (FIG. 28D and FIG. 28E).

FIG. 29(A) is a set of diagrams showing the results of observing the liver tissue in the animal model induced with liver disease by treating carbon tetrachloride (CCl₄) after suppressing the expression of laminin or collagen; FIG. 29(B) shows the results of confirming the mRNA expression changes of TM4SF5, collagen, laminin, α-SMA and TGF proteins; and FIG. 29(C) shows the results of confirming the expression changes of TTM4SF5, collagen, laminin and laminin γ2 and the phosphorylation changes of STAT3.

FIG. 30(A) is a set of diagrams showing the results of confirming the nodules considered as the cancer tissue by observing the liver tissue of the mouse over-expressing TM4SF5 protein; the expression changes of the liver cancer markers (FIG. 30B and FIG. 30E); the expression changes of the inflammation-related genes (FIG. 30C); the expression changes of CD34, Ki67, Cyclin D1 and HIF1-α (FIG. 30D); the expression of laminin and the phosphorylation of STAT3 (FIG. 30E); and the levels of AST, ALT, albumin, LDL (low-density lipoprotein) and triglyceride in plasma (FIG. 30F).

FIG. 31(A) is a set of diagrams showing the results of observing the liver tissue in the animal model induced with liver cancer by treating diethylnitrosamine (DEN); FIG. 31(B) shows the expression changes of TM4SF5 and laminin and the phosphorylation of STAT3; and FIG. 31(C) shows the expression changes of TM4SF5, phosphorylated STAT3, laminin, laminin γ2 and collagen I by histostaining.

FIG. 32 is a diagram showing the results of confirming the expression changes of phosphorylated STAT3, laminin and collagen I in the liver cancer tissue (HCC-tumor) and the tumor-near tissue obtained from liver cancer patients.

### DESCRIPTION OF THE PREFERRED EMBODIMENTS

Hereinafter, the present invention is described in detail.

The present invention provides a method of providing information for the diagnosis of liver diseases comprising the following steps:

1) selecting a sample, in which the expression level of TM4SF5 (transmembrane 4 L6 family member 5) protein is increased, obtained from a suspected liver disease patient compared to a sample obtained from a normal control group;

2) measuring the expression level of SREBP1 (sterol regulatory element-binding transcription factor 1) mRNA or protein, and the phosphorylation level of one or more proteins selected from the group consisting of STAT3 (signal transducer and activator of transcription 3) protein, c-Src (cellular sarcoma) protein, FAK (focal adhesion kinase) protein, mTOR, S6K, ULK, 4EBP1 and Akt proteins in the sample selected in step 1); and

3) comparing the expression level of SREBP1 mRNA or protein, and the phosphorylation level of one or more proteins selected from the group consisting of STAT3 (signal transducer and activator of transcription 3) protein, c-Src (cellular sarcoma) protein, FAK (focal adhesion kinase) protein, mTOR, S6K, ULK, 4EBP1 and Akt proteins in the sample selected in step 1) measured in step 2) with the expression level of SREBP1 mRNA or protein, and the phosphorylation level of one or more proteins selected from the group consisting of STAT3 (signal transducer and activator of transcription 3) protein, c-Src (cellular sarcoma) protein, FAK (focal adhesion kinase) protein, mTOR, S6K, ULK, 4EBP1 and Akt proteins in the normal control group sample.

The term “TM4SF5 (transmembrane 4 L6 family member 5) protein” used in this specification is a protein included in tetraspanine, tetraspan or TM4SF (transmembrane 4 super family), the membrane receptor group that cross the cell membrane 4 times, and has a structure similar to each other that passes through the cell membrane four times. The TM4SF5 protein shares a structure including four hydrophobic sites that are biochemically estimated to be transmembrane domains.

The term “SREBP1 (sterol regulatory element-binding transcription factor 1) protein” used in this specification is a transcription factor that binds to the promoter of a gene and regulates the transcription thereof, and means a factor that regulates the expression of a gene involved in sterol biosynthesis. The SREBP1 protein is regulated by insulin and regulates the expression of a gene involved in glucose metabolism or fatty acid and fat production.

The term “STAT3 (signal transducer and activator of transcription 3) protein” used in this specification is a transcription factor belonging to the STAT protein family, and means a factor that transmits a signal to a lower level by being phosphorylated by cytokines and growth factors. The STAT3 protein is activated by phosphorylation at the 705^{th} tyrosine residue by interferon, EGF (epidermal growth factor), IL-5 and IL-6, etc.

The terms “Srebp1 (Sterol regulatory element-binding protein 1), Srebp2 (Sterol regulatory element-binding protein 2), Fasn (Fatty acid synthase), CD36 (cluster of differentiation 36), Fabp1 (Fatty Acid-Binding Protein 1), Vldlr (very-low-density-lipoprotein receptor), Ldlr (low density lipoprotein receptor), ApoB100 (Apolipoprotein B 100), Pparα (Peroxisome proliferator-activated receptor alpha), Pparγ (Peroxisome Proliferator Activated Receptor Gamma), Leptin, Accα (acetyl-CoA carboxylase alpha) and Accβ (acetyl-CoA carboxylase beta)” used in this specification are enzymes or proteins involved in the accumulation or synthesis of fatty acids.

The terms “collagen I, collagen type I alpha 1 chain, laminin, laminin α5, laminin γ2 and laminin γ3” used in this specification are proteins corresponding to the extracellular matrix type. The terms “Socs1 (Suppressor of cytokine signalling 1), Socs3 (Suppressor of cytokine signalling 3), STAT3 (Signal transducer and activator of transcription 3), c-Src and FAK (focal adhesion kinase)” are proteins or signaling proteins involved in fibrosis.

The terms “Sirt1 (Sirtuin 1), Sirt5 (Sirtuin 5), Sirt6 (Sirtuin 6), α-SMA (α-smooth muscle actin), MCP1 (monocyte chemoattractant protein 1), TGFβ1 (transforming growth factor beta 1) and F4/80 antigen (macrophage biomarker)” used in this specification are factors involved in inflammation of the liver tissue.

The terms “AFP (Alpha-fetoprotein), FUCA (AFU, Alpha-L-fucosidase), CD34 (human hematopoietic stem cell and endothelial cell marker), HIF1α (Hypoxia-inducible factor 1-alpha), Ki-67 (Antigen KI-67) and Cyclin D1” used in this specification are cancer cell markers or related proteins. The terms “mTOR, S6K, ULK1, 4EBP1 and Akt” are signaling proteins involved in arginine metabolism in liver cancer cells.

The terms “triglyceride (TG), free fatty acid (FFA), cholesterol, alanine aminotransferase (ALT), aspartate aminotransferase (AST), LDL (Low-density lipoprotein), glucose and insulin” used in this specification are factors related to liver tissue damage and fatty liver, hepatitis (or steatohepatitis) and liver fibrosis, and the levels can be confirmed in the animal plasma sample.

The terms “high fat diet, high carbohydrate diet, high amino acid (arginine) diet and high sucrose diet” used in this specification are diets related to obesity and metabolic disorders. It is possible to determine whether or not to induce liver disease including fatty liver, hepatitis, fibrosis and liver cancer by measuring the level of glucose resistance, insulin resistance, triglyceride, cholesterol or AST/ALT in plasma or by checking the degree of weight gain. In particular, since sucrose is decomposed into fructose and glucose in the body and used for cells, high concentration sucrose intake may have the effect of ingesting high concentration of fructose, which is included in carbonated beverages (sweetened beverages), juices, breakfast cereals, etc. for sweetness, causing metabolic diseases such as diabetes and obesity (Journal of Korean Oriental Association for Study of Obesity 2005:5(1): 121-131].

The term “liver disease” used in this specification may include obesity, metabolic disorders, glucose resistance, insulin resistance, weight gain, fatty liver, liver fibrosis, hepatitis, liver cirrhosis or liver cancer.

The TM4SF5, SREBP1, Srebp2, Fasn, CD36, Fabp1, ApoB100, Pparα, Pparγ, Leptin, Accα, Accβ STAT3, collagen type I alpha 1 chain, laminin and laminin γ2 used in the method of providing information of the present invention can be polypeptides composed of any amino acid sequence known in the art. The polypeptides can include variants or fragments of amino acids having different sequences by deletion, insertion, substitution of amino acid residues, or a combination thereof within a range that does not affect the function of the protein. The amino acid substitution in proteins or peptides that does not change the activity of the molecule as a whole is known in the art. In some cases, the polypeptide can be modified by phosphorylation, sulfation, acrylication, saccharification, methylation, farnesylation, etc.

In an embodiment of the present invention, the TM4SF5 protein can be a polypeptide composed of the amino acid sequence represented by SEQ. ID. NO: 1. The triglyceride (TG), Vldlr, Ldlr, and free fatty acid (FFA) are components of fatty acids and fats known in the art.

The method of providing information of the present invention may provide information for the diagnosis of liver disease by identifying the characteristics of TM4SF5-dependent factors, cells, tissues, or individuals, including the expression changes of SREBP1 protein and the phosphorylation level changes of STAT3 protein. The liver disease can be fatty liver, liver fibrosis, hepatitis, liver cirrhosis, or liver cancer.

The term “TM4SF5-dependent factors” used in this specification refer to factors that increase mRNA or protein in the tissues or cells by the expression of TM4SF5 protein (increase of TM4SF5 protein). In the case of fatty liver, the examples of such factors are SREBP1, SREBP2, Fasn, CD36, Fabp1, Vldlr, Ldlr, ApoB100, Pparα, Pparγ, Leptin, Accα, and Accβ. In the case of hepatitis, the examples of such factors are MCP1, TGFβ1, and F4/80 antigen. In the case of liver fibrosis, the examples of such factors are collagen I, collagen type I alpha 1 chain, laminins, laminin α5, laminin γ2, and laminin γ3. In the case of liver cancer, the examples of such factors are AFP, FUCA (AFU), CD34, HIF1a, Ki-67, and Cyclin D1.

In addition, the TM4SF5-dependent factors can include signaling proteins that increase phosphorylation in the tissues or cells according to the expression of TM4SF5 protein (increase of TM4SF5 protein), including STAT3, c-Src, FAK, mTOR, S6K, ULK1, 4EBP1, or Akt protein.

In addition, the TM4SF5-dependent factors may include factors that increase in plasma as fatty liver and hepatitis (or steatohepatitis) develop according to the expression of TM4SF5 protein (increase of TM4SF5 protein), including triglyceride (TG), free fatty acid (FFA), cholesterol, alanine aminotransferase (ALT), aspartate aminotransferase (AST), LDL (low-density lipoprotein), glucose, or insulin.

The features that occur in the TM4SF5-dependent cells, tissues, or individuals include hepatocyte damage, cell arrangement pattern disorder, or increased collagen I or laminin synthesis accumulation as liver fibrosis develops according to the expression of TM4SF5 protein (increase of TM4SF5 protein).

In animal subjects, the expression of TM4SF5 protein (increase of TM4SF5 protein) can increase body weight; body weight/liver weight; weight gain according to high carbohydrate diet, high sucrose diet, high fat diet, low fat/high carbohydrate diet and high arginine diet; insulin resistance; glucose resistance; fatty liver and steatohepatitis; synthesis of extracellular matrix such as collagen and laminin; and accumulation of liver tissue.

In the method of providing information according to the present invention, when the level of the SREBP1, SREBP2, Fasn, CD36, Fabp1, Vldlr, Ldlr, ApoB100, Pparα, Pparγ, Leptin, Accα, or Accβ protein is increased, and the phosphorylation level of any one or more proteins selected from the group consisting of the STAT3 protein, c-Src protein, FAK protein, mTOR protein, S6K protein, ULK protein, 4EBP1 protein and Akt protein is decreased compared to the normal control group, it can be determined as fatty liver.

When the expression level of mRNA or protein of the SREBP1 is increased, and the level of monoacyl-, diacyl-, or triacyl-glycerol is reduced compared to the normal control group, it can be determined as fatty liver.

It was confirmed that the expression of TM4SF5, AFP, FUCA (AFU), CD34, HIF1a, Ki-67, and Cyclin D1 is increased in the sample of a patient with liver disease including liver cancer. The TM4SF5 protein was confirmed to bind to mTOR, SLC7A1 protein or arginine. It was also confirmed that the phosphorylation of mTOR protein, S6K protein, UNC-51-like kinase 1 (ULK1) protein or 4EBP1 is increased. The binding of the TM4SF5 protein to arginine can be mediated by the 124^{th} to 129^{th} residues from the N-terminus of the TM4SF5 protein.

In the method of providing information according to the present invention, when the expression level of the SREBP1, SREBP2, Fasn, CD36, Fabp1, Vldlr, Ldlr, ApoB100, Pparα, Pparγ, Leptin, Accα or Accβ protein is reduced, the phosphorylation level of STAT3 protein, c-Src protein, FAK protein or Akt protein is increased, and the expression of collagen I, laminin, laminin γ2 or α-SMA compared to the normal control group, it can be determined as liver fibrosis, hepatitis, liver cirrhosis or liver cancer.

In the method of providing information according to the present invention, the expression level of SREBP1 protein can be regulated by any one or more proteins selected from the group consisting of SIRT1, SIRT2, SIRT4, SIRT5, SIRT6 and SIRT7. Particularly, the increased expression of SREBP1 and SREBP2 proteins can be controlled by the decrease of the expression of SIRT1, SIRT5 and SIRT6 proteins, and the increase of the expression of SIRT2, SIRT4 and SIRT7 proteins.

The sample can be any sample as long as the expression of TM4SF5, SREBP1, SREBP2, Fasn, CD36, Fabp1, Vldlr, Ldlr, ApoB100, Pparα, Pparγ, Leptin, Accα or Accβ protein and the phosphorylation level of STAT3, c-Src, or FAK protein can be changed by liver disease.

The expression level or the phosphorylation level of the protein can be measured by any method known in the art. Particularly, the expression level of the protein can be measured by any one or more methods selected from the group consisting of Western blotting, enzyme-linked immunosorbent assay (ELISA), proteomic analysis, immunohistochemical staining, immunoprecipitation and immunofluorescence. Meanwhile, the expression level of mRNA can be measured by RT-PCR, real-time PCR or RNA-Seq.

In the method of providing information according to the present invention, the phosphorylation of STAT3 protein can be regulated by any one or more proteins selected from the group consisting of SOCS1 and SOCS3. Particularly, the decrease of the phosphorylation of STAT3 protein can be controlled by the increase of the expression of SOCS1 and SOCS3 proteins, and the increase of the phosphorylation of STAT3 protein can be controlled by the decrease of the expression of SOCS1 and SOCS3 proteins.

The method of providing information according to the present invention can further include a step of measuring the expression of any one or more mRNAs or proteins selected from the group consisting of SIRT1 (NAD-dependent deacetylase sirtuin-1), SIRT5, SIRT6, SREBP2, SREBP1c, CD36, FABP1 (fatty acid-binding protein 1), FASN (fatty Acid Synthase), LDLR (low density lipoprotein receptor), VLDLR (very Low Density Lipoprotein Receptor), PPARγ (peroxisome proliferator-activated receptors γ), TIMP1 (The tissue inhibitor of metalloproteinase-1), TGFβ1 (Transforming growth factor beta 1), TNFα (tumor necrosis factor α), vimentin, MCP1 [monocyte chemotactic protein 1 (CCL2)], laminin α2, laminin α3, laminin α5, laminin γ2, laminin γ3, SOCS1 (suppressor of cytokine signaling 1), SOCS3, ApoB100 (Apolipoprotein B), PPARα, Leptin, Acc (Acetyl-CoA carboxylase)α, Accβ, F4/80 antigen, collagen I, collagen type I alpha 1 chain, AFP (Alpha-fetoprotein), FUCA (AFU, alpha-L-fucosidase 1), CD34, HIF1α (Hypoxia-inducible factor), Ki-67 and Cyclin D1. When the expression level of mRNA or protein of SIRT1, SIRT5, SIRT6, laminin α5, laminin γ2 or laminin γ3 is decreased, the expression level of mRNA or protein of SREBP2, SREBP1c, CD36, FABP1, FASN, LDLR, VLDLR, PPARγ, TIMP1, TGFβ1, TNFα, vimentin, MCP1, SOCS1, SOCS3, ApoB100, PPARα, Leptin, Accα or Accβ is increased, the level of monoacyl-, diacyl-, and triacyl-glycerol is increased, and the phosphorylation level of any one or more proteins selected from the group consisting of STAT3 protein, c-Src protein, FAK protein, mTOR protein, S6K protein, ULK protein, 4EBP1 protein and Akt protein is decreased or not changed compared to the normal control group, it can be determined as fatty liver. On the other hand, when the expression level of mRNA or protein of SREBP2, SREBP1c, CD36, FABP1, FASN, LDLR, VLDLR or PPARγ is decreased or not changed compared to the normal control group, the expression level of mRNA or protein of SIRT1, SIRT5, SIRT6, TGFβ1, TNFα, vimentin, laminin, laminin γ2, collagen SOCS1, SOCS3, F4/80 antigen, collagen I, collagen type I alpha 1 chain, AFP (Alpha-fetoprotein), FUCA (AFU, alpha-L-fucosidase 1), CD34, HIF1α (Hypoxia-inducible factor), Ki-67 or Cyclin D1 is increased, the cytokine/chemokine factors such as MCP1, TGFβ1 and F4/80 antigen is increased, or the phosphorylation level of any one or more proteins selected from the group consisting of STAT3 protein, c-Src protein, FAK protein, mTOR protein, S6K protein, ULK protein, 4EBP1 protein and Akt protein is increased compared to the normal control group, it can be determined as liver fibrosis, hepatitis, liver cirrhosis or liver cancer.

When the expression level of mRNA or protein of SREBP2, SREBP1c, CD36, FABP1, FASN, LDLR, VLDLR or PPARγ is reduced, the expression level of mRNA or protein of SIRT1, SIRT5, SIRT6, TGFβ1, TNFα, vimentin, laminin, laminin γ2, collagen I, SOCS1, SOCS3, F4/80 antigen, collagen I, collagen type I alpha 1 chain, AFP (Alpha-fetoprotein), FUCA (AFU, alpha-L-fucosidase 1), CD34, HIF1α (Hypoxia-inducible factor), Ki-67 or Cyclin D1 is increased, the expression level of mRNA or protein of AFP, FUCA (AFU), CD34, HIF1α, Ki-67, Cyclin D1, laminin, collagen I or laminin γ2 is increased, the phosphorylation level of any one or more proteins selected from the group consisting of STAT3 protein, c-Src protein, FAK protein, mTOR protein, S6K protein, ULK protein, 4EBP1 protein and Akt protein is increased compared to the normal control group, it can be determined as liver cancer.

As the expression of the TM4SF5 protein increases, the amount of any one or more selected from the group consisting of triglyceride (TG), free fatty acid (FFA), cholesterol, alanine aminotransferase (ALT), aspartate aminotransferase (AST), LDL (Low-density lipoprotein), glucose and insulin in plasma can be increased as fatty liver and hepatitis develop. As the expression of the TM4SF5 protein increases, hepatocyte damage, cell arrangement pattern disorder or increased synthesis accumulation of collagen I or laminin may be seen in the tissue as liver fibrosis develops. The expression of TM4SF5 protein can increase body weight; body weight/liver weight; weight gain according to high carbohydrate diet, high sucrose diet, high fat diet, low fat/high carbohydrate diet and high arginine diet; insulin resistance; glucose resistance; fatty liver and steatohepatitis; or synthesis of extracellular matrix such as collagen and laminin in patients.

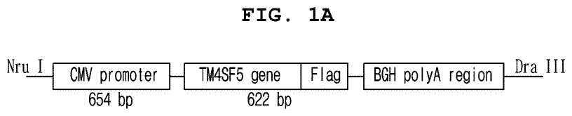

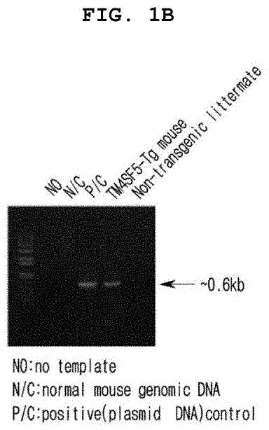

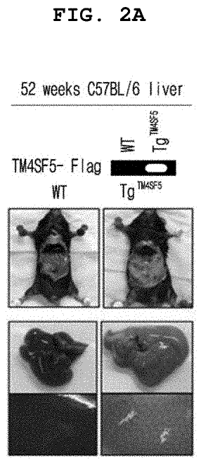

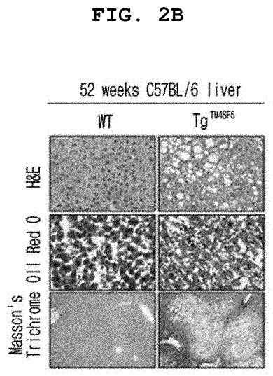

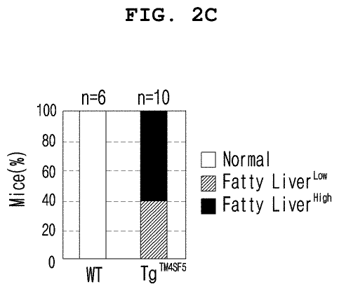

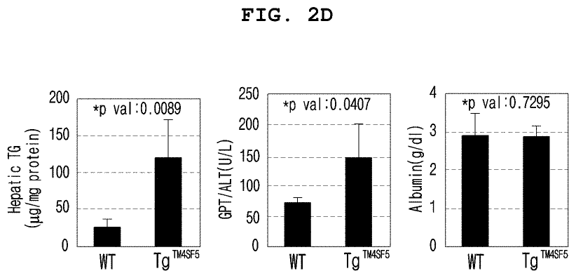

In a specific embodiment of the present invention, the present inventors prepared a mouse model (52 weeks old) transformed with a construct expressing TM4SF5 protein (see FIG. 1), and confirmed that the fat formation was promoted in the liver tissue of the mouse model (see FIG. 2).

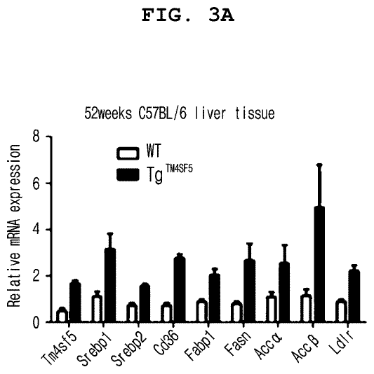

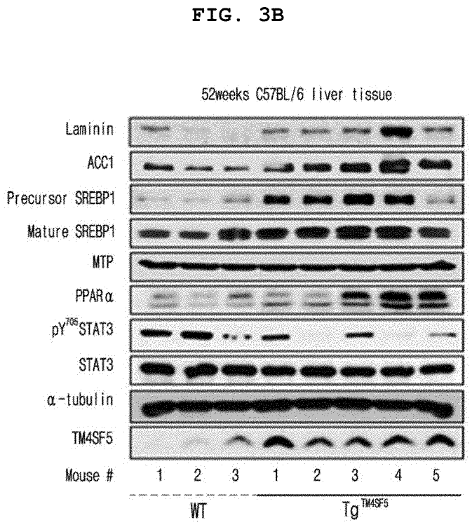

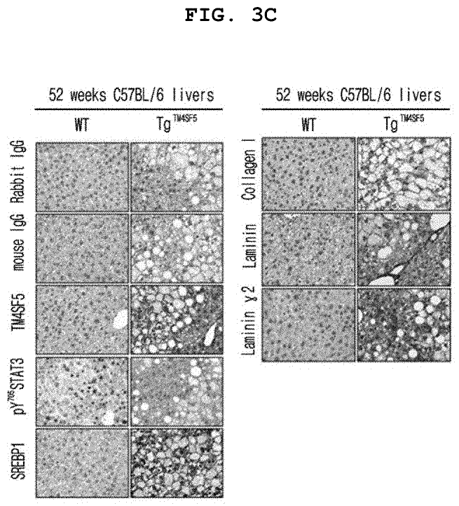

In addition, hepatocytes were obtained from the liver tissue of the prepared transgenic mouse, and the expression changes of genes and proteins related to fatty liver were confirmed. As a result, it was confirmed that the expression of mRNAs or proteins of SREBP1, SREBP2, SREBP1c, CD36, Fabp1, Fasn, Accα, Accβ, Ldlr, SOCS1 and SOCS3 was increased; the phosphorylation of STAT3 protein was reduced; and the levels of triglyceride (TG), AST and ALT in the liver tissue were increased (see FIGS. 2 and 3). When TM4SF5 gene was additionally expressed in the primary hepatic epithelial cells isolated from the transgenic mouse (52 weeks old) over-expressing TM4SF5 protein, or when the mouse was treated with free fatty acid (FFA) or IL6, fat was accumulated in the cells and the expression of mRNAs of SREBP1, SREBP2, SREBP1c, CD36, Fabp1, Fasn, Accα, Accβ, Ldlr, SOCS1 and SOCS3 was increased in the liver tissue.

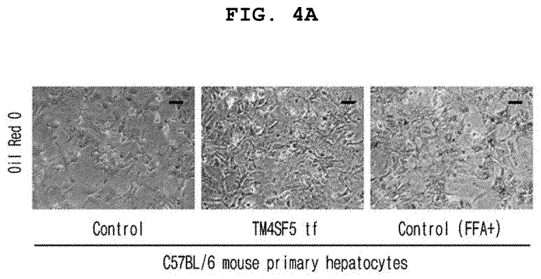

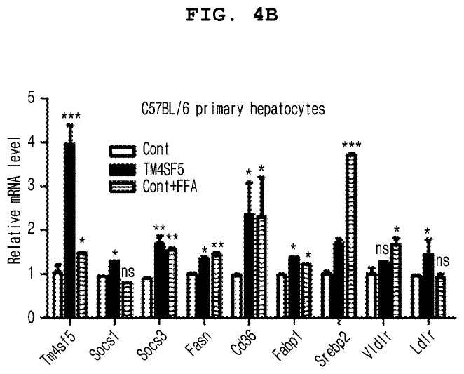

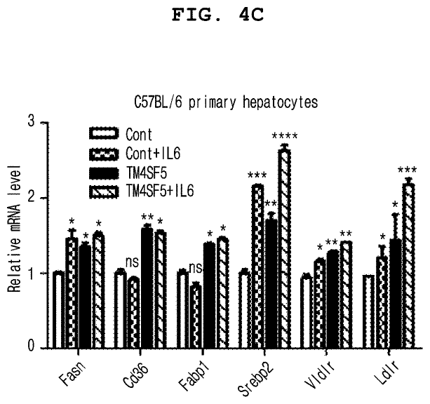

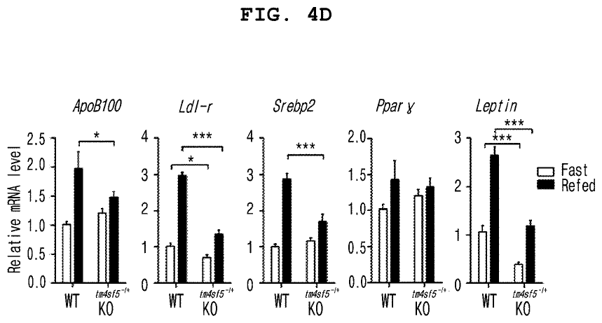

On the other hand, it was confirmed that the increase of ApoB100, LdlR, Srebp2, Pparγ and leptin was weak in the liver tissue of the animal in which Tm4sf5 gene was removed as a heterozygote compared to the normal animal not over-expressing TM4SF5 (see FIG. 4).

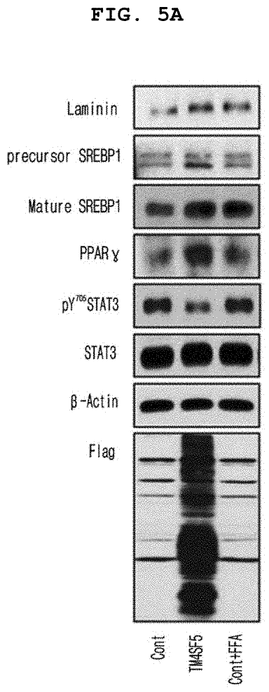

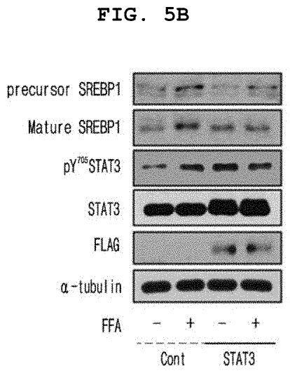

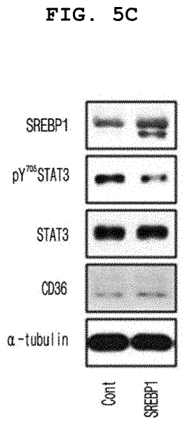

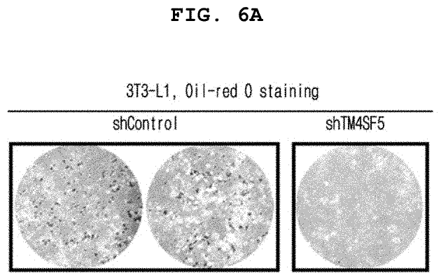

When TM4SF5 was over-expressed in a cell model or free fatty acid was treated to a TM4SF5 non-expressing cell line, it was confirmed that the increase of the expression of SREBP1 or Pparγ protein is inversely correlated with the phosphorylation of STAT3 protein (see FIG. 5).

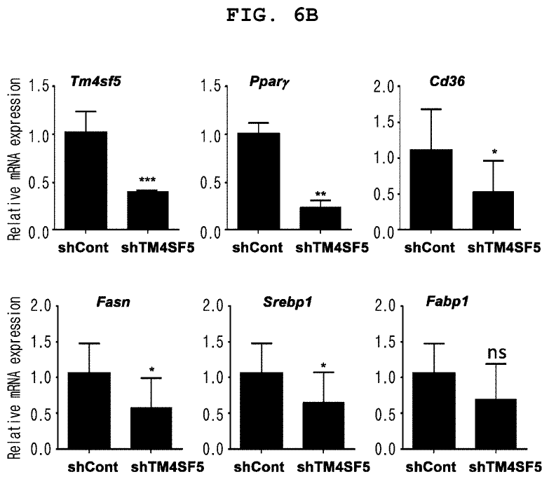

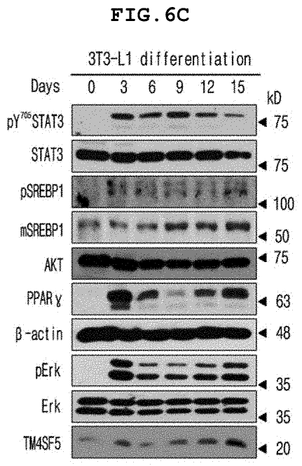

In adipocytes (3T3-L1), it was confirmed that fat was accumulated depending on the expression of TM4SF5, and the levels of mRNA and protein of Pparγ, CD36, Fasn, Srebp1 or Fabp1 were maintained (see FIG. 6).

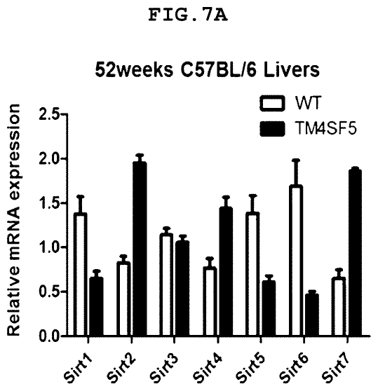

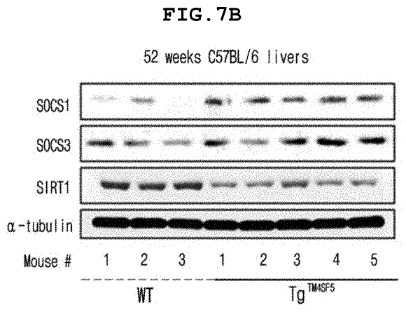

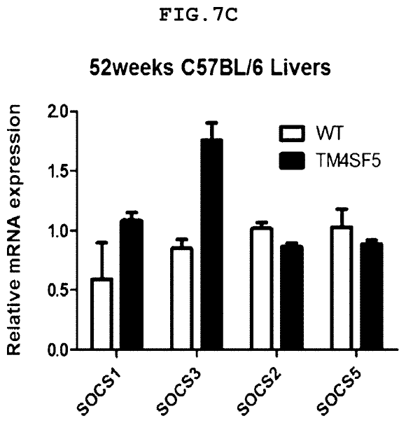

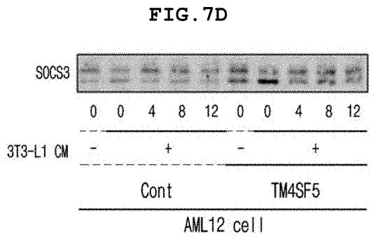

It was confirmed that the increase of the expression of mRNA or protein of SREBP1, SREBP2 or SREBP1c was increased by the decrease of the expression of SIRT1, SIRT5 or SIRT6 gene, and the increase of the expression of SIRT2, SIRT4 or SIRT7 gene. It was also confirmed that the increase of the phosphorylation of STAT3 protein was regulated by the expression of SOCS1 and SOCS3 genes and proteins (see FIG. 7).

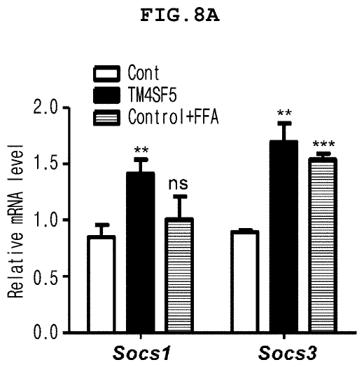

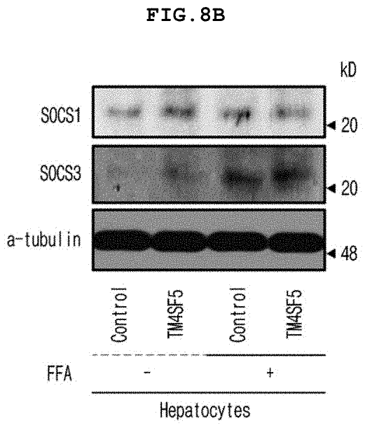

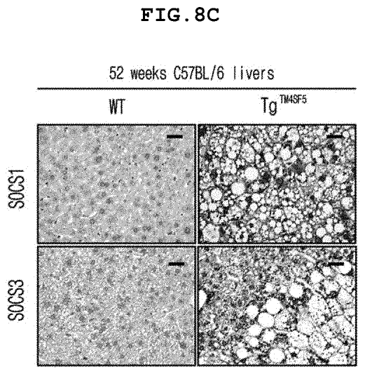

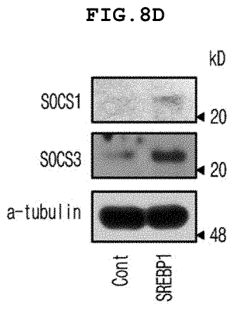

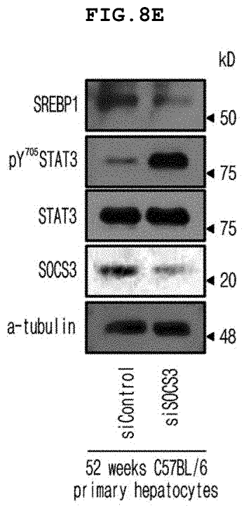

In a specific embodiment of the present invention, when TM4SF5 was expressed in the primary liver epithelial cells isolated from 52-week-old C57BL/6 normal animals or free fatty acid (FFA) was treated thereto, it was confirmed that the expression of SOCS1 and SOCS3 had positive feedback (or correlation) with the expression of TM4SF5. The expression of SREBP1 and the expression of SOCS3 were confirmed to have positive feedback, and the expressions of the proteins (Srebp1, Socs1 and Socs3) associated with the expression of TM4SF5 were negatively correlated with the phosphorylation of STAT3 protein (negative feedback) (see FIG. 8).

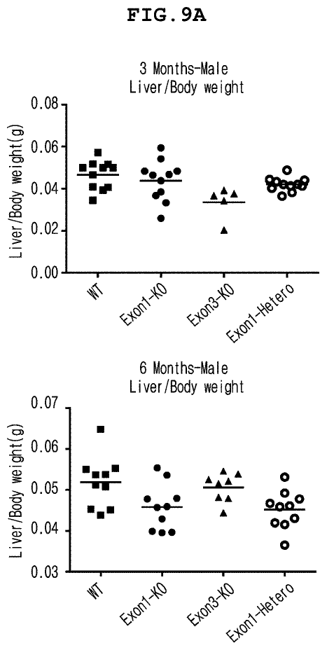

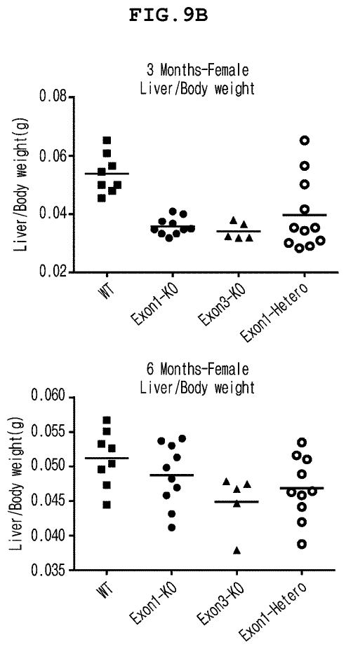

In addition, it was confirmed in the TM4SF5 gene knockout mouse (TM4SF5 gene KO mouse) that the ratio of liver weight/weight of both males and females was lower than that of the normal animals at 3 or 6 months of age (see FIG. 9).

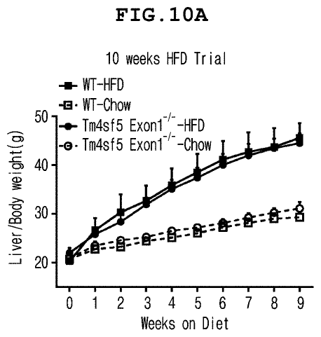

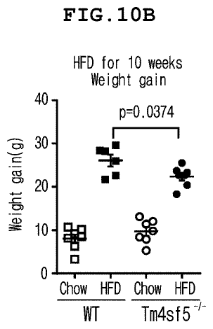

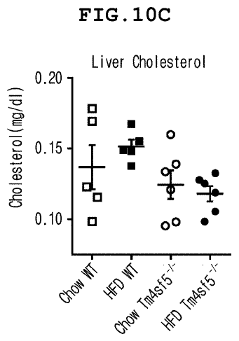

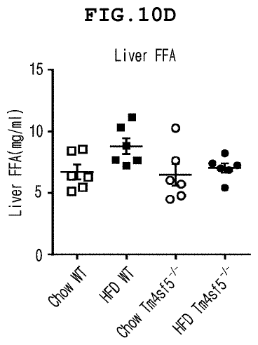

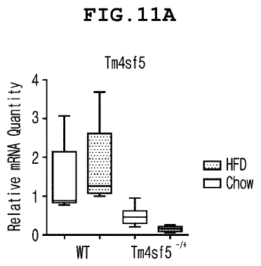

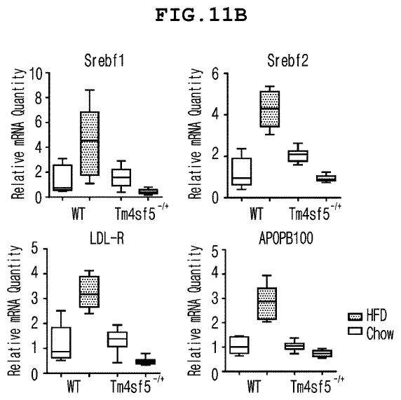

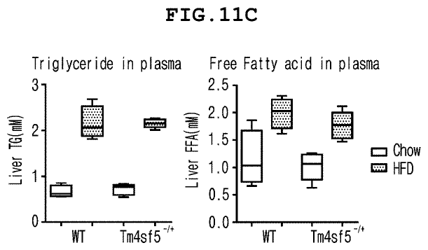

When the high fat diet was fed freely for 10 weeks, the normal animal showed a significant increase in weight compared to the normal diet, but the TM4SF5 gene knockout mouse showed a low level of weight gain and low levels of cholesterol and FFA in the liver tissue (see FIG. 10). In addition, unlike the normal animal, the expression level of Srebp1, srebp2, Ldlr or ApoB100 mRNA was not increased in the knockout mouse by the high fat diet, and the increase of triglyceride (TG) and free fatty acid (FFA) in plasma was weak (see FIG. 11).

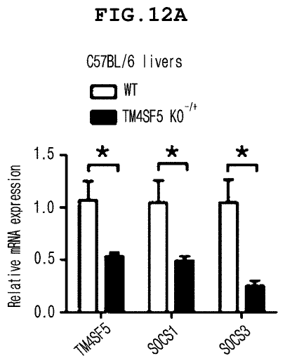

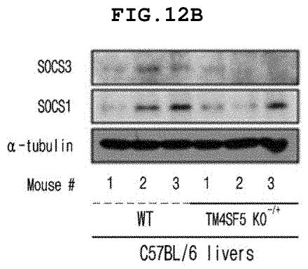

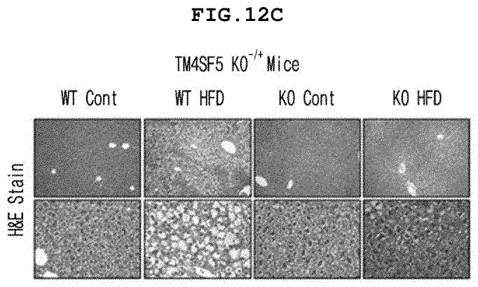

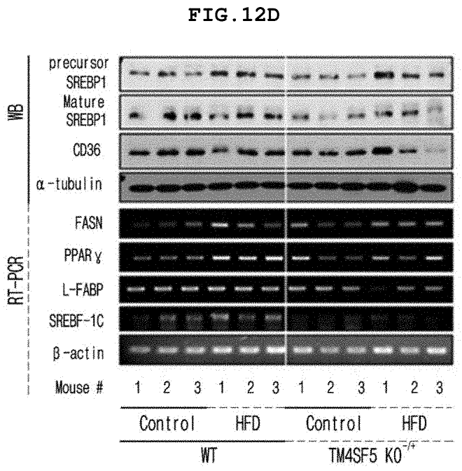

In the 52-week-old TM4SF5 knockout C57BL/6 mouse (Tm4sf5^{-/+}, the levels of Socs1 and Socs3 mRNAs and proteins were reduced compared to those in the normal mouse. When the high fat diet was fed, the normal animal showed the symptoms of steatohepatitis, but the knockout animal showed the weak symptoms, and at this time, Srebp1c mRNA and Srebp1 protein were reduced (see FIG. 12).

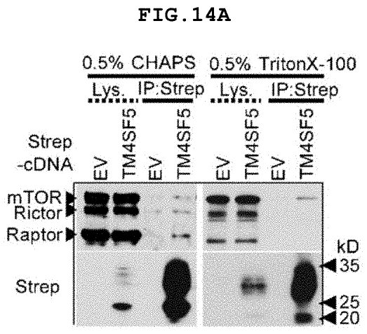

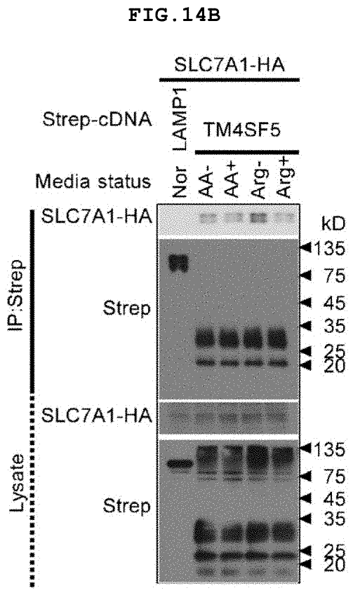

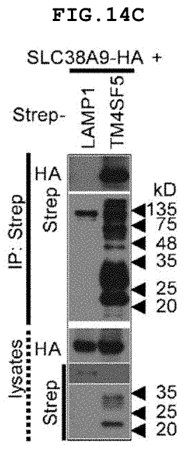

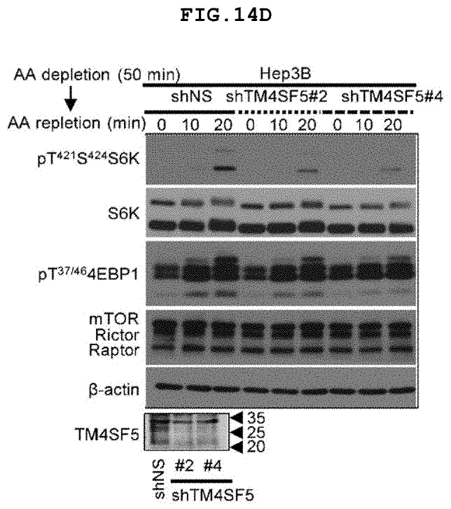

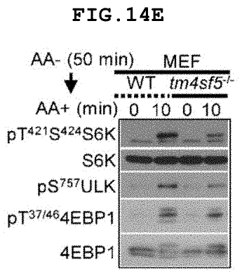

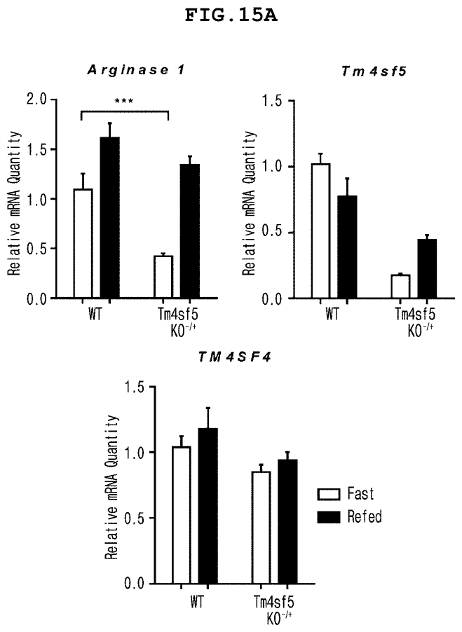

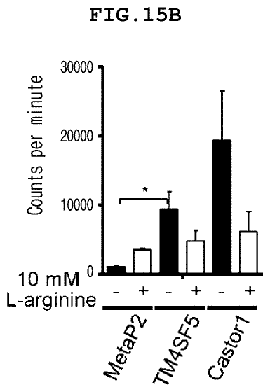

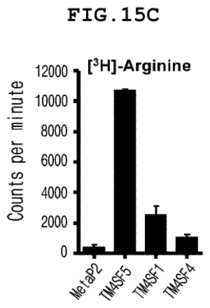

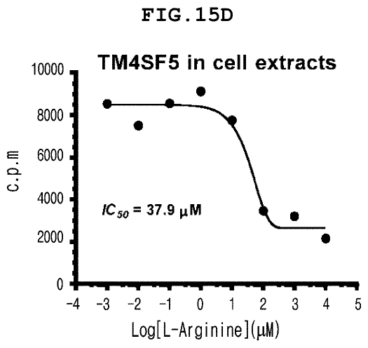

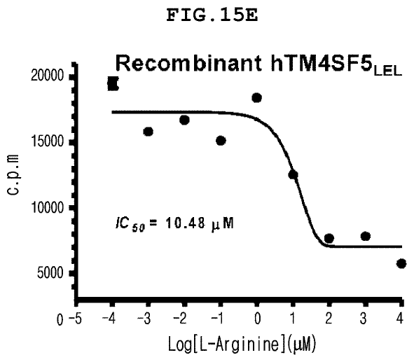

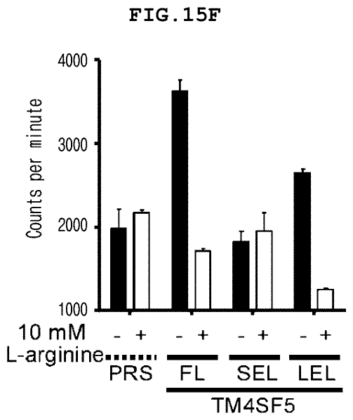

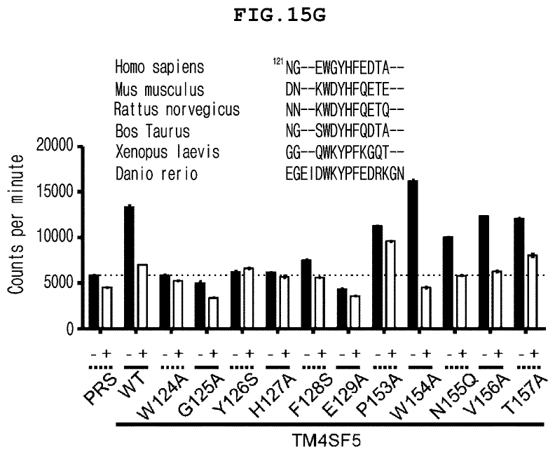

Furthermore, it was confirmed that the TM4SF5 protein was involved in arginine transport and induced S6K activity by binding to mTOR, SCL7A1 and arginine (see FIGS. 14 and 15).

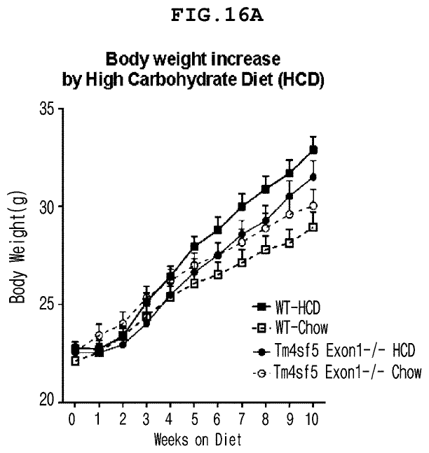

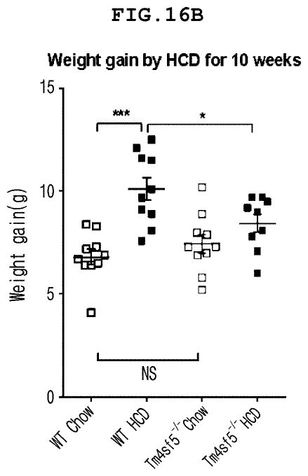

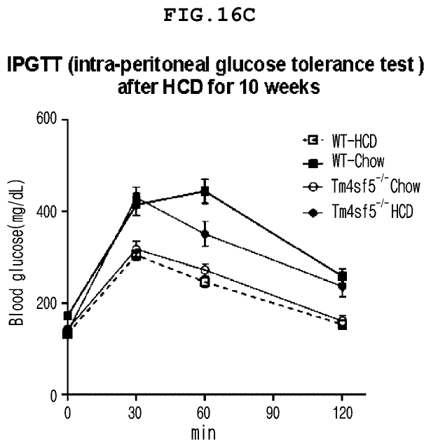

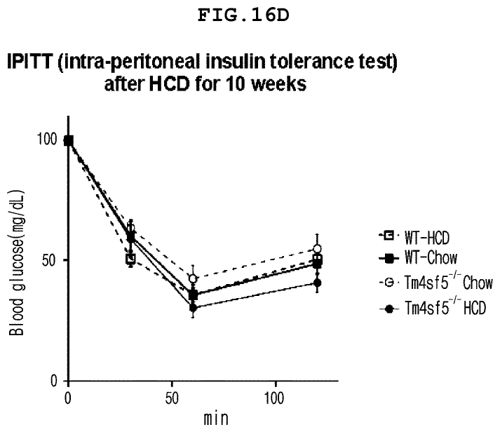

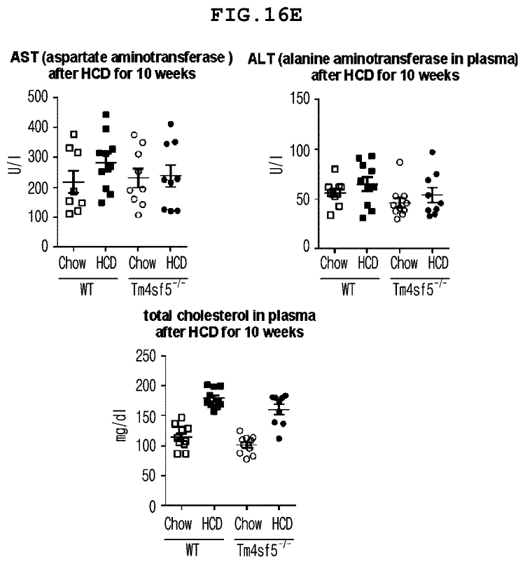

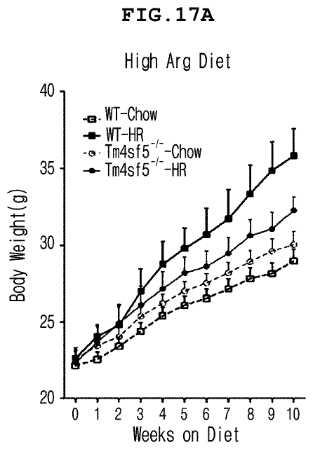

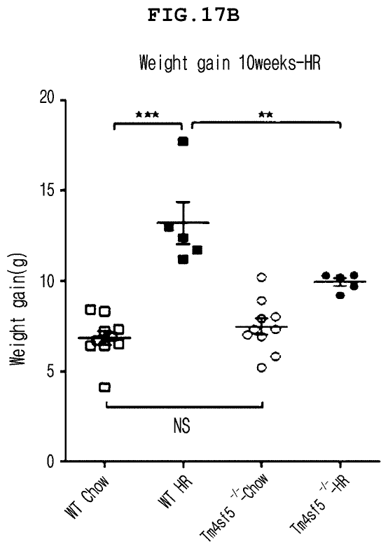

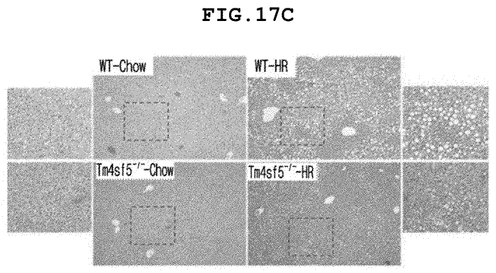

In the TM4SF5 gene KO mouse, weight gain, fat accumulation, glucose resistance, insulin resistance or liver tissue damage was suppressed even when the high carbohydrate or high arginine diet unlike in the normal mouse (see FIGS. 16 and 17).

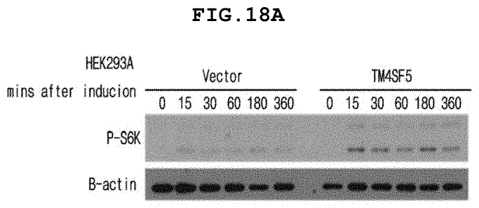

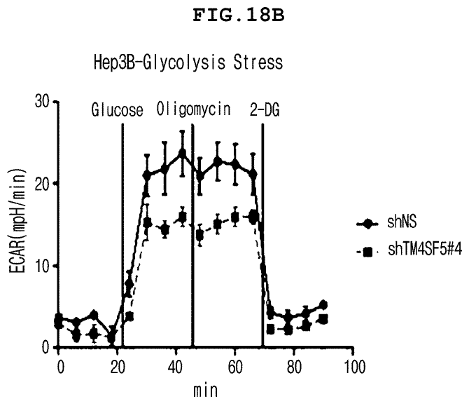

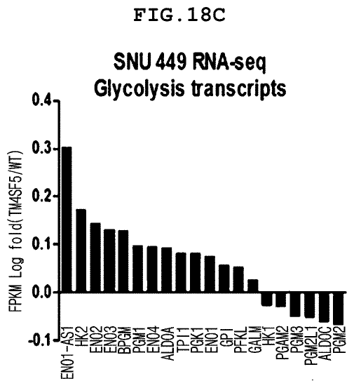

In the TM4SF5 gene KO mouse, unlike in the normal mouse, it was confirmed that the function of glycolysis for energy production was reduced by measuring the extracellular acidification rate (ECAR) by applying pharmacological stress to mitochondria. Through RNA-Seq analysis, a group of genes that depended on the expression of TM4SF5 was identified (see FIG. 18).

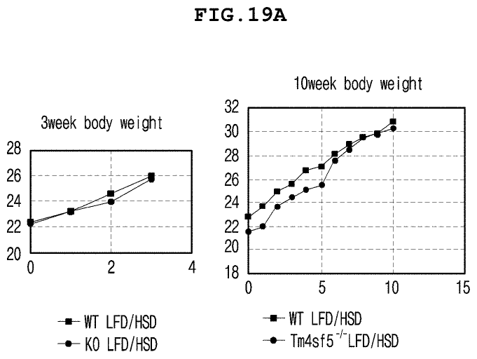

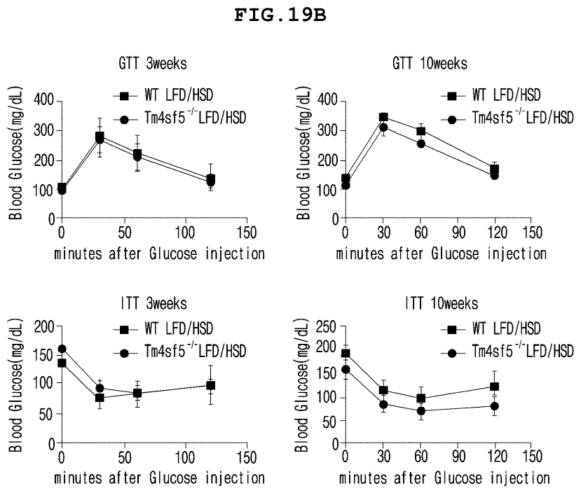

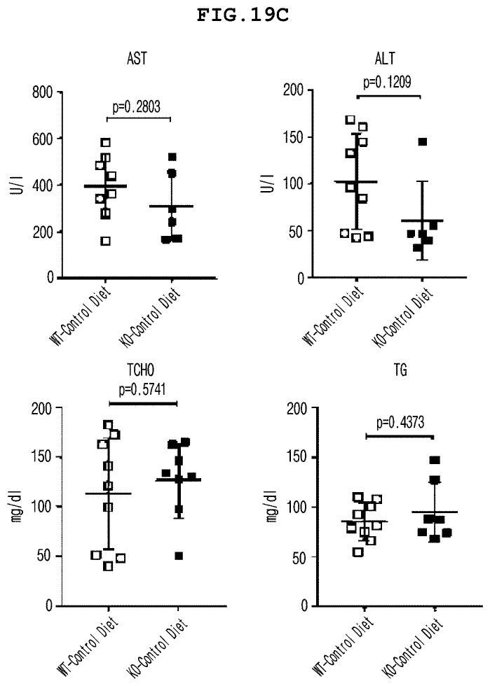

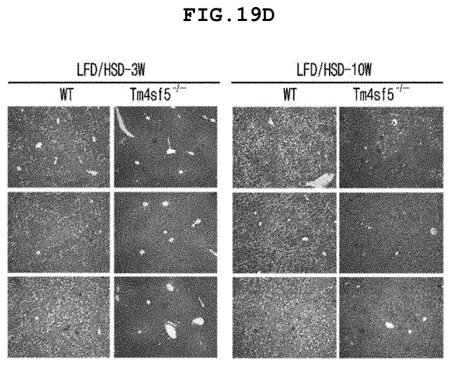

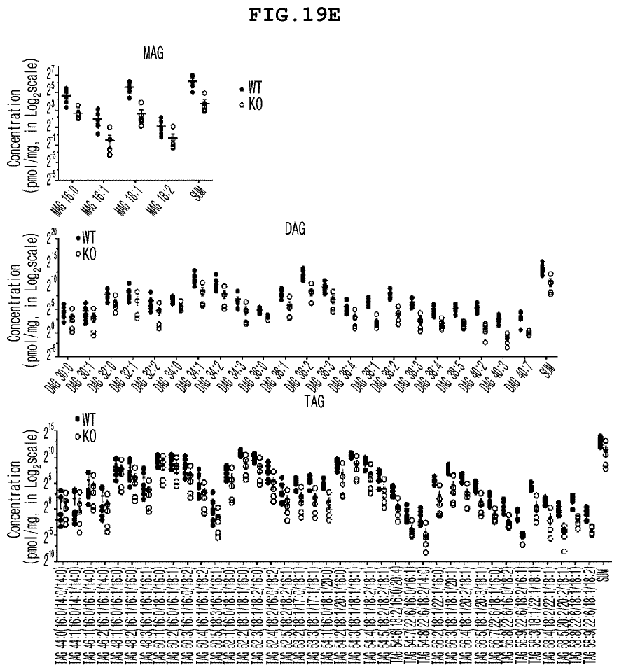

When the TM4SF5 gene KO mouse was fed a high sucrose diet, unlike the normal animal, the symptoms of fatty liver were weak, and the levels of AST, ALT and total cholesterol in plasma were low. When the lipid component was analyzed, it was confirmed that the contents of monoacyl-, diacyl- and triacyl-glycerol were low in the Tm4sf5 gene KO mouse compared to those in the normal mouse (see FIG. 19).

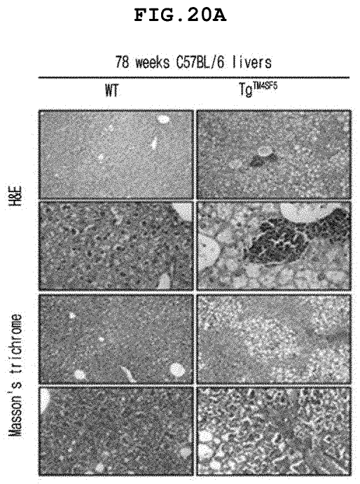

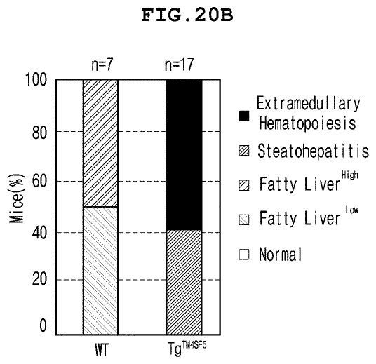

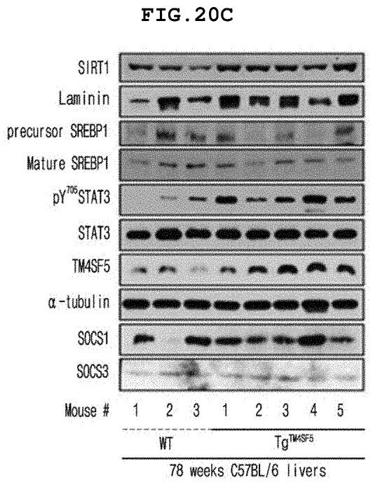

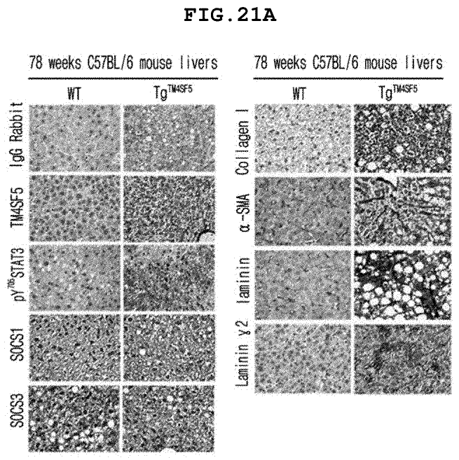

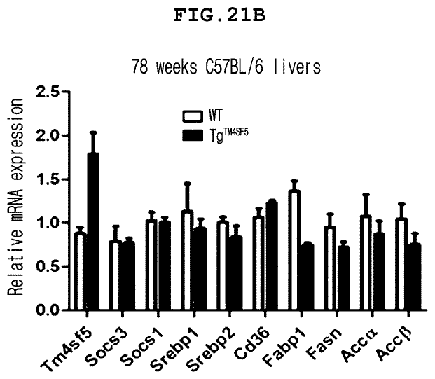

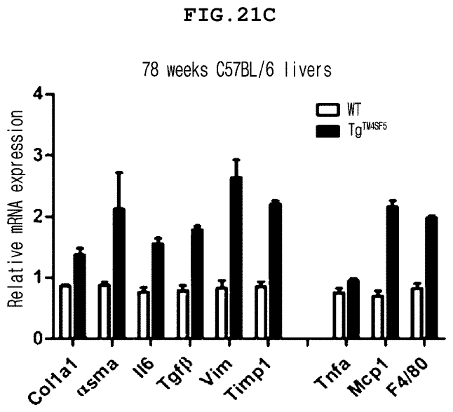

The expression of SREBP1, SREBP2, SREBP1c, SOCS1 or SOCS3 mRNA or protein in the liver tissue of the mouse (78 weeks old) transformed with a construct expressing TM4SF5 protein was not decreased or increased compared to that of the normal control group not expressing TM4SF5, the phosphorylation of STAT3 protein was increased, the levels of various factors related to fatty liver were similar to the levels present in the normal animal (without increasing), the mRNA levels of genes related to liver fibrosis and inflammation were increased, and the liver tissue exhibited the phenotype of liver fibrosis, liver cirrhosis or hepatitis (see FIGS. 20 and 21).

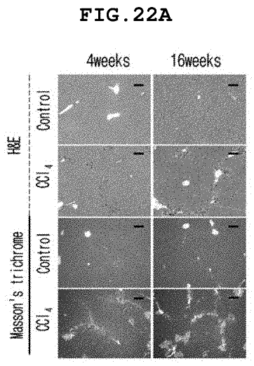

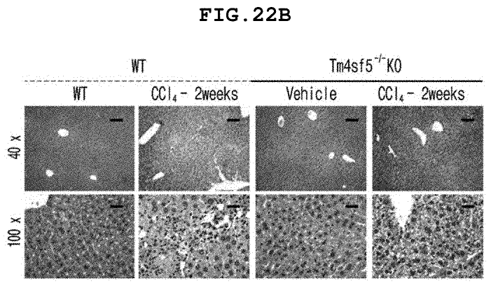

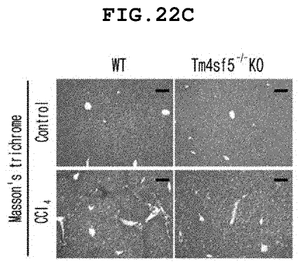

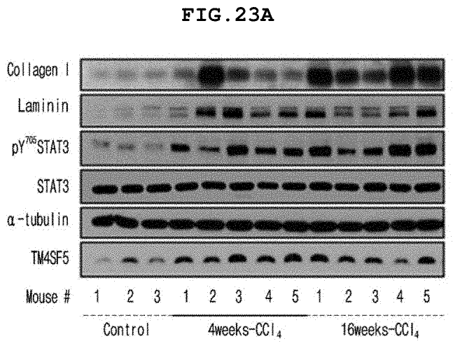

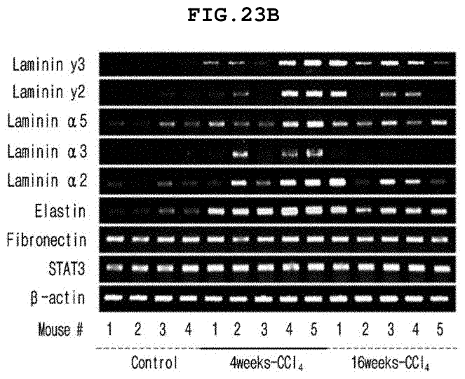

In addition, the present inventors constructed a liver disease animal model of liver fibrosis/liver cirrhosis by administering CCl₄ for 4 weeks or 16 weeks, according to the conventional method for preparing a liver disease animal model, and confirmed the liver tissue damage and the expression accumulation in the animal model (see FIG. 22). It was also confirmed that the expression of TM4SF5 protein and the phosphorylation of STAT3 protein were increased, and the expression of mRNAs and proteins of polypeptides (chains) constituting collagen and laminin was increased in the animal model (see FIG. 23).

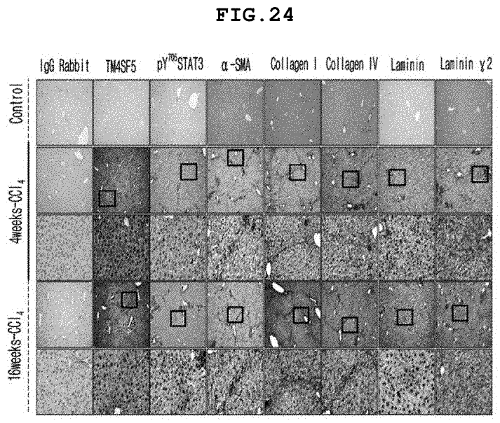

Through the liver tissue staining of the animals, it was confirmed that the expression of TM4SF5 and the expression of α-SMA, collagen I, laminin or laminin γ2 were increased, and the phosphorylation of STAT3, c-Src, FAK or Akt protein was correlatively increased in the liver disease animal model of liver fibrosis/liver cirrhosis and primary epithelial cells (see FIGS. 24, 25 and 26).

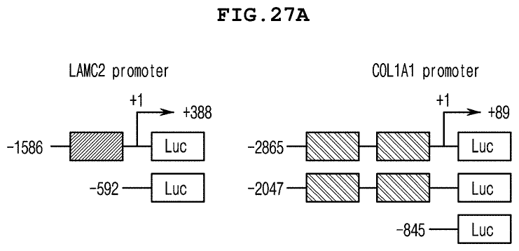

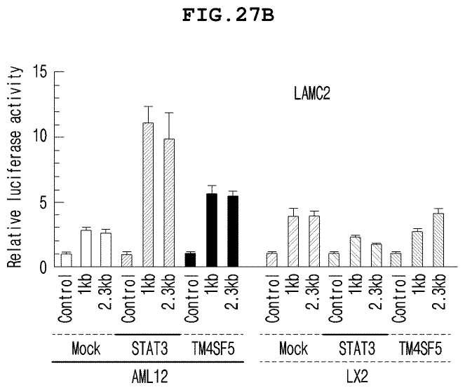

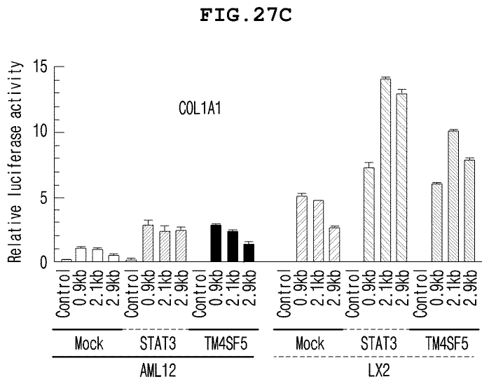

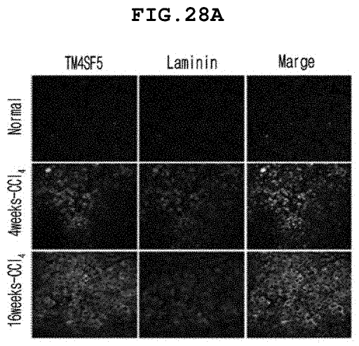

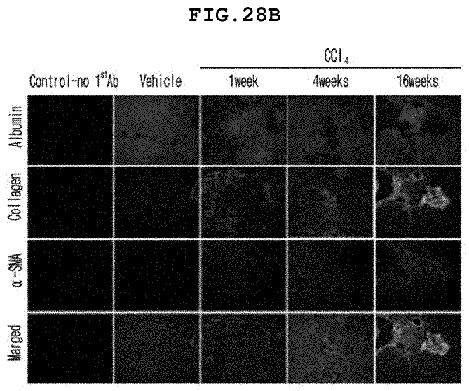

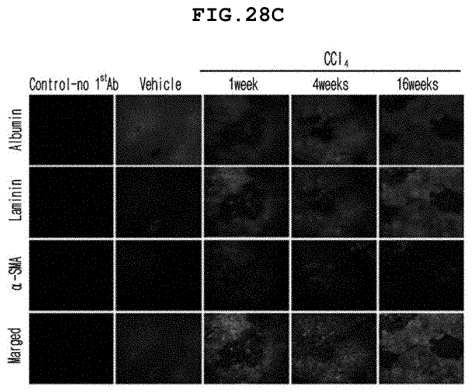

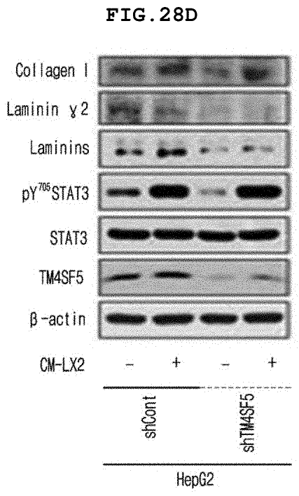

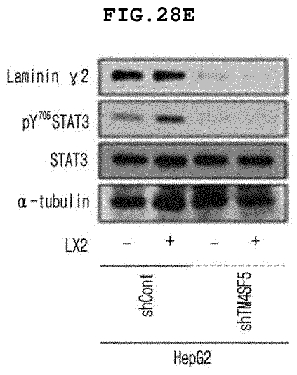

The present inventors confirmed that the expression of collagen in hepatic stellate cells and the expression of laminin in hepatic epithelial cells were regulated by the phosphorylation of STAT3 protein by binding to the promoters of collagen type I alpha 1 chain and laminin γ2 (see FIGS. 27 and 28).

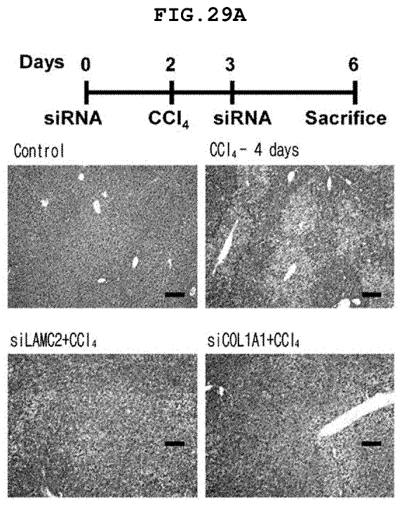

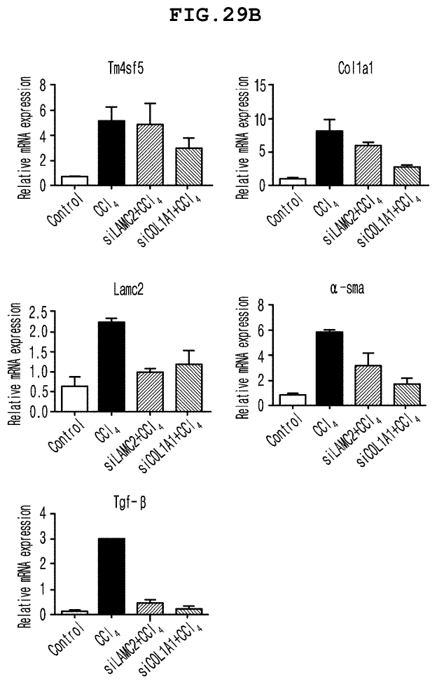

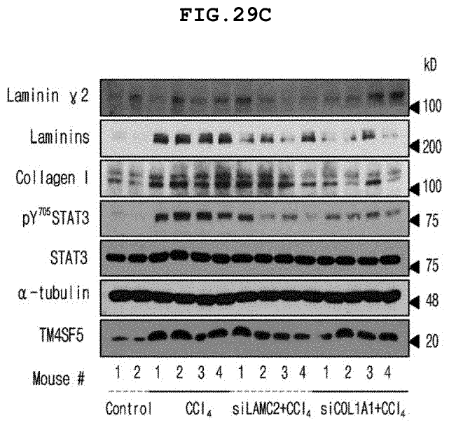

When the expression of laminin γ2 or collagen type I alpha 1 chain was suppressed in the normal animal and CCl₄ was treated to the animal, it was confirmed that the liver tissue damage was inhibited, the expression of TGFβ1, α-SMA, laminin or collagen was suppressed, the phosphorylation of STAT3 protein was inhibited, and the expression of laminin γ2 or collagen type I alpha 1 chain was important for liver fibrosis (see FIG. 29).

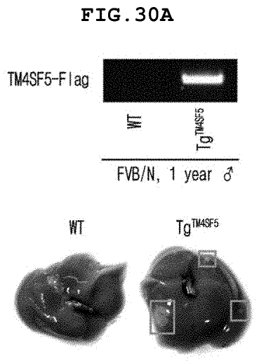

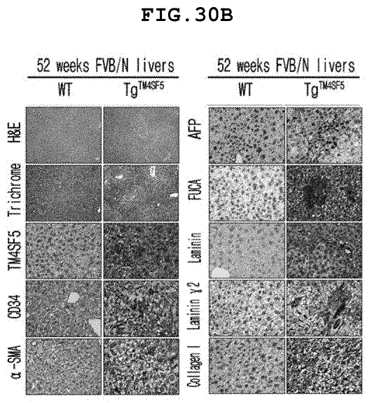

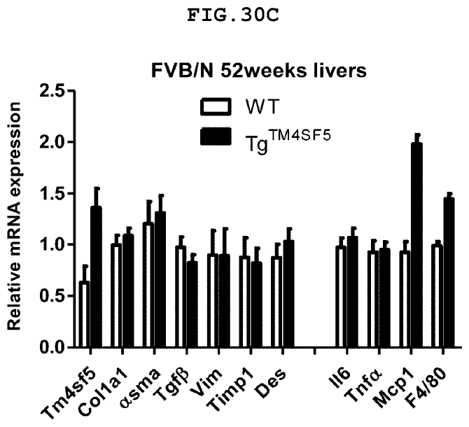

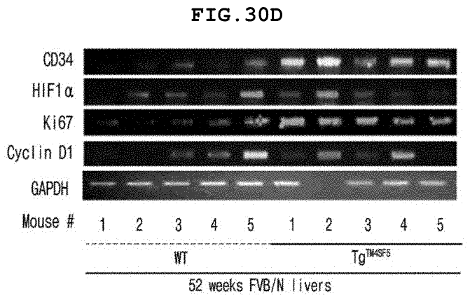

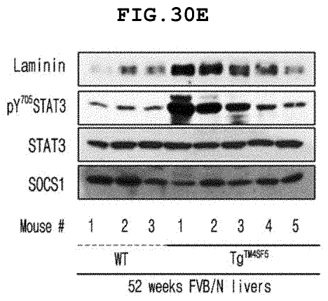

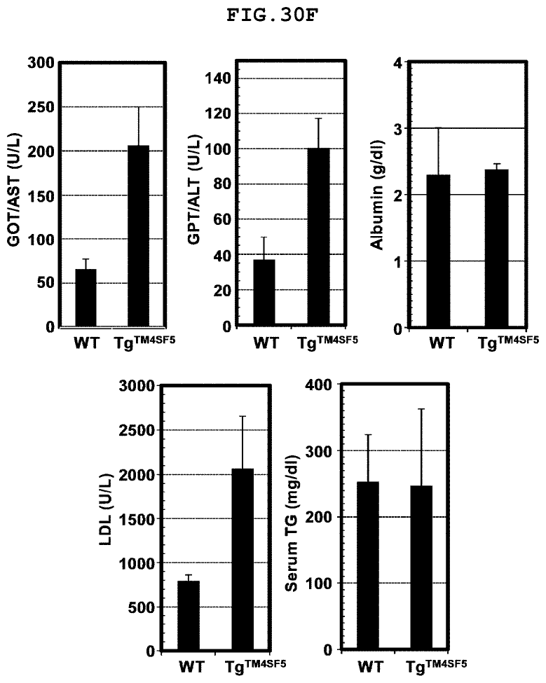

When Tm4sf5 gene was over-expressed in FVB/N animals, the nodules suggesting a tumor were confirmed in the liver tissue, the expression of CD34, α-SMA, AFP, FUCA, laminin, laminin γ2, collagen, MCP-1, F4/80 antigen, Hif1a, Ki67 or Cyclin D1 mRNA or protein was increased, and the level of AST, ALT, LDL or triglyceride (TG) in plasma was increased (see FIG. 30).

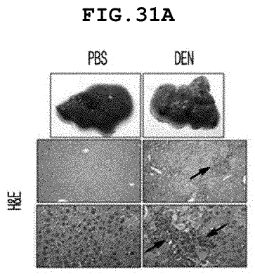

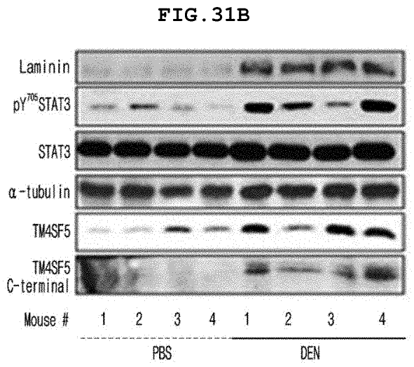

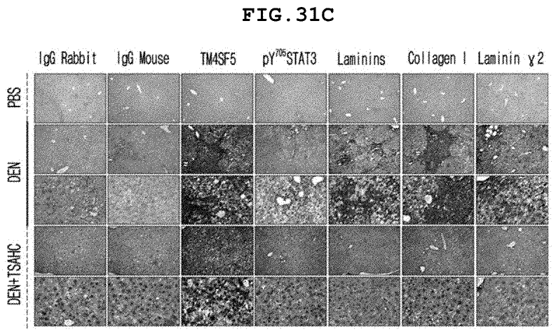

In the liver cancer model treated with DEN, it was confirmed that the nodule formation and the liver tissue damage were observed in the liver tissue, and the expression of TM4SF5, laminin, collagen or laminin γ2 was increased, and the phosphorylation of STAT3 protein was increased (see FIG. 31).

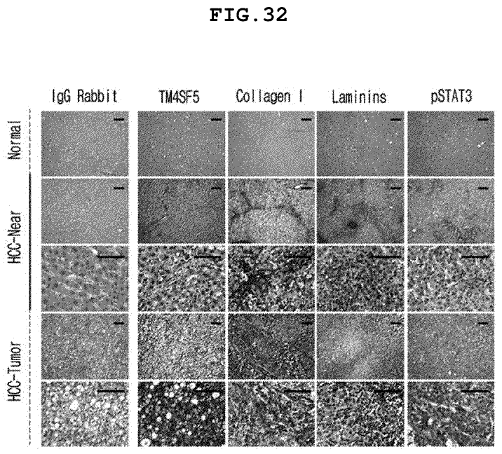

Therefore, when TM4SF5 protein is increased in the cancer region or the surrounding area of the liver tissue sample of a patient with suspected liver disease, the expression of SREBP1, SREBP2, SREBP1c, laminin or collagen mRNA or protein and the phosphorylation level of STAT3, c-Src, FAK or Akt protein are measured (see FIG. 32), which can used to provide information for the diagnosis of liver diseases.

The present invention also provides a method for screening a candidate substance for treating fatty liver:

1) treating a test substance to the cells expressing TM4SF5 and SREBP1 proteins;

2) measuring the expression level of SREBP1 mRNA or protein, and the phosphorylation level of one or more proteins selected from the group consisting of STAT3 protein, c-Src protein, FAK protein, mTOR, S6K, ULK, 4EBP1 and Akt proteins in the cells of step 1); and

3) selecting a test substance that suppresses the expression level of SREBP1 mRNA or protein and increases the phosphorylation level of one or more proteins selected from the group consisting of STAT3 protein, c-Src protein, FAK protein, mTOR, S6K, ULK, 4EBP1 and Akt proteins in the cells of step 1), or suppresses the expression level of SREBP1 mRNA or protein and reduces the synthesis of monoacyl-, diacyl- or triacyl-glycerol in step 2) compared to the control group not treated with the test substance.

The TM4SF5, SREBP1, SREBP2, Fasn, CD36, Fabp1, ApoB100, Pparα, Pparγ, Leptin, Accα, Accβ STAT3, collagen type I, laminin and laminin γ2 proteins have the characteristics as described above. For example, the TM4SF5, SREBP1 and STAT3 proteins may be any sequence well known in the art, and can include variants or fragments of the sequence. Specifically, the TM4SF5, SREBP1 and STAT3 proteins may be the polypeptides composed of the amino acid sequences represented by SEQ. ID. NO: 1, NO: 2 and NO: 3, respectively. In addition, The triglyceride, Vldlr, Ldlr and free fatty acid are the components of fatty acid and fat known in the art.

In the method for screening a candidate substance for treating fatty liver according to the present invention, the candidate substance capable of treating fatty liver can be screened by using the expression changes of TM4SF5, SREBP1, Srebp2, Fasn, CD36, Fabp1, ApoB100, Pparα, Pparγ, Leptin, Accα or Accβ protein, and the changes of the phosphorylation level of STAT3, c-Src, FAK (focal adhesion kinase), mTOR, S6K, ULK1, 4EBP1 or Akt protein in the cells expressing the proteins.

The method for screening a candidate substance for treating liver cancer according to the present invention can further include a step of confirming the increase of the expression of any one or more proteins selected from the group consisting of CD34, AFU, FUCA, laminin γ2, HIF1α and cyclin D1 together with the expression of TM4SF5 protein, or confirming the binding of TM4SF5 protein to mTOR, SLC7A1 or arginine. The candidate substance for treating liver disease including liver cancer selected by the screening method according to the present invention can inhibit the binding of the TM4SF5 protein to mTOR, SLC7A1 or arginine.

In a specific embodiment of the present invention, the present inventors prepared a transgenic mouse model expressing TM4SF5 protein, and confirmed that the formation of fat in the liver tissue of the mouse model was promoted to display the phenotype of fatty liver (see FIGS. 1 and 2).

In addition, it was confirmed that the expression of SREBP1, SREBP2, SREBP1c, CD36, Fabp1, Fasn, Accα, Accβ, Ldlr, SOCS1 or SOCS3 mRNA or protein was increased in the liver tissue of the transgenic mouse or the hepatocytes obtained from the liver tissue, the phosphorylation of STAT3 protein was decreased, and the levels of triglyceride (TG), AST and ALT were increased in the liver tissue (see FIGS. 2 and 3). The results were the same in the cell model over-expressing TM4SF5 protein (see FIG. 5).

Therefore, it was confirmed that a candidate substance for treating fatty liver can be screened by measuring the expression level of SREBP1, SREBP2, SREBP1c, CD36, Fabp1, Fasn, Accα, Accβ, Ldlr, SOCS1 or SOCS3 protein and the phosphorylation of STAT3, c-Src or FAK protein in the cells expressing TM4SF5 protein.

In a specific embodiment of the present invention, the present inventors prepared a transgenic mouse over-expressing TM4SF5 protein, and confirmed that the formation of fat was promoted in the transgenic mouse (see FIGS. 1 and 2), the weight gain of the TM4SF5 gene knockout mouse by the normal diet was lower than that of the normal mouse (see FIG. 9), and the weight gain of the knockout mouse by the high carbohydrate diet, high fat diet or high arginine was also lower than that of the normal mouse (see FIGS. 10, 11, 17 and 19).

The present invention also provides a method for screening a candidate substance for treating liver fibrosis, hepatitis or liver cirrhosis comprising the steps of treating a test substance to the cells expressing TM4SF5 protein and having phosphorylated STAT3 protein; measuring the expression level of SREBP1 protein and the phosphorylation level of any one or more proteins selected from the group consisting of STAT3, c-Src, FAK, mTOR, S6K, ULK, 4EBP1 and Akt in the cells; and selecting a test substance that increases the expression level of SREBP1 protein and suppresses the phosphorylation level of STAT3 protein compared to the control group not treated with the test substance.

The TM4SF5, SREBP1 and STAT3 proteins have the characteristics as described above. For example, the TM4SF5, SREBP1 and STAT3 proteins may be any sequence well known in the art, and can include variants or fragments of the sequence. Specifically, the TM4SF5 protein may be the polypeptide composed of the amino acid sequence represented by SEQ. ID. NO: 1.

In the method for screening a candidate substance for treating fatty liver according to the present invention, the candidate substance capable of treating liver fibrosis, hepatitis, liver cirrhosis or liver cancer can be screened by using the expression changes of SREBP1 protein, and the changes of the phosphorylation level of any one or more proteins selected from the group consisting of STAT3 protein, c-Src protein, FAK, mTOR, S6K, ULK, 4EBP1 or Akt protein in the cells expressing TM4SF5 and SREBP1 proteins.

The present invention also provides a method for screening a candidate substance for treating obesity, fatty liver or liver cancer comprising the following steps:

1) treating a test substance to the cells or the animal model expressing TM4SF5 protein;

2) measuring the binding of TM4SF5 protein to any one or more selected from the group consisting of mTOR protein, SLC7A1 protein and arginine in the cells or the animal model of step 1);

3) measuring the phosphorylation level of mTOR protein, S6K protein, UNC-51-like kinase 1 (ULK1) protein or 4EBP1 protein in the cells or the animal model of step 1);

4) measuring the level of monoacyl-, diacyl- or triacyl-glycerol in the cells or the animal model of step 1);

5) measuring any one or more selected from the group consisting of weight gain, glucose resistance, insulin resistance and glycolysis reactivity in the cells or the animal model of step 1);

6) measuring the expression levels of the genes related to glycolysis in the cells or the animal model of step 1); and

7) selecting a test substance that suppresses the binding of TM4SF5 protein to any one or more selected from the group consisting of mTOR protein, SLC7A1 protein and arginine in step 2), inhibits the phosphorylation of mTOR protein, S6K protein, UNC-51-like kinase 1 protein or 4EBP1 protein in step 3), reduces the level of monoacyl-, diacyl- or triacyl-glycerol in step 4), and decreases the weight gain, glucose resistance, insulin resistance or glycolysis reactivity in step 5).

The term “mTOR (mammalian target of rapamycin)” used in this specification means a hub signal transduction for the regulation of cellular metabolic functions (GenBank accession number: NM_004958.3). The term “SLC7A1 (solute carrier family 7 member 1) protein” means an arginine transporter present in the cell membrane and lysosomal membrane (GenBank accession number: NM_003045.4).

The TM4SF5 and SLC7A1 proteins have the characteristics as described above. For example, the TM4SF5 and SLC7A1 proteins may be any sequence well known in the art, and can include variants or fragments of the sequence. Specifically, the TM4SF5 and SLC7A1 proteins may be the polypeptides composed of the amino acid sequences represented by SEQ. ID. NO: 1 and NO: 2, respectively.

In the method for screening an anti-obesity candidate substance according to the present invention, the anti-obesity candidate substance and the liver cancer cell survival inhibitor candidate can be screened by selecting a test substance that inhibits the binding of the TM4SF5 protein to mTOR, SLC7A1 or arginine. The binding of the TM4SF5 protein to arginine can be mediated by the 124^{th} to 129^{th} residues from the N-terminus of the TM4SF5 protein.

In a specific embodiment of the present invention, the present inventors prepared a transgenic mouse over-expressing TM4SF5 protein, and confirmed that the formation of fat was promoted in the transgenic mouse (see FIGS. 1 and 2). The results were the same in the cells over-expressing TM4SF5 protein, and it was confirmed that TM4SF5 protein bound to mTOR, SLC7A1 and arginine, respectively, in the cells (see FIGS. 14 and 15).

Therefore, it was confirmed that the anti-obesity and anti-cancer candidates can be screened by measuring the inhibition of the binding of TM4SF5 protein to mTOR, SLC7A1 or arginine.

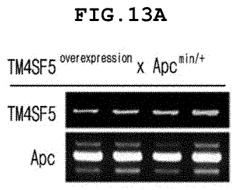

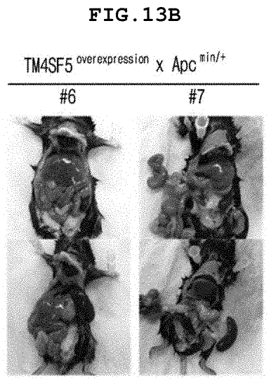

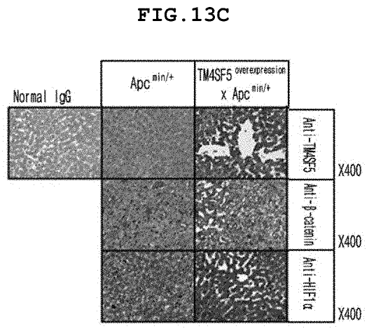

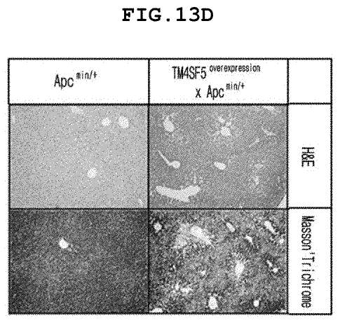

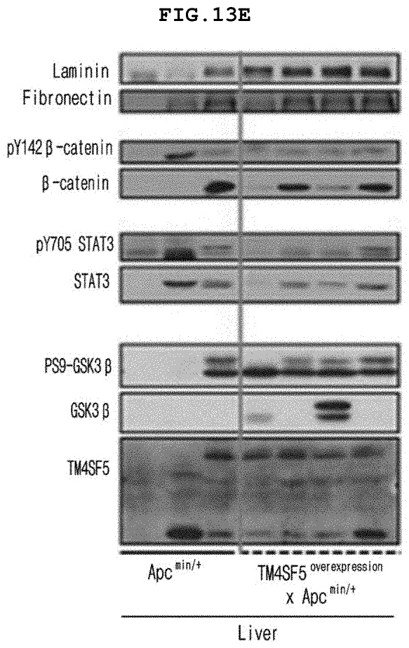

The present invention also provides a method for preparing a portal hypertension animal model comprising the step of mating a TM4SF5 knock-out (KO) mouse with a mouse having the genotype of APC^{min/+} (see FIG. 13).

As used herein, the “APC (adenomatous polyposis coli) gene” is a causative gene for familial colorectal adenomatosis, and the product synthesized from the said APC gene forms a complex with β-catenin to promote its degradation.

The TM4SF5 (GenBank Accession NO. NM_003963) and APC (GenBank Accession NO. M74088) genes can be the polynucleotides composed of any nucleotide sequences known in the art. The polynucleotide can be a polynucleotide composed of any nucleotide sequence encoding TM4SF5 protein. The TM4SF5 gene of the present invention can be a polynucleotide composed of the nucleotide sequence represented by SEQ. ID. NO: 3. The TM4SF5 gene may have 70%, 80%, 90%, 95% or 99% homology with the nucleotide sequence represented by SEQ. ID. NO: 3.

In a specific embodiment of the present invention, the inventors prepared a TM4SF5 gene knockout (KO) mouse, and then crossed the mouse with a mouse having the genotype of APC^{min/+} to obtain offspring (see FIG. 13A). It was confirmed that the offspring exhibited the symptoms of portal hypertension (see FIG. 13B).

Therefore, it was confirmed that an animal model of portal hypertension can be prepared by mating a TM4SF5 gene KO mouse and a mouse having the genotype of APC^{min/+}.

In addition, the present invention provides a portal hypertension animal model prepared by the above method.

The animal model can be prepared by the preparation method as described above. In one example, the preparation method can include a step of mating a TM4SF5 gene KO mouse with a mouse having the genotype of APC^{min/+}. At this time, the TM4SF5 and APC genes can have the characteristics as described above, and can include variants and fragments thereof. The TM4SF5 and APC genes can be the polynucleotides composed of the nucleotide sequences represented by SEQ. ID. NO: 3 and NO: 4, respectively.

In a specific embodiment of the present invention, the inventors prepared a portal hypertension animal model by mating a TM4SF5 knock-out (KO) mouse with a mouse having the genotype of APC^{min/+} (see FIGS. 13A and 13B).

Hereinafter, the present invention will be described in detail by the following examples.

However, the following examples are only for illustrating the present invention, and the contents of the present invention are not limited thereto.

### Example 1: Preparation of Transgenic Mouse Over-Expressing TM4SF5 Protein

### <1-1> Preparation of Transgenic Mouse Over-Expressing TM4SF5 Protein

In order to confirm the liver disease phenotype of the mouse over-expressing TM4SF5 protein, a transgenic mouse model was prepared in the following ways.

First, a construct wherein the Flag-labeled TM4SF5 protein (GenBank Accession NO. CAG33206) and BGH (bovine growth hormone) poly-A region (Macrogen, Korea) were expressed under the control of CMV promoter was constructed (J Cell Sci. 2012, 125(Pt 24):5960-73).

The prepared construct was injected into the fertilized egg of a C57BL/6 mouse using a microinjection method.

Two weeks after the injection, the liver tissue was obtained from the mouse and PCR was performed by the conventional method using the primers listed in Table 1 below (FIG. 1A), and the results are shown in FIG. 1B.

TABLE 1

| Name | Sequence (5′→3′) | SEQ. ID. NO: |
|---|---|---|
| CMV | CGCTATTACCATGGTGATGCG | SEQ. ID. NO: 5 |
| forward | | |
| TM4SF5 | AGACACCGAGAGGCAGTAGAT | SEQ. ID. NO: 6 |
| reverse | | |

As shown in FIG. 1B, the CMV promoter and TM4SF4 gene fragment of about 0.6 kb were detected, confirming that the TM4SF5 gene was inserted into the mouse (FIG. 1B).

### <1-2> Confirmation of Fatty Liver Phenotype in Transgenic Mouse Over-Expressing TM4SF5 Protein-1

The mice prepared in Example <1-1> were raised for 52 weeks, and then sacrificed to obtain the liver tissues. The appearance of the obtained liver tissue was observed, and the results are shown in FIG. 2A. At this time, the normal mouse was used as the control.

As shown in FIG. 2A, the mice over-expressing TM4SF5 protein raised for 52 weeks showed the characteristics of fatty liver (FIG. 2A).

### <1-3> Confirmation of Fatty Liver Phenotype in Transgenic Mouse Over-Expressing TM4SF5 Protein-2

H&E staining was performed using the liver tissue of the transgenic mouse over-expressing TM4SF5 protein obtained in Example <1-1>.

First, the dissected liver tissue was fixed to paraffin, and then slides were made. For H&E staining, the obtained liver tissue was left in a 60° C. oven for about 20 minutes to remove paraffin. The paraffin-removed liver tissue was immersed in xylene for 5 minutes, and this process was repeated 3 times. Next, the liver tissue was sequentially placed in 100%, 90%, 80% and 70% ethanol, and distilled water for 3 minutes each, and then taken out, followed by reaction in a hematoxylin solution for 5 minutes. Upon completion of the reaction, the liver tissue was washed with tap water, followed by reaction in an eosin solution for 20 minutes. The liver tissue was washed again with tap water, and then sequentially placed in 70%, 80%, 90% and 100% ethanol, and a xylene solution for 3 minutes each, and then placed on a slide and mounted. The slide glass was observed using a microscope and the results are shown in FIG. 2B.

As shown in FIG. 2B, it was confirmed that fat was accumulated in the liver tissue of the transgenic mouse over-expressing TM4SF5 protein (FIG. 2B).

### <1-4> Confirmation of Fatty Liver Phenotype in Transgenic Mouse Over-Expressing TM4SF5 Protein-3

Oil red 0 staining was performed using the liver tissue of the transgenic mouse over-expressing TM4SF5 protein obtained in Example <1-1> in the following ways.

First, blood of the transgenic mouse prepared in Example <1-1> was removed by adding a perfusate, and hepatocytes were separated using type 2 collagen. The isolated hepatocytes were filtered using a cell filter having a pore size of 40 μm, and centrifugation was performed to obtain pellets. The obtained pellets were cultured using the William's E medium supplemented with 1% penicillin/streptomycin and 10% FBS. At this time, the culture was performed using a plate coated with collagen.

The cultured hepatocytes were put in 10% formalin, fixed for 15 minutes, and washed with PBS. Meanwhile, the oil red 0 dye (Sigma, Germany) was mixed with sterile distilled water to prepare a mixed solution, and the prepared mixed solution was filtered. The filtered oil red 0 solution was added to the washed cells, which were stained for 30 minutes, followed by washing with distilled water. The stained cells were observed using a microscope, and the results are shown in FIG. 2B.

As shown in FIG. 2B, it was confirmed that fat was accumulated in the hepatocytes obtained from the transgenic mouse over-expressing TM4SF5 protein (FIGS. 2B and 2C).

### <1-5> Confirmation of Fatty Liver Phenotype in Transgenic Mouse Over-Expressing TM4SF5 Protein-4

The levels of triglyceride (TG), albumin and ALT in the blood of the transgenic mouse over-expressing TM4SF5 protein obtained in Example <1-1> were measured in the following ways.

First, blood was obtained before sacrificing the transgenic mouse. The obtained blood was placed in a 1.5 ml tube coated with 1 M EDTA, and 8 μl of 1 M EDTA was added thereto. Serum was separated by centrifuging the tube at 1,500×g and 4° C. for 15 minutes. The levels of triglycerides, albumin and ALT were confirmed from the separated serum using a blood analyzer (Drichem 4000, Fuji, Japan).

As a result, as shown in FIG. 2D, the levels of triglyceride and ALT were increased in the liver tissue of the transgenic mouse over-expressing TM4SF5 protein compared to the normal mouse, but the level of albumin was not changed (FIG. 2D). From the above results, it was confirmed that the liver tissue of the transgenic mouse over-expressing TM4SF5 protein was damaged.

### Example 2: Confirmation of Changes of Signal Transduction Mechanism in Transgenic Mouse Over-Expressing TM4SF5 Protein

### <2-1> Confirmation of Expression Changes of Fatty Liver-Related Genes in Transgenic Mouse Over-Expressing TM4SF5 Protein

The expression changes of the fatty liver-related genes in the liver tissue of the transgenic mouse prepared in Example <1-1> were confirmed by the following method.

First, cells were lysed by adding Qiazol (Qiagen, USA) to the obtained liver tissue, and chloroform was added thereto, followed by centrifugation at 12,000×g and at 4° C. for 15 minutes. After the centrifugation, RNA was precipitated by adding isopropanol to the obtained supernatant. The precipitated RNA was washed with 70% ethanol, and centrifuged for 5 minutes under the conditions of 7,500×g and 4° C. to obtain RNA pellets. The RNA pellets were dried at room temperature for 10 minutes. RNA was obtained by adding 30 μl of DEPC-distilled water to the dried pellets.

From the obtained RNA, gDNA was removed and cDNA was obtained using a reverse transcription kit (Toyobo, Japan) according to the manufacturer's protocol. Real-time PCR was performed by adding 2× evergreen master mix (Labopass, Korea) and 0.4 μM of forward and reverse primers listed in Table 2 below to the obtained cDNA. From the PCR, the expression level of each gene was obtained using the modified Pfaffl delta-delta Ct method.

TABLE 2

| Name | Sequence (5′→3′) | SEQ. ID. NO: |
|---|---|---|
| Srebp1_F | CATCGACTACATCCGCTTCTT | SEQ. ID. NO: 7 |
| Srebp1_R | CACCAGGTCCTTCAGTGATTT | SEQ. ID. NO: 8 |
| Srebp2_F | TGGATGACGCAAAGGTCAA | SEQ. ID. NO: 9 |
| Srebp2_R | CAGGAAGGTGAGGACACATAAG | SEQ. ID. NO: 10 |
| Cd36 F | TTGGCCAAGCTATTGCGACA | SEQ. ID. NO: 11 |
| Cd36 R | CTGGAGGGGTGATGCAAAGG | SEQ. ID. NO: 12 |
| Fabp1_F | CCCGAGGACCTCATCCAGAA | SEQ. ID. NO: 13 |
| Fabp1_R | CCCCAGGGTGAACTCATTGC | SEQ. ID. NO: 14 |
| Fasn_F | TCTGGGCCAACCTCATTGGT | SEQ. ID. NO: 15 |
| Fasn_R | GAAGCTGGGGGTCCATTGTG | SEQ. ID. NO: 16 |
| Accα_F | ACATTCCGAGCAAGGGATAAG | SEQ. ID. NO: 17 |
| Accα_R | GGGATGGCAGTAAGGTCAAA | SEQ. ID. NO: 18 |
| Accβ_F | GTCCTGCCCACTTTCTTCTATC | SEQ. ID. NO: 19 |
| Accβ_R | GTTTAGCTCGTAGGCGATGTAG | SEQ. ID. NO: 20 |

As a result, as shown in FIG. 3A, the expressions of Srebp 1, Srebp 2, Cd36, Fabp1, Fasn, Accα, Accβ and Ldlr, the fatty liver-related genes, were increased in the liver tissue of the transgenic mouse over-expressing TM4SF5 protein (FIG. 3A).

### <2-2> Confirmation of Expression Changes of Fatty Liver-Related Proteins in Transgenic Mouse Over-Expressing TM4SF5 Protein

The expression changes of the fatty liver-related proteins in the liver tissue of the transgenic mouse prepared in Example <1-1> were confirmed by Western blotting.

Particularly, a lysis buffer [50 mM Tris-HCl (pH 7.4), 1% NP40, 0.25% sodium dioxycholate, 150 mM NaCl, 1 mM EDTA], SDS (sodium dodecyl sulfate), Na3O4V and protease inhibitor cocktail (GenDepot) were added to the obtained liver tissue, which was left at 4° C. for 15 minutes to lyse the tissue. The lysate was centrifuged for 30 minutes under the conditions of 13,000 rpm and 4° C. to obtain a supernatant. The proteins present in the supernatant were quantified using BCA reagent (Thermo Scientifics). 4× sample buffer [4 ml of 100% glycerol, 2.4 ml of Tris-HCl (pH 6.8), 0.8 g of SDS, 4 mg of brominated phenol blue, 0.4 ml of β-mercaptoethanol and 3.1 ml of H₂O, final volume: 10 ml] was added thereto, which was boiled at 100° C. for 5 minutes. SDS-PAGE was performed, and the proteins were transferred to a nitrocellulose membrane (Whatman). The membrane was pretreated in a solution containing 5% skim milk for 1 hour, and reacted with the antibodies against laminin (Abcam, UK), ACC1 (Cell Signalling, USA), SREBP1 precursor (Santa cruz, USA), mature SREBP1 (Santa cruz, USA), MTP (Santa cruz, USA), PPARα (Santa cruz, USA), pY⁷⁰⁶STAT3 (Millipore, USA), STAT3 (Santa cruz, USA), α-tubulin (Sigma, USA) and TM4SF5 (J Clin Invest. 2008 April; 118(4): 1354-66) as the primary antibodies at 4° C. for 15 hours. Then, the membrane was reacted with the secondary antibody, and developed on an X-ray film using an ECL solution (Pierce, USA). The results are shown in FIG. 3B.

As shown in FIG. 3B, the expressions of SREBP1 and ACC1 (ACC.) proteins, the fatty liver-related proteins, were significantly increased in the liver tissue of the transgenic mouse over-expressing TM4SF5 protein, but the phosphorylation of STAT3 protein was suppressed (FIG. 3B).

### <2-3> Confirmation of Suppression of STAT3 Protein Phosphorylation in Transgenic Mouse Over-Expressing

TM4SF5 Protein The suppression of the STAT3 protein phosphorylation in the transgenic mouse over-expressing TM4SF5 protein, confirmed in Example <2-2>, was confirmed again by using histostaining.

The obtained liver tissue was left in a 60° C. oven for about 20 minutes to remove paraffin. The paraffin-removed liver tissue was immersed in xylene for 5 minutes, and this process was repeated 3 times. Next, the liver tissue was sequentially placed in 100%, 90%, 80% and 70% ethanol, and distilled water for 3 minutes each, and in tap water for 10 minutes. The liver tissue was put in 10 mM citric acid buffer (pH 6.0), and covered with foil, which was autoclaved. Upon completion of the autoclave, the tissue was sufficiently cooled, reacted in PBS for 10 minutes twice, and 3% hydrogen peroxide was made using methanol to undergo a step of quenching for 15 minutes. This was put in PBS again and reacted three times for 5 minutes each. Then, blocking was performed in PBS containing 5% horse or goat serum at 4° C. for one day. On the next day, the tissue was reacted three times with PBS for 5 minutes each, and reacted with biotin-conjugated IgG rabbit or mouse for 1 hour using the serum used for the primary reaction. The tissue was washed again with PBS, and reacted with an avidin-biotin-peroxidase complex prepared in advance for 30 minutes. The tissue was washed 3 times with PBS for 5 minutes each and stained with DAB. At this time, the reaction time was different according to the antibody used, so the time point was determined in comparison with the control group. The tissue stained with DAB was placed in distilled water and reacted with hematoxylin for more than 5 minutes. The liver tissue was washed with tap water, and then sequentially placed in 70%, 80%, 90% and 100% ethanol, and a xylene solution for 3 minutes each, and then placed on a slide and mounted.

As shown in FIG. 3C, the expression of SREBP1 was increased and the phosphorylation of STAT3 protein was suppressed in the liver tissue of the transgenic mouse over-expressing TM4SF5 protein, compared to the control group (FIG. 3C).

### Example 3: Confirmation of Signal Transduction Mechanism Change in Hepatocytes Over-Expressing TM4SF5 Protein

### <3-1> Confirmation of Fat Accumulation in Hepatocytes Over-Expressing TM4SF5 Protein

The above results were reconfirmed using the hepatocytes over-expressing TM4SF5 protein.

First, hepatocytes were obtained under the same conditions and methods as described in Example <1-4>, except that the C57BL/6 normal mouse was used instead of the transgenic mouse over-expressing TM4SF5 protein. The obtained hepatocytes were transformed with the construct containing TM4SF5 gene prepared in Example <1-1>. Oil red 0 staining was performed using the cells transformed with the construct expressing TM4SF5 under the same conditions and methods as described in Example <1-4>. At this time, the hepatocytes obtained from the normal mouse and treated with fatty acids (FFA) were used as the positive control. The stained cells were observed using a microscope, and the results are shown in FIG. 4A.

As shown in FIG. 4A, it was confirmed that fat was accumulated in the hepatocytes over-expressing TM4SF5 protein (FIG. 4A).

### 3-2. Confirmation of Expression Changes of Fat-Related Genes in Cells Over-Expressing TM4SF5 Protein

The expression changes of the fat-related genes were confirmed using the hepatocytes expressing TM4SF5 protein prepared in Example <3-1>. At this time, the hepatocytes over-expressing or not-expressing TM4SF5 protein treated with free fatty acid, or the hepatocytes expressing TM4SF5 protein, the normal hepatocytes treated with IL-6, a cytokine associated with fatty liver, and the hepatocytes expressing TM4SF5 protein treated with IL-6 were used for the comparison. The experiment was performed under the same conditions and methods as described in Example <2-1>, except that the primers listed in Table 3 were used.

TABLE 3

| Name | Sequence (5′→3′) | SEQ. ID. NO: |
|---|---|---|
| Tm4sf5_F | GTCTTCTCCTCCGCCTTTG | SEQ. ID. NO: 21 |
| Tm4sf5_R | GGTAGTCCCACTTGTTGTCTATT | SEQ. ID. NO: 22 |
| Srebp2_F | TGGATGACGCAAAGGTCAA | SEQ. ID. NO: 23 |
| Srebp2_R | CAGGAAGGTGAGGACACATAAG | SEQ. ID. NO: 24 |
| Cd36_F | TTGGCCAAGCTATTGCGACA | SEQ. ID. NO: 25 |
| Cd36_R | CTGGAGGGGTGATGCAAAGG | SEQ. ID. NO: 26 |
| Fabp1_F | CCCGAGGACCTCATCCAGAA | SEQ. ID. NO: 27 |
| Fabp1_R | CCCCAGGGTGAACTCATTGC | SEQ. ID. NO: 28 |
| Fasn_F | TCTGGGCCAACCTCATTGGT | SEQ. ID. NO: 29 |
| Fasn_R | GAAGCTGGGGGTCCATTGTG | SEQ. ID. NO: 30 |
| Ldlr_F | GCCTTTGCCAAAACGTCACC | SEQ. ID. NO: 31 |
| Ldlr_R | CCTGAGGTCCCATCCAATGC | SEQ. ID. NO: 32 |
| Vldlr_F | TCAGTCCCAGGCAGCGTAT | SEQ. ID. NO: 33 |
| Vldlr_R | CTTGATCTTGGCGGGTGTT | SEQ. ID. NO: 34 |

As a result, as shown in FIGS. 4B and 4C, the expressions of Srebp1, Srebp2, Fasn, CD36, Fabp1, Vldlr and Ldlr, the fat-related genes, were increased in the hepatocytes over-expressing TM4SF5 protein and the normal hepatocytes treated with IL-6 (FIGS. 4B and 4C). In FIG. 4B, cont+FFA means the control cells treated with free fatty acid (250 μM steric acid+250 μM palmitic acid). In the case of knockout mouse in which Tm4sf5 gene was removed as a heterozygote (Tm4sf5-/+), the levels of ApoB100, Ldlr, Srebp2, Pparγ and Leptin, the genes related to fat biosynthesis and transport accumulation, were kept low, unlike the normal mouse (FIG. 4D).

### <3-3> Confirmation of Suppression of STAT3 Protein Phosphorylation in Cells Over-Expressing TM4SF5 Protein

The expression changes of the fatty liver related proteins in the cells over-expressing TM4SF5 protein were confirmed by Western blotting. The experiment was performed under the same conditions and methods as described in Example <2-2>, except that the antibodies against laminin, SREBP1 precursor, mature SREBP1, PPARγ, pY⁷⁰⁵STAT3, STAT3, β-actin and Flag were used as the primary antibodies.

As a result, as shown in FIG. 5A, the expression of SREBP1 protein was increased, but the phosphorylation of STAT3 protein was decreased in the cells over-expressing TM4SF5 protein, similar to the normal hepatocytes treated with FFA (FIG. 5A).

To confirm whether the increase of the SREBP1 protein expression and the decrease of the STAT3 protein phosphorylation have a competitive relationship, Western blotting was performed in the same manner as described above using the hepatocytes over-expressing STAT3 protein treated with fatty acid. As a result, as shown in FIG. 5B, the increase of the SREBP1 protein expression was suppressed when the expression of STAT3 protein was over-expressed (FIG. 5B).

Meanwhile, after over-expressing SREBP1 protein in the normal hepatocytes, the phosphorylation of STAT3 protein was confirmed by the same method as above. As a result, as shown in FIG. 5C, the phosphorylation of STAT3 protein was significantly reduced by the increased expression level of SREBP1 protein (FIG. 5C).

Therefore, it was confirmed from the above results that the expression level of SREBP1 protein and the phosphorylation of STAT3 protein played opposite roles.

### Example 4: Confirmation of Signal Transduction Mechanism Change in Adipocytes with Suppressed TM4SF5 Protein Expression

<4-1> Confirmation of Fat Production Inhibition in Adipocytes with Suppressed TM4SF5 Protein Expression

To confirm whether the fat production was suppressed when the expression of TM4SF5 protein was suppressed in adipocytes, oil red O staining was performed.

First, mouse 3T3-L1 adipocyte progenitor cells were prepared by culturing in DMEM supplemented with 10% NBCS (Gibco, 16010159) and 1% penicillin/streptomycin. The prepared cells were distributed in a 6-well plate at the density of 1×10⁵/well.

After 4 days of the distribution, when the progenitor cells were full in the wells, the cells were further cultured for 48 hours. Then, the medium was replaced with the adipocyte differentiation medium (MDI medium containing 10% FBS) supplemented with 1 μM dexamethasone, 0.5 mM IBMX (3-Isobutyl-1-methylxanthine) and 10 μg/m [symbol] of insulin (Sigma, USA). After culturing the cells for 2 days, the medium was replaced with DMEM supplemented with 10% FBS and 10 μg/m [symbol] of insulin. After replacing with the medium, the cells were cultured for 10 days, and then cultured using DMEM supplemented with 10% FBS and 1% penicillin/streptomycin to obtain differentiated adipocytes. The adipocytes were transfected with TM4SF5 shRNA (shTM4SF5, 5′-CCTGGAATGTGACGCTCTTCTCGCTGCTG-3′, SEQ. ID. NO: 35) using lipofectamine 3000.

As a result, as shown in FIG. 6A, the production of fat was inhibited when the expression of TM4SF5 gene was suppressed in adipocytes (FIG. 6A).

<4-2> Confirmation of Expression Change of Fat-Related Gene in Adipocytes with Suppressed TM4SF5 Protein Expression

Whether the expression of the fat-related gene changed when the expression of TM4SF5 gene was suppressed was confirmed by the following method. The experiment was performed under the same conditions and methods as described in Example <2-1>, except that the differentiated adipocytes obtained in Example <4-1> were treated with shRNA against TM4SF5, and then the primers listed in Table 4 were used.

TABLE 4

| Name | Sequence (5′→3′) | SEQ. ID. NO: |
|---|---|---|
| Tm4sf5_F | GTCTTCTCCTCCGCCTTTG | SEQ. ID. NO: 36 |
| Tm4sf5_R | GGTAGTCCCACTTGTTGTCTATT | SEQ. ID. NO: 37 |
| Srebp1 F | CATCGACTACATCCGCTTCTT | SEQ. ID. NO: 38 |
| Srebp1_R | CACCAGGTCCTTCAGTGATTT | SEQ. ID. NO: 39 |
| Cd36_F | TTGGCCAAGCTATTGCGACA | SEQ. ID. NO: 40 |
| Cd36_R | CTGGAGGGGTGATGCAAAGG | SEQ. ID. NO: 41 |
| Fabp1_F | CCCGAGGACCTCATCCAGAA | SEQ. ID. NO: 42 |
| Fabp1_R | CCCCAGGGTGAACTCATTGC | SEQ. ID. NO: 43 |
| Fasn_F | TCTGGGCCAACCTCATTGGT | SEQ. ID. NO: 44 |
| Fasn_R | GAAGCTGGGGGTCCATTGTG | SEQ. ID. NO: 45 |
| Pparγ_F | CTGGCCTCCCTGATGAATAAAG | SEQ. ID. NO: 46 |
| Pparγ_R | AGGCTCCATAAAGTCACCAAAG | SEQ. ID. NO: 47 |

As a result, as shown in FIG. 6B, the expressions of Pparγ, CD36, Fasn, Srebp1 and Fabp1 genes related to fat were suppressed when the expression of TM4SF5 gene was suppressed in adipocytes (FIG. 6B).

<4-3> Confirmation of Expression Change of Fat-Related Gene in Differentiation Process of Adipocytes with Increased TM4SF5 Protein Expression

Mouse 313-L1 preadipocytes were cultured in DMEM supplemented with 10% NBCS (Gibco, 16010159) and 1% penicillin/streptomycin. On the 4^{th} day of culture, if the preadipocytes were filled with 100% in the culture vessel, the cells were further cultured for hours, and then treated with the adipocyte differentiation medium (MDI medium) supplemented with 1 μM dexamethasone, 0.5 mM IBMX (3-Isobutyl-1-methylxanthine), 10 μg/ml of insulin (Sigma, USA) and 10% FBS for 2 days. Thereafter, the medium was replaced with DMEM supplemented with 10% FBS and insulin (10 μg/ml) for 2 days. On the 10^{th} day of culture, the cells were cultured with DMEM supplemented with 10% NBCS and 1% penicillin/streptomycin to differentiate into adipocytes.

At this time, whether the expression of the fat-related gene was changed in addition to the expression of TM4SF5 gene during the process of accumulating fat was confirmed by performing the experiment under the same conditions and methods as described in Example <2-2> using the primary antibodies against SREBP1 precursor, mature SREBP1, PPARγ, pY⁷⁰⁵STAT3, STAT3, β-actin (Cell Signaling Technology, USA), ERK (Cell Signaling Technology, USA), p-ERK (Cell Signaling Technology, USA), Akt (Cell Signaling Technology, USA) and TM4SF5.

As a result, as shown in FIG. 6C, the expressions of fat-related proteins were gradually increased, but the phosphorylation of STAT3 protein was gradually decreased as the adipocytes with increased TM4SF5 protein expression were differentiated (FIG. 6C).

### Example 5: Confirmation of Mechanisms of SREBP1 Protein Expression Increase and STAT3 Protein Phosphorylation Inhibition in Transgenic Mouse Over-Expressing TM4SF5 Protein

### <5-1> Confirmation of Mechanism of SREBP1 Protein Expression Increase in Transgenic Mouse Over-Expressing TM4SF5 Protein

The effect of the increased expression of SREBP1 protein by the over-expression of TM4SF5 protein on the expression patterns of SIRT genes, the factors that regulate the expression of SREBP1 protein, was investigated. The experiment was performed under the same conditions and methods as described in Example <2-1>, except that the primers listed in Table 5 below were used.

TABLE 5

| Name | Sequence (5′→3′) | SEQ. ID. NO: |
|---|---|---|
| Sirt1_F | GCATAGATACCGTCTCTTGATCTGAA | SEQ. ID. NO: 48 |
| Sirt11_R | TGTGAAGTTACTGCAGGAGTGTAAA | SEQ. ID. NO: 49 |
| Sirt2_F | TTCCATCGCGCTTCTTCTCC | SEQ. ID. NO: 50 |
| Sirt2_R | CCAGGCCACGTCCCTGTAAG | SEQ. ID. NO: 51 |
| Sirt3_F | ACCTCCTGGGGTGGACACAA | SEQ. ID. NO: 52 |
| Sirt3_R | GGCCCCAAGGGTAGACATCC | SEQ. ID. NO: 53 |
| Sirt4_F | AGCTTTCAGGTCCCGTGCTG | SEQ. ID. NO: 54 |
| Sirt4_R | TCAGGCAAGCCAAATCGTCA | SEQ. ID. NO: 55 |
| Sirt5_F | TCTACCCGGCTGCCATGTTT | SEQ. ID. NO: 56 |
| Sirt5_R | TGAGGAGCAAGGGCTTCAGG | SEQ. ID. NO: 57 |
| Sirt6_F | GGGACCTGATGCTCGCTGAT | SEQ. ID. NO: 58 |
| Sirt6_R | CAGAGGTGGCAGGGCTTTGT | SEQ. ID. NO: 59 |
| Sirt7_F | TGCCAGGCACTTGGTTGTCT | SEQ. ID. NO: 60 |
| Sirt7_R | TAGGCTCCGCTTCGCTTAGG | SEQ. ID. NO: 61 |

As a result, as shown in FIG. 7A, the expressions of SIRT1, SIRT5 and SIRT6 genes were decreased, but the expressions of SIRT2, SIRT4 and SIRT7 genes were increased in the liver tissue of the transgenic mouse over-expressing TM4SF5 protein (FIG. 7A).

### <5-2> Confirmation of Mechanism of STAT3 Protein Phosphorylation Inhibition in Transgenic Mouse Over-Expressing TM4SF5 Protein

The effect of the STAT3 protein phosphorylation inhibition by the over-expression of TM4SF5 protein on the expression patterns of SOCS genes, the factors that suppress STAT3 protein, was investigated. The experiment was performed under the same conditions and methods as described in Example <2-1>, except that the primers listed in Table 6 below were used.

TABLE 6

| Name | Sequence (5′→3′) | SEQ. ID. NO: |
|---|---|---|
| SOCS1_F | GGGTGGCAAAGAAAAGGAG | SEQ. ID. NO: 62 |
| SOCS1_R | GTTGAGCGTCAAGACCCAGT | SEQ. ID. NO: 63 |
| SOCS2_F | TCCAGATGTGCAAGGATAAACG | SEQ. ID. NO: 64 |
| SOCS2_R | AGGTACAGGTGAACAGTCCCATT | SEQ. ID. NO: 65 |
| SCOS3_F | TGCAGGAGAGCGGATTCTA | SEQ. ID. NO: 66 |
| SCOS3_R | AGCTGTCGCGGATAAGAAAG | SEQ. ID. NO: 67 |
| SCOS5_F | GAGGGAGGAAGCCGTAATGAG | SEQ. ID. NO: 68 |
| SCOS5_R | CGGCACAGTTTTGGTTCCG | SEQ. ID. NO: 69 |

As a result, as shown in FIG. 7C, the expressions of SOCS1 and SOCS3 genes were increased in the liver tissue of the transgenic mouse over-expressing TM4SF5 protein (FIG. 7C).

### <5-3> Confirmation of Mechanisms of SREBP1 Protein Expression Increase and STAT3 Protein Phosphorylation Inhibition in Transgenic Mouse Over-Expressing TM4SF5 Protein

The effect of the increased expression of SREBP1 protein and the STAT3 protein phosphorylation inhibition by the over-expression of TM4SF5 protein on the expression patterns of SIRT and SOCS proteins was investigated. The experiment was performed under the same conditions and methods as described in Example <2-2>, except that SCOS1 (Cell Signaling, USA), SOCS3 (Santa cruz, USA), SIRT1 (Santa cruz, USA) and β-tubulin were used as the primary antibodies.

As a result, as shown in FIG. 7B, the expressions of SOCS1 and SOCS3 proteins were increased, but the expression of SIRT1 protein was decreased in the liver tissue of the transgenic mouse over-expressing TM4SF5 protein (FIG. 7B).

In addition, a culture medium in which AML12 cells, the normal hepatocytes transformed with a construct expressing TM4SF5 protein, were cultured was obtained on the 4^{th}, 8^{th}, and 12^{th} days of culture, and 3T3-L1 cells were cultured in the obtained culture medium. The expression changes of SOCS3 protein in the cultured 313-L1 cells were confirmed by Western blotting in the same manner as above.

As a result, as shown in FIG. 7D, the expression level of SOCS3 protein was increased when the hepatic epithelial cells expressing TM4SF5 protein were cultured in the culture medium in which adipocyte progenitor cells were cultured (FIG. 7D).

### Example 6: Confirmation of Mechanisms of SREBP1 Protein Expression Increase and STAT3 Protein Phosphorylation Inhibition in Hepatocytes Over-Expressing TM4SF5 Protein

The effect of the increased expression of SREBP1 protein and the STAT3 protein phosphorylation inhibition by the over-expression of TM4SF5 protein on the expression patterns of SIRT and SOCS proteins in the hepatocytes separated from the normal mouse was investigated.

First, the hepatocytes over-expressing TM4SF5 protein were prepared under the same conditions and methods as described in Example <3-1>. The expression changes of SOCS1 and SOCS3 genes were confirmed using the prepared hepatocytes under the same conditions and methods as described in Example <2-1>, except that the primers listed in Table 3 were used. As a result, as shown in FIG. 8A, the expressions of SOCS1 and SOCS3 genes were increased by the over-expressed TM4SF5 protein, which was similar to the results when fatty acid was added (FIG. 8A).

In addition, the expression changes of SOCS1 and SOCS3 proteins in the hepatocytes were confirmed by Western blotting. As a result, as shown in FIG. 8B, the expressions of SOCS1 and SOCS3 proteins were increased in the hepatocytes over-expressing TM4SF5 protein compared to the control group (FIG. 8B).

The expression changes of SOCS1 and SOCS3 proteins in the hepatocytes were also confirmed by immunostaining. As a result, as shown in FIG. 8C, the expressions of SOCS1 and SOCS3 proteins were increased in the hepatocytes over-expressing TM4SF5 protein compared to the control group (FIG. 8C).

Meanwhile, the hepatocytes over-expressing SREBP1 protein isolated from the normal mouse, and the expression changes of SOCS1 and SOCS3 proteins were confirmed by Western blotting using the prepared hepatocytes. As a result, as shown in FIG. 8D, the expressions of SOCS1 and SOCS3 proteins were increased in the hepatocytes over-expressing SREBP1 protein compared to the control group (FIG. 8D).

The primary hepatocytes isolated from the normal mouse (52 weeks old) were transfected with SOCS3 (NM_174466) shRNA (shSOCS3, sense 5′ CAACAUCUCUGUCGGAAGAUU-3′ SEQ. ID. NO: 111; antisense 5′ UCUUCCGACAGAGAUGUUGUU-3′ SEQ. ID. NO: 112) under the same conditions and methods as described in Example <4-1> to prepare hepatocytes wherein the expression of SOCS3 gene was suppressed, and the expression changes of SREBP1 and SOCS3 proteins and the phosphorylation changes of STAT3 were confirmed by Western blotting.

As a result, as shown in FIG. 8E, the expression of SOCS3 gene was decreased in the hepatocytes compared to the control group (FIG. 8E).

### Example 7: Preparation of TM4SF5 Gene Knock-Out (KO) Mouse

### <7-1> Preparation of TM4SF5 Gene KO Mouse

First, the cas9/RGEN KO mouse in which exon 3 of the Tm4sf5 mouse gene (GenBank accession number: NM_029360.3) composed of 5 exons was removed was prepared using C57BL/6 mouse (Macrogen, Seoul). At this time, the mouse in which 522 bp of DNA containing TM4SF5 gene was deleted was obtained using the RGEN site shown in Table 7. In addition, the mouse in which TM4SF5 gene was deleted was prepared from the mouse obtained above using the mouse TM4SF5 primers shown in Table 7 below.

TABLE 7

| Name | Sequence (5′→3′) | SEQ. ID. NO: |
|---|---|---|
| RG1 | GCGGGAGCTGGGCTCCGAATTGG | SEQ. ID. NO: 70 |
| RG2 | TTAAGCATTTGGGTCCAATTCGG | SEQ. ID. NO: 71 |
| RG3 | TGAGAAATCCTGTTTGATCTTGG | SEQ. ID. NO: 72 |
| RG4 | AGGTATTAGGGGTGGCCTATGGG | SEQ. ID. NO: 73 |
| mouse | GTAGTATGCGGGAGGCACTG | SEQ. ID. NO: 74 |
| TM4SF5_ | | |
| forward | | |
| mouse | GGGTGACCACTCAGACTTCC | SEQ. ID. NO: 75 |
| TM4SF5_ | | |
| reverse | | |

The mutant mouse was selected by observing the heterologous double-strand formation between the wild-type (normal type) and mutant PCR products through T7E1 analysis.

In addition, using C57BL/6 mouse, the cas9/RGEN KO mouse in which exon 1 of the Tm4sf5 mouse gene (GenBank accession number: NM_029360.3) was removed was prepared. At this time, the mouse in which 29 bp of DNA containing TM4SF5 gene was deleted was obtained using the RGEN site shown in Table 8. In addition, the Tm4sf5-Exon 1-KO mouse in which TM4SF5 gene was deleted was prepared from the mouse obtained above using the mouse TM4SF5 primers shown in Table 7 below. In other examples except FIG. 9, the Tm4sf5-Exon 1-KO mouse was used as the Tm4sf5-KO mouse.

TABLE 8

| Name | Sequence (5′→3′) | SEQ. ID. NO: |
|---|---|---|
| RG1 | GAGGTTGCCGTCCGTCCAGGTGG | SEQ. ID. NO: 107 |
| RG2 | GCTGAGGTTGCCGTCCGTCCAGG | SEQ. ID. NO: 108 |
| mouse | ACTTCCTCAGGGCCTCTCTC | SEQ. ID. NO: 109 |
| TM4SF5_ | | |
| forward | | |
| mouse | CCTTTCCCACATTCCTCAGA | SEQ. ID. NO: 110 |
| TM4SF5_ | | |
| reverse | | |

The mutant mouse was selected by observing the heterologous double-strand formation between the wild-type (normal type) and mutant PCR products through T7E1 analysis.

<7-2> Confirmation of Expression Changes of Factors that Regulate Phosphorylation of STAT3 Protein in TM4SF5 Gene KO Mouse

The expression changes of SOCS1 and SOCS3 genes that regulate the phosphorylation of STAT3 protein in the TM4SF5 gene KO mouse prepared in Example <7-1> were confirmed. The expression changes of SOCS1 and SOCS3 genes were confirmed under the same conditions and methods as described in Example <2-1> using the hepatocytes obtained from the mouse prepared above, except that the primers listed in Table 3 were used. As a result, as shown in FIG. 12A, the expressions of SOCS1 and SOCS3 genes were suppressed by KO of TM4SF5 gene (FIG. 12A).

In addition, Western blotting was performed to confirm whether the suppression of the expressions of SOCS1 and SOCS3 genes was the same in proteins. The experiment was performed under the same conditions and methods as described in Example <2-2>, except that the hepatocytes obtained from the mouse prepared above were used and SOCS1, SOCS3 and β-tubulin were used as the primary antibodies. As a result, as shown in FIG. 12B, the expressions of SOCS1 and SOCS3 proteins in cells of the TM4SF5 gene KO mouse were also suppressed (FIG. 12B).

### Example 8: Inhibition of Fat Accumulation in TM4SF5 Gene KO Mouse Fed High-Fat Diet

### <8-1> Inhibition of Fat Accumulation in TM4SF5 Gene KO Mouse Fed High-Fat Diet

Fat accumulation in the liver of the TM4SF5 gene KO mouse fed a high-fat diet was confirmed by H&E staining.

First, the TM4SF5 gene KO mouse prepared in Example <7-1> was fed a 60% kcal high fat diet (Harlan, USA) for 10 weeks. The weight changes were measured weekly during the 10 weeks. Ten weeks later, H&E staining was performed under the same conditions and methods as described in Example <1-3>, except that the liver tissue was obtained from the mouse.

As a result, as shown in FIGS. 10A, 10B and 12C, the fat accumulation was inhibited in the TM4SF5 gene KO mouse compared to the normal mouse despite the high fat diet (FIGS. 10A, 10B and 12C).

### <8-2> Confirmation of Expression Changes of Fat-Related Genes and Proteins in TM4SF5 Gene KO Mouse Fed High-Fat Diet

The liver tissue was obtained from the TM4SF5 gene KO mouse fed a high fat diet, and the expression changes of the fat-related genes and proteins in the liver tissue were confirmed.

The experiment was performed to confirm the expression changes of the genes under the same conditions and methods as described in Example <2-1> using the hepatocytes obtained from the mouse prepared above, except that the primers listed in Table 9 below were used.

TABLE 9

| Name | Sequence (5′→3′) | SEQ. ID. NO: |
|---|---|---|
| Fasn_F | TCTGGGCCAACCTCATTGGT | SEQ. ID. NO: 76 |
| Fasn_R | GAAGCTGGGGGTCCATTGTG | SEQ. ID. NO: 77 |
| Pparγ_F | CTGGCCTCCCTGATGAATAAAG | SEQ. ID. NO: 78 |
| Pparγ_R | AGGCTCCATAAAGTCACCAAAG | SEQ. ID. NO: 79 |
| L-Fabp_F | TGGACCCAAAGTGGTCCGCA | SEQ. ID. NO: 80 |
| L-Fabp_R | AGTTCAGTCACGGACTTTAT | SEQ. ID. NO: 81 |
| Srebf-1c_F | GTGTTGGCCTGCTTGGCTCT | SEQ. ID. NO: 82 |
| Srebf-1c_R | GAGCAGCCTGGGGGAAATCT | SEQ. ID. NO: 83 |
| β-actin_F | GGCCGGGACCTGACAGACTA | SEQ. ID. NO: 84 |
| β-actin_R | AGGAAGAGGATGCGGCAGTG | SEQ. ID. NO: 85 |

On the other hand, Western blotting was performed under the same conditions and methods as described in Example <2-2>, except that antibodies against SREBP1 precursor, mature SREBP1, CD36 (Santa cruz, USA) and α-tubulin (Cell Signaling Technology, USA) were used as the primary antibodies.

As a result, as shown in FIGS. 11 and 12D, the expressions of the fat-related Srebp1, Srebp1c, Srebp2, Ldlr, ApoB100, CD36, Fasn and Pparγ genes and proteins were suppressed by KO of TM4SF5 gene compared to the normal control group (FIGS. 11 and 12D).

### <8-3> Confirmation of Fat Level Changes in Liver Tissue of TM4SF5 Gene KO Mouse Fed High-Fat Diet

For the measurement of fat in the liver tissue of the TM4SF5 gene KO mouse fed a high-fat diet, the tissue fixed to RNAlater was cut into pieces of ˜10 mg, and cholesterol (Abcam, ab65390), free fatty acid (Abcam, ab65341) and Triglyceride (Cell biolabs, STA-396) were measured.

As a result, as shown in FIGS. 10C and 10D, the levels of cholesterol and FFA in the liver tissue were increased in the normal mouse fed a high fat diet, but the levels of cholesterol and FFA were not increased in the liver tissue of the TM4SF5 gene KO mouse fed a high-fat diet (FIGS. 10C and 10D).

### Example 10: Confirmation of Interaction of TM4SF5 and APC Genes

### <10-1> Confirmation of Characteristics of Offspring Obtained by Mating TM4SF5 Gene KO Mouse and APC^{min/+} Mouse

The phenotype of the offspring obtained by mating the TM4SF5 gene KO mouse prepared in Example <7-1> with the APC^{min/+} mouse mutated to develop colon disease more likely (Central Lab. Animal Inc., Seoul, Korea) was confirmed.

The expressions of TM4SF5 and APC genes were confirmed under the same conditions and methods as described in Example <2-1> using the liver tissue of the obtained offspring, except that the primers listed in Table 10 below were used.

TABLE 10

| Name | Sequence (5′→3′) | SEQ. ID. NO: |
|---|---|---|
| Tm4sf5_F | GTCTTCTCCTCCGCCTTTG | SEQ. ID. NO: 86 |
| Tm4sf5_R | GGTAGTCCCACTTGTTGTCTATT | SEQ. ID. NO: 87 |
| MAPC MT | TGAGAAAGACAGAAGTA | SEQ. ID. NO: 88 |
| MAPC 15 | TTCCACTTTGGCATAAGGC | SEQ. ID. NO: 89 |
| MAPC 9 | GCCATCCCTTCACGTTAG | SEQ. ID. NO: 90 |
| β-actin_F | GGCCGGGACCTGACAGACTA | SEQ. ID. NO: 91 |
| β-actin_R | AGGAAGAGGATGCGGCAGTG | SEQ. ID. NO: 92 |

In addition, the obtained offspring were sacrificed and each organ was observed. The results are shown in FIG. 13B. As shown in FIG. 13B, the offspring exhibited the symptoms of portal hypertension such as enlarged spleen and open sinusoid in addition to splenomegaly and abnormal intestines, which are the characteristics typically observed in APC^{+/-} mouse (FIG. 13B).

### <10-2> Confirmation of Expressions of Fat and Collagen in Offspring Obtained by Mating TM4SF5 Gene KO Mouse and APC^{min/+} Mouse

H&E and Mason's trichrome stainings were performed using the liver tissue of the obtained offspring. At this time, H&E staining was performed as described in Example <1-3>.

On the other hand, for Mason's trichrome staining, the liver tissue fixed to paraffin was left in a 60° C. oven for about 20 minutes to remove paraffin. The paraffin-removed tissue was placed in a heated Bouin's solution, followed by reaction for 1 hour. Upon completion of the reaction, the liver tissue was washed with tap water, placed in a hematoxylin solution, and reacted for 10 minutes. The liver tissue was washed again with tap water, placed in a biebrich scarlet-acid fushsin solution, and reacted for 5 minutes. Upon completion of the reaction, the liver tissue was placed in distilled water, and then placed in a phosphotungstic acid/phosphomolybdic acid solution, followed by reaction for 15 minutes. Thereafter, the liver tissue was reacted in an aniline blue solution for 10 minutes and 1% acetic acid for 1 minute, respectively, and then the tissue was dehydrated. The dehydrated tissue was placed in xylene, taken out, placed on a slide and mounted. The cells stained with the said two staining methods were observed using a microscope, and the results are shown in FIG. 13D.

As shown in FIG. 13D, the cell arrangement was abnormally smooth around the region showing the symptoms of portal hypertension in the liver tissue of the offspring obtained by mating TM4SF5 gene KO mouse and APC^{min/+} mouse, and the expression of collagen was increased (FIG. 13D).

### <10-3> Confirmation of Expression of TM4SF5 in Hepatocytes of Offspring Obtained by Mating TM4SF5 Gene KO Mouse and APC^{min/+} Mouse

Immunostaining was performed to confirm the expression changes of TM4SF5, β-catenin and HIF1α proteins in the offspring obtained in Example <10-1>. The experiment was performed under the same conditions and methods as described in Example <2-3>, except that the antibodies against TM4SF5, β-catenin and HIF1α proteins were used as the primary antibodies.

As a result, as shown in FIG. 13C, the expressions of TM4SF5, β-catenin and HIF1α proteins were increased and blood vessels were expanded in the hepatocytes of the offspring obtained by mating TM4SF5 gene KO mouse and APC^{min/+} mouse (FIG. 13C). Therefore, it was confirmed that portal hypertension, a vasodilation symptom of the liver tissue, was related to the expression of TM4SF5, and this portal hypertension was related to liver fibrosis and liver cirrhosis (Methods Mol Biol. 2017; 1627: 91-116).

### <10-4> Confirmation of Fat-Related Signal Transduction Mechanism in Hepatocytes of Offspring Obtained by Mating TM4SF5 Gene KO Mouse and APC^{min/+} Mouse

The fat-related signal transduction mechanism was confirmed in the hepatocytes of the offspring obtained by mating TM4SF5 gene KO mouse and APC^{min/+} mouse by Western blotting. The experiment was performed under the same conditions and methods as described in Example <2-3>, except that the hepatocytes of the offspring obtained in Example <10-1> were used and the antibodies against laminin, fibronectin, pY142 β-catenin, pY705 STAT3, STAT3, pS9-GSK3β, GSK3β and TM4SF5 proteins were used as the primary antibodies.

As a result, as shown in FIG. 13E, The expressions of laminin and fibronectin proteins and the phosphorylation of GSK3β were increased in the hepatocytes of the offspring obtained by mating TM4SF5 gene KO mouse and APC^{min/+} mouse (FIG. 13E).

Therefore, it was confirmed that the expression of TM4SF5 protein caused disorders in the blood vessels and the portal vein of the liver, and induced fibrosis symptoms in the liver by promoting the expression of the fibrosis-related extracellular matrix.

### Example 11: Confirmation of Extracellular Arginine Transport by TM4SF5 Protein

It was confirmed that the characteristics of fatty liver were appeared by the over-expression of TM4SF5 protein. Immunoprecipitation was performed to confirm whether the TM4SF5 protein was bound to SLC7A1 or SLC38A9, the mTOR and arginine transporter.

First, HEK293T cells (KCLB, Korea) were prepared by culturing in 5% CO₂ at 37° C. using DMEM containing 10% FBS and antibiotics. The prepared cells were distributed in 100 mm plates and cultured to the density of 60%, which were transfected with the construct expressing SLC7A1 or SLC38A9 protein labeled with HA tag or the construct labeled with STERP tag. The cells cultured for 2 days after the transfection were washed once with PBS and incubated in 5% CO₂ at 37° C. for 50 minutes in the amino acid- or arginine-free medium. After the incubation, the cells were washed twice with PBS, and 500 μl of lysis buffer was added thereto, followed by reaction at 4° C. for 15 minutes. The cell lysate was centrifuged for 15 minutes at 4° C., 12,000×g to obtain supernatant. The protein included in the supernatant was quantified using BCA reagent (Thermo Scientifics, USA), and the beads coated with streptavidin were added thereto in proportion to the protein amount. The mixture was reacted at 4° C. for 4 hours while rotating, and then centrifuged at 4° C., 7,000×g for 5 minutes. After the centrifugation, lysis buffer was added to the obtained pellets, which were lightly mixed and then centrifuged for 5 minutes at 4° C., 7,000×g to obtain pellets. This washing process was repeated twice using lysis buffer and twice using PBS, and then 2× sample buffer was added to the washed pellets, which were boiled for 5 minutes to prepare a sample. Western blotting was performed under the same conditions and methods as described in Example <2-3>, except that the prepared sample was used, and HA (Covanvce, USA) and streptavidin-HRP (IBA, USA) were used as the primary antibodies.

As a result, as shown in FIG. 14, TM4SF5 protein was bound to mTOR, SLC7A1 or SLC38A9, and this binding was stronger in a situation where arginine was deficient in the culture medium of the cells (FIGS. 14A, 14B and 14C). It was also confirmed that the phosphorylation levels of S6K, 4EBP1 and ULK1 were increased as the amino acid was depleted and repleted to the cells when TM4SF5 protein was expressed compared to when TM4SF5 protein was not expressed (FIGS. 14D and 14E).

### Example 12: Confirmation of Correlation of TM4SF5 Protein and Arginine Transport Mechanism

### <12-1> Confirmation of Arginase in TM4SF5 Gene KO Mouse

The TM4SF5 gene KO mouse was starved for 6 hours, and the content of arginase present in the liver was confirmed by measuring the expression of arginase 1 gene.

Particularly, the TM4SF5 gene KO mouse prepared in Example <7-1> was starved for 6 weeks, and then sacrificed to obtain the liver tissue. Real-time PCR was performed under the same conditions and methods as described in Example <2-1>, except that the obtained liver tissue was used and the primers known for the arginase gene were used.

As shown in FIG. 15A, the expression of arginase 1, the arginine degrading enzyme gene, was significantly decreased in the TM4SF5 gene KO mouse group starved for 16 hours (white bar, FIG. 15A, black bar=16 hours of starvation and 4 hours of intake).

### <12-2> Confirmation of Binding of TM4SF5 Protein and Arginine

The following experiment was performed to confirm whether TM4SF5 protein directly affects the transport of arginine.

First, HEK293FT cells (Thermo, USA) were prepared by culturing in 5% CO₂ at 37° C. using DMEM containing 10% FBS and antibiotics. The prepared cells were distributed in 150 mm plates and cultured to the density of 60%, which were transfected with the construct expressing TM4SF5, MetaP2, Castrol, TM4SF1, TM4SF4 and TM4SF5 proteins constructed in Example 11 using PEI. Two days after the transfection, the desired protein was precipitated under the same conditions and methods as described in Example 11 using the beads coated with streptavidin. The precipitate was added with 10 μM [3H]-arginine (American radiolabeled chemicals, USA), followed by reaction at 4° C. for 1 hour. At this time, a sample in which 10 mM of L-arginine was added to the same amount of beads was used as the control. Upon completion of the reaction, the beads were washed 3 times with lysis buffer, to which 2 m [symbol] of scintillation cocktail (Ultima gold, Perkin Elmer, USA) was added. The mixture was vortexed and analyzed using a liquid scintillation counter (Tri-Carb, Perkin Elmer, USA).

As a result, as shown in FIGS. 15B and 15C), TM4SF5 protein and Castor1 protein, known as the arginine sensor present in the cytoplasm, were directly bound to arginine (FIGS. 15B and 15C).

### <12-3> Confirmation of Concentration-Dependent Binding of TM4SF5 Protein and Arginine

An experiment was performed to confirm whether the binding of TM4SF5 protein and arginine confirmed in Example <12-2> was concentration-dependent. The experiment was performed under the same conditions and methods as described in Example <12-2>, except that HEK293FT cells transformed with TM4SF5 protein were used and 0, 0.01, 0.05. 0.1 or 0.5 mM L-arginine was added.

As a result, as shown in FIGS. 15D and 15E, TM4SF5 protein was bound to arginine concentration-dependently (FIGS. 15D and 15E).

### Example 13: Confirmation of Binding Position of TM4SF5 Protein and Arginine

It was confirmed that TM4SF5 protein was directly bound to arginine, so the following experiment was performed to confirm which residue of TM4SF5 protein played an important role in binding to arginine.

First, a short extracellular loop (SEL) fragment mutant comprising the 31^{st} to 42^{nd} amino acid residues from the N-terminus of the amino acid sequence constituting TM4SF5 protein (SEQ. ID. NO: 1) and a long extracellular loop (LEL) fragment mutant comprising the 113^{th} to 157^{th} amino acid residues from the N-terminus were prepared. Or mutants of TM4SF5 protein were prepared by substituting the 124^{th} to 129^{th} amino acid residues and the 153^{th} to 157^{th} amino acid residues from the N-terminus, respectively, were prepared. As a result, SEL, LEL, W124A, G125A, Y126S, H127A, F128S, E129A, P153A, W154A, N155Q, V156A or T157A mutant was obtained in addition to the wild type of TM4SF5 protein (WT, full length). The binding of TM4SF5 protein and arginine was confirmed under the same conditions and methods as described in Example <12-2>, except that the construct expressing the obtained mutant protein was used.

As a result, as shown in FIG. 15F, the short extracellular loop (SEL) mutant of TM4SF5 protein was not bound to arginine (FIG. 15F). Therefore, it was confirmed that LEL amino acid residues of TM4SF5 were bound to arginine.

As shown in FIG. 15G, the mutant in which the 124^{th} to 129^{th} amino acid residues present in the extracellular loop of TM4SF5 protein was not bound to bind arginine (FIG. 15G). Therefore, it was 15G). Therefore, it was confirmed that the 124^{th} to 129^{th} amino acid residues from the N-terminus of TM4SF5 protein were bound to arginine.

On the other hand, as shown in FIG. 15G, the above region is a site known to form cation-n interaction, and is a conserved sequence in most animal TM4SF5 proteins (FIG. 15G).

### Example 14: Confirmation of Body Weight Change in TM4SF5 Gene KO Mouse by High Arginine Diet

### <14-1> Confirmation of Body Weight Change in TM4SF5 Gene KO Mouse by High Arginine Diet

The body weight changes in the TM4SF5 gene KO mouse by the high arginine diet (High Arg Diet) were confirmed by the following method.

Particularly, the TM4SF5 gene KO mouse prepared in Example <7-1> was fed L-arginine (40 g/kg of mouse body weight) for 10 weeks. The weight changes were measured weekly during the 10 weeks, and the results are shown in FIG. 17A.

As shown in FIG. 17A, the normal mouse fed a high arginine diet gained about 25% in weight compared to the mouse fed a normal diet, whereas the TM4SF5 gene KO mouse gained about 7% in weight (FIG. 17A). On the other hand, as shown in FIG. 17B, as a result of confirming the weight gain of each individual mouse in comparison to the starting point of the high arginine diet, the weight gain of the TM4SF5 gene KO mouse was significantly decreased (FIG. 17B).

### <14-2> Confirmation of Fat Accumulation in TM4SF5 Gene KO Mouse by High Arginine Diet

The liver tissue was extracted from the TM4SF5 gene KO mouse fed a high arginine diet in Example <13-1>, and H&E staining was performed using the method described above.

As a result, as shown in FIG. 17C, fatty liver was induced in the normal mouse fed a high arginine diet, whereas the fat accumulation was relatively suppressed in the liver tissue of the TM4SF5 gene KO mouse (FIG. 17C).

### Example 15: Confirmation of Relationship Between TM4SF5 Protein and Glucose Transporter

### <15-1> Confirmation of S6K Phosphorylation by TM4SF5 Protein

The proteins binding to TM4SF5 protein were analyzed by mass spectrometry, and GLUT1 (SLC2A1) protein was selected. The said GLUT1 protein is a glucose transporter, which is involved in the production of energy by moving into the cell membrane by insulin and supplying glucose inside the cell. Thus, the phosphorylation of S6Kinase was confirmed as follows using the cells transformed with a construct expressing TM4SF5 protein.

First, HEK293FT cells (Thermo, USA) were prepared by culturing in 5% CO₂ at 37° C. using DMEM containing 10% FBS and antibiotics. The survival responsiveness of the cells was confirmed by investigating the survival of the prepared cells under the stress such as re-supply after deficiency of glucose.

As a result, as shown in FIG. 18A, the phosphorylation of S6K was increased in the cell line expressing TM4SF5 protein (FIG. 18A).

### <15-2> Measurement of Glycolytic Stress According to Inhibition of TM4SF5 Protein Expression

Glycolytic stress in the cells in which the expression of TM4SF5 protein was suppressed was measured using an XF analyzer (Sea Horse). To construct a TM4SF5 expression-suppressing cell line, the HEK293FT cell line was transfected with the pLKO.1 (addgene) lenti-viral plasmid, psPAX2 and pDM2.G constructs in which shRNA sequences (shTM4SF5 #2: 5′-accauguguacgggaaaaugugc-3′, SEQ. ID. NO: 95; shTM4SF5 #4, 5′-ccaucucagcuugcaaguc-3′, SEQ. ID. NO: 96) targeting TM4SF5 was inserted using PEI. After 5 hours, the culture medium was replaced and cultured for 24 hours to obtain shTM4SF5 lenti-virus. Hep3B cells were infected with the obtained virus and 4 ug/ml of polybrene for 24 hours, followed by selection with puromycin for 48 hours.

Particularly, Hep3B cells were distributed in XFp cell culture plates (Sea Horse bioscience, USA) at the density of 5×10³ cells/well. The cells were cultured in 5% CO₂ at 37° C. for 16 hours and then the medium was replaced with Sea Horse XF basal medium (Sea Horse bioscience, USA). The cells in the replaced medium were cultured for 1 hour in a 37° C. incubator without CO₂ supply. The XFp cell culture plate containing the cultured cells was bound to a hydrated and calibrated sensor cartridge (Sea Horse bioscience, USA) at 37° C. and analyzed using an XFp analyzer. 100 mM glucose (A), 50 μM oligomycin (B), and 500 mM 2-deoxy-D-glucose (C) were loaded through the drug inlet.

As a result, as shown in FIG. 18B, the reactivity to glucose was reduced by suppressing the expression of TM4SF5 gene (FIG. 18B).

### <15-3> Confirmation of Expression Changes of Glycolysis Related Genes by Over-Expression of TM4SF5 Protein

The following experiment was performed to confirm the expression changes of the glycolysis-related genes in the cells over-expressing TM4SF5 protein.

First, a SNU449 liver cancer cell line was transformed with a construct expressing TM4SF5 protein. The cells were crushed by adding liquid nitrogen, and RNA was extracted using an RNAeasy kit (Qiagen, USA) according to the manufacturer's protocol. DNAse was added to the extracted RNA to remove DNA, and cDNA was synthesized by the conventional method. An adapter was attached to the synthesized cDNA, which was amplified by PCR, and the PCR products having the size of 200 to 400 bp were selected. The sequence of the selected cDNA was analyzed using a HiSeq 4000 sequencer (Illumina, USA). Artifacts were removed through pre-processing of the sequencing results and mapped to the genome using a HISTA2 program. The expression levels were obtained through transcript assembly using StringTie from the mapped data.

As a result, as shown in FIG. 18C, the expressions of the glycolysis-related genes present in the cells were increased by the over-expression of TM4SF5 protein (FIG. 18C).

### Example 16: Confirmation of Effect of High-Carbohydrate Diet or High-Sucrose Diet in TM4SF5 Gene KO Mouse

### <16-1> Confirmation of Suppression of Weight Gain by High-Carbohydrate Diet or High-Sucrose Diet in TM4SF5 Gene KO Mouse

The weight changes of the TM4SF5 gene KO mouse fed a high-carbohydrate diet (70% kCal high-carbohydrate) or a high-sucrose diet (AIN-93G diet; It has a sucrose content of 10%, which is 3 times higher than that of a chow diet with a sucrose content of 3.15%.) under the same conditions and methods as described in Example <8-1> were confirmed, and the results are shown in FIGS. 16A and 19A, respectively.

As shown in FIGS. 16A, 16B and 19A, the weight of the normal mouse was significantly increased in the case of the high-carbohydrate diet compared to the case of the normal diet, but the weight of the TM4SF5 gene KO mouse was not significantly increased (FIGS. 16A and 16B). On the other hand, the normal mouse fed a high-sucrose diet showed a high rate of weight gain, but the rate of weight gain of the TM4SF5 gene KO mouse was not high (FIG. 19A).

### <16-2> Confirmation of Glucose Resistance Changes by High-Carbohydrate Diet or High-Sucrose Diet in TM4SF5 Gene KO Mouse

The glucose resistance of the TM4SF5 gene KO mouse fed a high-carbohydrate diet or a high-sucrose diet under the same conditions and methods as described in Example <8-1> was measured by the following method.

Particularly, the mice fed a high-carbohydrate diet or a high-sucrose diet for 3 weeks and 10 weeks were starved for 16 hours, and blood was collected from the tail. Blood glucose in the collected blood was measured using a blood glucose meter (One touch ultra, Johnsons and Johnsons, USA). After measuring the blood glucose, 2 g/kg of glucose was injected into the mouse intraperitoneally, and blood was collected from the tail 30 minutes, 60 minutes, 90 minutes and 120 minutes after the injection. Then, blood glucose was measured.

As a result, as shown in FIGS. 16C and 19B, the glucose resistance of the TM4SF5 gene KO mouse was reduced by the high-carbohydrate diet or high-sucrose diet for 10 weeks compared to that of the normal mouse (FIGS. 16C and 19B).

### <16-3> Confirmation of Effect of High-Carbohydrate Diet or High-Sucrose Diet on Insulin Resistance of TM4SF5 Gene KO Mouse

The insulin resistance of the TM4SF5 gene KO mouse fed a high-carbohydrate diet or a high-sucrose diet under the same conditions and methods as described in Example <8-1> was measured by the following method.

Particularly, the mice fed a high-carbohydrate diet or a high-sucrose diet for 10 weeks were starved for 6 hours, and blood was collected from the tail. Blood glucose in the collected blood was measured using a blood glucose meter (One touch ultra, Johnsons and Johnsons, USA). After measuring the blood glucose, 0.5 U/kg of insulin was injected into the mouse intraperitoneally, and blood was collected from the tail 30 minutes, 60 minutes, 90 minutes and 120 minutes after the injection. Then, blood glucose was measured.

As a result, as shown in FIGS. 16D and 19B, the insulin resistance was not related to the presence of TM4SF5 protein, unlike the glucose resistance (FIG. 16D), but the insulin resistance of the TM4SF5 gene KO mouse was reduced by the high-sucrose diet for 10 weeks (FIG. 19B).

### <16-4> Confirmation of Effect of High-Carbohydrate Diet or High-Sucrose Diet on Blood AST, ALT, Triglyceride and Cholesterol Levels in TM4SF5 Gene KO Mouse

The levels of blood AST, ALT and cholesterol in the TM4SF5 gene KO mouse fed a high-carbohydrate diet or a high-sucrose diet under the same conditions and methods as described in Example <8-1> was measured using Fuji Dri-Chem 3500i.

As a result, as shown in FIGS. 16E and 19C, the levels of blood ALT, AST, total cholesterol and triglyceride in the normal mouse fed the high-carbohydrate diet were increased, but the levels in the TM4SF5 gene KO mouse fed the high-carbohydrate diet were not much increased (FIG. 16E). On the other hand, the levels of blood ALT and AST in the normal mouse fed the high-sucrose diet were increased, but the levels in the TM4SF5 gene KO mouse were not much increased, and the levels of total cholesterol and triglyceride were not changed without statistical significance (FIG. 19C).

### <16-5> Confirmation of Fat Accumulation in TM4SF5 Gene KO Mouse by High-Carbohydrate Diet or High-Sucrose Diet

H&E staining was performed using the liver tissue of the TM4SF5 gene KO mouse fed a high-carbohydrate diet or high-sucrose diet in Example <16-1> using the method described above.

As a result, as shown in FIG. 19D, fatty liver was induced in the normal mouse fed the high-carbohydrate or high-sucrose diet, whereas the fat accumulation was relatively suppressed in the liver tissue of the TM4SF5 gene KO mouse fed the high-carbohydrate or high-sucrose diet (FIG. 19D).

### <16-6> Confirmation of Accumulation of Monoacyl-, Diacyl- and Triacyl-Glycerol Synthesis in TM4SF5 Gene KO Mouse by High-Carbohydrate Diet or High-Sucrose Diet

The liver tissue was extracted from the TM4SF5 gene KO mouse fed a high-carbohydrate diet or high-sucrose diet in Example <16-1>. After lysophilization of the liver tissue and pulverizing thereof using a mortar, lipids were extracted with 0.3 ml of methanol and 0.1% butylated hydroxytoluene solution per 10 mg of the liver tissue. After adding methyl-tert-butyl ether containing 0.1% butylated hydroxytoluene to the extract, the mixture was shaken for 1 hour at room temperature. The mixture was diluted with 0.25 ml of H2O, vortexed at room temperature for 10 minutes, and centrifuged at 14,000 g at 4° C. for 15 minutes. The supernatant and the lower solution were separated, followed by drying. After treating 40 μl of CHCl₃:MeOH (1:9) to 0.16 ml of the solution, lipid analysis was performed using LC-MS/MS (8040, Shimadzu, Japan).

As a result, as shown in FIG. 19E, the synthesis of monoacyl-, diacyl- and triacyl-glycerol in the liver tissue of the TM4SF5 gene KO mouse fed the high-sucrose diet was low compared to the normal mouse fed the high-sucrose diet (FIG. 19E).

### Example 17: Confirmation of Liver Cirrhosis Symptoms Induced by Over-Expression of TM4SF5 Protein

The mice over-expressing TM4SF5 protein were prepared under the same conditions and methods as described in Example <1-1>, which were bred for 78 weeks. The mice were sacrificed as described above, and the liver tissue was obtained therefrom. The phenotype of the liver tissue was confirmed by H&E and Mason's trichrome staining. As a result, as shown in FIG. 20A, the liver tissue showed the phenotype of liver cirrhosis with fibrosis (FIG. 20A). Since the mice were old (78 weeks (1 year and 6 months) old), the symptoms of mild fatty liver were shown even in the normal mouse, but the symptoms of severe fatty liver and extramedullary hemorrhage were observed in the animal over-expressing TM4SF5 (FIG. 20B).

In addition, the expression changes of the fat-related proteins were confirmed by Western blotting as described above using the liver tissue, and the results are shown in FIG. 20C.

As shown in FIG. 20C, the phosphorylation of STAT3 was increased and the level of extracellular matrix (ECM), a major factor of liver cirrhosis, was increased in the 78-week-old mouse by the over-expression of TM4SF5 protein, unlike the 52-week-old mouse showing the phenotype of fatty liver. On the other hand, the expression of SREBP1 protein was suppressed, and the expression of SIRT1 protein was increased, thereby the fat synthesis and accumulation in the liver tissue were reduced (FIG. 20C).

In addition, immunostaining was performed as described above using the liver tissue, and the results are shown in FIG. 21A. As shown in FIG. 21A, the expressions of SOCS1 and SOCS3 proteins were suppressed, the phosphorylation of STAT3 was increased, and the expressions of ECMs such as α-SMA, collagen 1 and laminin were increase by the over-expression of TM4SF5. At this time, collagen 1 and α-SMA showed similar expression patterns, whereas laminin and laminin γ2 showed different expression cells and expression patterns (FIG. 21A).

On the other hand, the expression changes of the genes related to fat metabolism, liver cirrhosis and hepatitis were confirmed using the liver tissue as described above. As a result, as shown in FIGS. 21B and 21C, the expressions of the genes related to fat metabolism were not affected by the over-expression of TM4SF5 protein, but the expressions of the genes related to liver cirrhosis and hepatitis were increased (FIGS. 21B and 21C).

Therefore, it was confirmed that fatty liver developed in liver cirrhosis or hepatitis after a certain period of time in the transgenic mouse over-expressing TM4SF5 protein, thereby the phosphorylation of STAT3 protein or the ECM level was increased.

### Example 18: Confirmation of Expression Changes of TM4SF5 Protein in Liver Disease Mouse Model

It was confirmed that the fatty liver produced in the mouse over-expressing TM4SF5 protein showed the symptoms of liver cirrhosis and hepatitis over time. In general, it has been reported that the mouse administered with carbon tetrachloride for 4 weeks showed the symptoms of liver fibrosis, and the mouse administered for 16 weeks shows the symptoms of cirrhosis. Thus, the expression changes of TM4SF5 protein were confirmed in the model mouse induced with liver cirrhosis by a drug.

First, a mouse model in which liver disease was induced by intraperitoneally injecting carbon tetrachloride (CCl₄) once a week for 1, 4 or 16 weeks at the concentration of 1 mg/kg to 4-week-old BALB/C mice (Orient Bio, Korea) was prepared. H&E and Mason's trichrome stainings were performed using the prepared model mouse, and the results are shown in FIG. 22A.

As shown in FIG. 22A, the cells in the liver tissue of the mouse administered with CCl⁴ for 4 or 16 weeks died centering on the blood vessels, and an immune response occurred around the cells, and the cells with altered morphology were observed compared to the normal cells. In addition, as collagen accumulated between the cells, a path was generated between the blood vessels (FIG. 22A).

The expression levels of proteins and mRNAs were confirmed as described above using the liver tissue of the model mouse, and the results are shown in FIG. 23. As shown in FIG. 23A, the expression of TM4SF5 protein, the phosphorylation of STAT3 protein and the level of ECM were all increased in the liver tissue of the model mouse (FIG. 23A). In addition, it was confirmed that the mRNA levels of elastin, laminin α2, α3, α5, γ2, and γ3 chains in the liver tissue with liver cirrhosis of the animal administered with CCl₄ for 4 or 16 weeks were higher than those of the control group not treated with CCl₄ (FIG. 23B).

In addition, immunostaining was performed as described above using the liver tissue of the model mouse, and the results are shown in FIG. 24. As shown in FIG. 24, the phosphorylation of STAT3 was increased, and the expressions of α-SMA, collagen I, collagen IV, laminin and laminin γ2 proteins were increased as the expression of TM4SF5 protein increased in the liver tissue of the model mouse (FIG. 24).

On the other hand, after administering CCl₄ to the TM4SF5 gene KO mouse prepared in Example <7-1> as described above, the liver tissue was obtained and Mason's trichrome staining was performed, and the results are shown in FIG. 22C. As a result, it was confirmed that the accumulation of collagen was decreased in the TM4SF5 gene KO mouse compared to the control group (FIG. 22C).

### Example 19: Confirmation of Control Mechanism of Laminin Protein Expression in Liver Disease Model Mouse

The mechanism of regulating the expression of laminin protein was confirmed by the following method using the liver tissue of the liver disease model mouse prepared by drug administration in Example 18.

First, hepatocytes were obtained from the isolated liver tissue as described above. The expressions of TM4SF5 and STAT3 proteins in the obtained hepatocytes were suppressed by transfecting the cells with shTM4SF5 or silencing STAT3 [On-Target plus SMART pool siRNA (Thermo)], and then the expression changes of laminin were confirmed by Western blotting as described above.

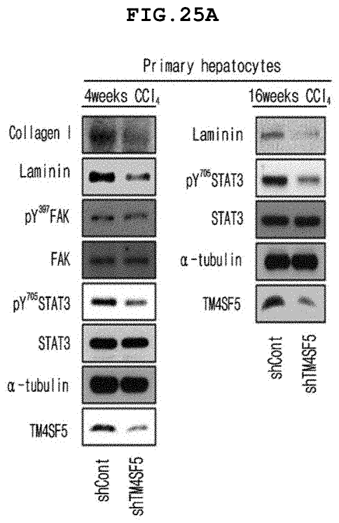

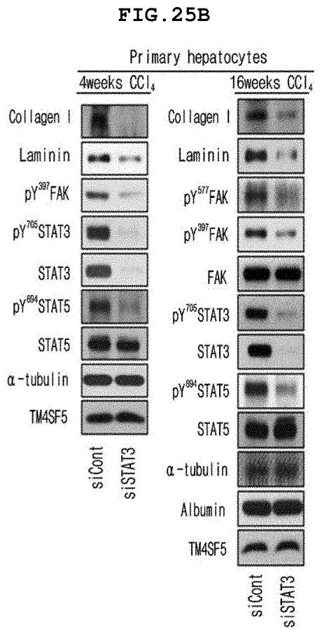

As a result, as shown in FIG. 25, the expression of laminin protein was suppressed as the expressions of TM4SF5 and STAT3 proteins were suppressed. On the other hand, when the expression of STAT3 protein was suppressed, the expression of TM4SF5 protein was not significantly changed (FIG. 25).

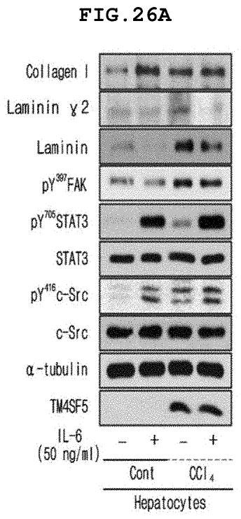

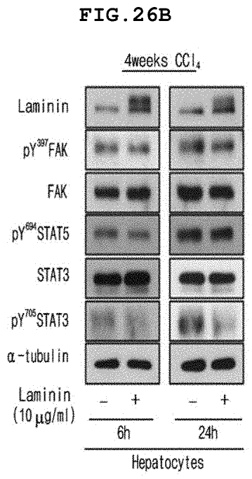

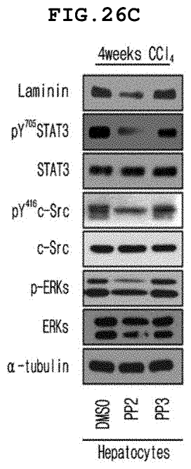

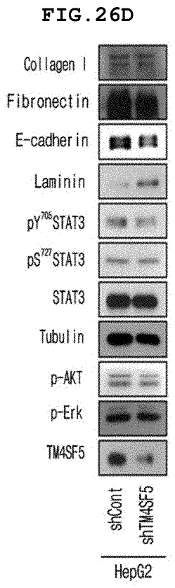

In addition, the separated liver tissue was treated with IL-6 and Western blotting was performed as described above to confirm whether the increased STAT3 phosphorylation and laminin protein expression were dependent on IL-6. As a result, as shown in FIG. 26A, the phosphorylation of STAT3 protein and the expression of collagen 1 were increased by IL-6, but the level of laminin protein was not changed (FIG. 26A). Therefore, it was confirmed that the expressions of laminin and laminin γ2 were increased depending on TM4SF5 protein.

In order to confirm the position of laminin in the signal transduction mechanism as described above, Western blotting was performed as described above by treating laminin to the separated liver tissue. As a result, as shown in FIG. 26B, the expression level of STAT3 protein was not changed by laminin (FIG. 26B). Therefore, it was confirmed that TM4SF5 protein regulated the expression of laminin through the phosphorylation of STAT3 protein.

The c-Src protein inhibitor PP2 (LC Laboratories, USA) or the control compound PP3 (LC Laboratories, USA) was added to the separated liver tissue, and the expression changes of the protein were confirmed by Western blotting as described above. As a result, as shown in FIG. 26C, the phosphorylation of STAT3 protein and the expression of laminin protein were suppressed by PP2 (FIG. 26C).

In addition, the phosphorylation of STAT3 protein and the expression changes of laminin protein in the HepG2 (Korean Cell Line Bank, Seoul) liver cancer cells in which TM4SF5 protein was suppressed were confirmed by Western blotting as described above. As a result, as shown in FIG. 26D, the phosphorylation of STAT3 protein and the expression of laminin were suppressed by the inhibition TM4SF5 protein expression (FIG. 26D).

### Example 20: Confirmation of Control Mechanism of Laminin Protein by Phosphorylation of STAT3 Protein

Whether the phosphorylation of STAT3 protein confirmed to regulate the expression changes of laminin protein controls the expression through the promoter of laminin was investigated by luciferase assay.

First, the regions corresponding to −1871 to +388 (1 kb) and −592 to +388 (2.3 kb) of LAMC2 promoter and −2865 to +85 (0.9 kb), −2047 to +89 (2.1 kb) and −845 to +89 (2.9 kb) of COL1A1 promoter were amplified by PCR using the primers listed in Table 11 below.

Table 11

| Name | Sequence (5′→3′) | SEQ. ID. NO: |
|---|---|---|
| LAMC2-0.9kb_F | AATCCTAAGTCTATAGCAGG | SEQ. ID. NO: |
| | | 97 |
| LAMC2-0.9kb_R | CCTCGATCAGGTGTTTTATGC | SEQ. ID. NO: |
| | | 98 |
| LAMC2-2.3kb_F | AGTGACTAGTGGGTTTTTTC | SEQ. ID. NO: |
| | | 99 |
| LAMC2-2.3kb_R | CCTCGATCAGGTGTTTTATC | SEQ. ID. NO: |
| | | 100 |
| COL1A1-0.9kb_F | AGGAGGTCAGAGAAGAATTT | SEQ. ID. NO: |
| | | 101 |
| COL1A1-0.9kb_R | TAGACATGTAGACTCTTTGC | SEQ. ID. NO: |
| | | 102 |
| COL1A1-2.1kb_F | AACAAAGGGTGAGCAGATCA | SEQ. ID. NO: |
| | | 103 |
| COL1A1-2.1kb_R | TAGACATGTAGACTCTTTGC | SEQ. ID. NO: |
| | | 104 |
| COL1A1-2.9kb_F | ACATTTATACCTAGGCTGCC | SEQ. ID. NO: |
| | | 105 |
| COL1A1-2.9kb_R | TAGACATGTAGACTCTTTGC | SEQ. ID. NO: |
| | | 106 |

A construct was prepared by inserting the amplified PCR product into pGL3 vector (Promega, Cat #.E1751, USA) (FIG. 27A). On the other hand, AML12 cells were cultured in a 48-well plate, which were transfected with each of the prepared construct and the constructs expressing TM4SF5 or STAT3 using lipofectamine 3000, respectively. After 24 hours, the luciferase activity was measured using a luciferase reporter assay kit (Promega, USA) according to the manufacturer's protocol.

As a result, the luciferase activity showing the promoter activity of laminin γ2 (Lamc2, FIG. 27B) or collagen I A1 (Col1a1, FIG. 27C) was increased by the expression of TM4SF5 or STAT3 protein in murine hepatocytes (AML12, FIGS. 27B and 27C) or human hepatic stellate cells (LX2, FIGS. 27B and 27C).

### Example 21: Confirmation of Type of ECM Expressed by Increase of TM4SF5 Protein Expression

It is generally known that the disease is exacerbated by the accumulation of collagen activated by hepatic stellate cells. In addition, since the luciferase activity level of collagen I and laminin γ2 was different, confirmed by the above experiment, it was expected that different types of ECM would be expressed according to cell types, so the following experiment was performed.

First, fluorescent staining was performed as described above using the liver cirrhosis tissue. As a result, it was confirmed that laminin protein was expressed around the damaged liver tissue as the expression of TM4SF5 protein increased (FIG. 28A).

In addition, the hepatocyte marker albumin and the hepatic stellate cell marker α-SMA were stained along with collagen I and laminin in the same manner as described above, in order to more clearly identify the kind of the cells. As a result, as shown in FIGS. 28B and 28C, collagen I was stained with α-SMA, laminin was initially stained with α-SMA and albumin, and then stained only with albumin when liver cirrhosis worsened (FIGS. 28B and 28C). From the above results, it was confirmed that laminin was more expressed in hepatocytes than hepatic stellate cells in a pattern different from collagen, and affected liver cirrhosis.

On the other hand, after suppressing the expression of TM4SF5 protein in the same manner as described in Example <4-1> in HepG2 cells, the expression changes of the protein was confirmed in the same manner as described above. As a result, as shown in FIGS. 28D and 28E, it was confirmed that the expression level of collagen was increased but the expression level of laminin was not increased in the cells with low expression of TM4SF5 even though the hepatic stellate cell culture medium (conditioned medium) was treated thereto or HepG2 cells and hepatic stellate cells were co-cultured in transwell chambers (Corning, USA, Hepatic stellate cells were cultured in the upper chamber and hepatic epithelial cells were cultured in the lower chamber.). Therefore, it was confirmed that laminin was regulated through the phosphorylation of STAT3 in relation to TM4SF5 in hepatic epithelial cells (FIGS. 28D and 28E).

### Example 22: Confirmation of Effect of Alleviating Liver Cirrhosis by Suppression of Laminin and Collagen Gene

It was confirmed that the expression of laminin protein was regulated by STAT3 protein. First, siRNA for laminin γ2 (LAMC2) or collagen I (COL1A1) gene was injected into the tail vein of the mouse, and CCl₄ was administered. The liver tissue was obtained from the mouse, which was stained by H&E staining. As a result, the liver damage caused by CCl₄ was suppressed (FIG. 29A). In addition, it was confirmed that the expression of TM4SF5, laminin γ2 (LAMC2) or collagen I α1 (COL1A1) protein and the phosphorylation of STAT3 were decreased (FIG. 29B), and the expression level of TM4SF5, laminin γ2 (LAMC2), collagen I α1 (COL1A1), a-SMA or TGFβ1 [symbol] mRNA was decreased (FIG. 29C).

### Example 23: Confirmation of Laminin Regulation by TM4SF5 Protein in Liver Cancer Animal Model

In the liver cancer animal model induced through fatty liver, liver cirrhosis, steatohepatitis and cirrhosis, it was confirmed whether the above-mentioned signaling was applied by the following method.

Particularly, the 52-week-old FVB/N animal model over-expressing TM4SF5 protein was bred for 1 year, and then sacrificed to extract the liver tissue. It was confirmed that TM4SF5 protein was over-expressed and nodules were formed in the extracted liver tissue (FIG. 30A). The expressions of CD34, AFP, AFU, phosphorylated STAT3, laminin, laminin γ2 and collagen I, the liver cancer markers, were increased in the liver tissue (FIGS. 30B and 30E). On the other hand, the expression level of mRNA was confirmed using the liver tissue. As a result, the expressions of the fatty liver-related genes were not increased (FIG. 30C). It was also confirmed that the expressions of CD34, HIF1α, Ki67 and cyclin D genes, the liver cancer markers, were increased with the expression of HIF1-α in the liver tissue (FIG. 30D). In addition, blood samples were analyzed. As a result, it was confirmed that the levels of AST, ALT, LDL and triglyceride were increased (FIG. 30E).

### Example 24: Confirmation of Expression Changes of TM4SF5 Protein and TM4SF5-Related Proteins in Animal Models of Liver Fibrosis and Liver Cancer

The exacerbation process of liver disease was confirmed using the transgenic mouse as follows. Particularly, the transgenic mouse was induced with liver cancer by injecting diethylnitrosamine (DEN). The liver tissue was extracted from the mouse, and H&E staining was performed. As a result, it was confirmed that liver cancer was induced (FIG. 31A). It was also confirmed that the phosphorylation of STAT3 protein and the expression of laminin were increased as the expression of TM4SF5 protein increased (FIG. 31B).

In addition, immunostaining was performed using the obtained liver tissue. As a result, it was confirmed that the expressions of TM4SF5, phosphorylated STAT3, laminins, laminin γ2 and collagen I proteins were increased (FIG. 31C).

### Example 25: Confirmation of Expression Changes of TM4SF5 Protein in Cancer Tissue of Liver Cancer Patient

The cancer tissue and the cancer surrounding tissue were obtained from liver cancer patients, and the expression changes of phosphorylated STAT3, laminin and collagen I were confirmed in the same manner as described above. At this time, the cancer surrounding tissue was a tissue at the stage before the onset of cancer, and it was expected to show the pathological symptoms of hepatitis, fibrosis and liver cirrhosis. As a result, as shown in FIG. 32, the expressions of TM4SF5, phosphorylated STAT3, laminin and collagen I were increased in the cancerous tissue and the cancer surrounding tissue (FIG. 32).

1-19. (canceled)

20. A method of diagnosing of nonalcoholic fatty liver or (fibrosis-associated) nonalcoholic steatohepatitis comprising the following steps:
1) selecting a sample obtained from a suspected nonalcoholic fatty liver or nonalcoholic steatohepatitis cell, animal, or patient in which the transcription level of transmembrane 4 L6 family member (TM4SF5) gene or the expression level of TM4SF5 protein is increased as compared to a sample obtained from a normal control group;
2) measuring an expression level of sterol regulatory element-binding transcription factor 1 (SREBP1) mRNA or protein, and/or the phosphorylation level of signal transducer and activator of transcription 3 (STAT3) protein in the sample selected in step 1); and
3) comparing the expression level of SREBP1 mRNA or protein, and/or the phosphorylation level of STAT3 protein measured in step 2) with the expression level of SREBP1 mRNA or protein, and/or the phosphorylation level of STAT3, respectively, of a normal control group sample.

21. The method of diagnosing of nonalcoholic fatty liver or nonalcoholic steatohepatitis according to claim 20, wherein the expression level of SREBP1 mRNA or protein is regulated by SIRT1 (NAD-dependent deacetylase sirtuin-1).

22. The method of diagnosing of nonalcoholic fatty liver according to claim 20, wherein the expression level of SREBP1 mRNA or protein is increased compared to the normal control group, thereby determining the presence of nonalcoholic fatty liver in the animal or patient.

23. The method of diagnosing of nonalcoholic steatohepatitis according to claim 20, wherein a decrease in the expression level of SREBP1 mRNA or as compared to the normal control group indicates that the animal or patient has nonalcoholic steatohepatitis.

24. The method of diagnosing of nonalcoholic fatty liver or nonalcoholic steatohepatitis according to claim 20, wherein the phosphorylation level of STAT3 protein is regulated by SOCS1 or SOCS3 protein.

25. The method of diagnosing of nonalcoholic fatty liver according to claim 20, wherein a decrease in the phosphorylation level of STAT3 protein as compared to the normal control group indicates that the animal or patient has nonalcoholic fatty liver.

26. The method of diagnosing of nonalcoholic steatohepatitis according to claim 20, wherein an increase in the phosphorylation level of STAT3 protein as compared to the normal control group indicates that the animal or patient has nonalcoholic steatohepatitis.

27. The method of diagnosing of nonalcoholic fatty liver according to claim 22 or claim 25, further comprising a step of measuring the expression of one or more mRNAs or proteins of SREBP1c, SREBP2, CD36, fatty acid-binding protein 1 (FABP1), fatty Acid Synthase (FASN), Acetyl-CoA carboxylase (ACC)α, Accβ, low density lipoprotein receptor (LDLR), very Low Density Lipoprotein Receptor (VLDLR), proliferator-activated receptors (PPAR) γ, PPARα, ApoB100, and Leptin.

28. The method of diagnosing of nonalcoholic steatohepatitis or fibrosis-associated nonalcoholic steatohepatitis according to claim 23 or claim 26, further comprising a step of measuring the expression level of extracellular matrix (ECM), wherein the ECM comprises one or more of α-SMA (α-smooth muscle actin), albumin, Vimentin, collagen, laminin or laminin γ2.

29. The method of diagnosing of nonalcoholic steatohepatitis according to claim 28, wherein and increase in the expression level of extracellular matrix as compared to the normal control group indicates that the animal or patient has nonalcoholic steatohepatitis.

30. The method of diagnosing of nonalcoholic steatohepatitis or fibrosis-associated nonalcoholic steatohepatitis according to claim 23 or claim 26, further comprising a step of comparing the expression levels of one or more of mRNAs or proteins of collagen I, α-smooth muscle actin (α-SMA), interleukin (IL)-6, transforming growth factor beta (TGβ)1, vimentin, tissue inhibitor of metalloproteinase (TIMP)1, tumor necrosis factor (TNF)α, monocyte chemotactic protein (MCP) 1 (CCL2)], and F4/80 antigen to the normal control group.

31. The method of diagnosing of nonalcoholic steatohepatitis according to claim 30, wherein an increase of the expression levels of one or more mRNAs or proteins of collagen I, α-SMA, IL-6, TGFβ1, vimentin, TIMP1, TNFα, MCP1, and F4/80 antigen as compared to the normal control group indicates that the animal or patient has nonalcoholic steatohepatitis.

32. A method for screening a candidate substance in vitro for treatment of nonalcoholic fatty liver comprising the following steps:
1) treating the cells expressing TM4SF5 protein with a test substance for treating nonalcoholic fatty liver to in vitro;
2) measuring an expression level of SREBP1 mRNA or protein and/or the phosphorylation level of STAT3 protein in the cells of step 1); and
3) selecting a test substance that suppresses the expression of TM4SF5 protein, the expression level of SREBP1 mRNA or protein, and/or increases the phosphorylation level of STAT3 protein, compared to a control group not treated with the test substance of step 1), thereby selecting an candidate substance in vitro for treatment nonalcoholic fatty liver.

33. A method for screening a candidate substance for treatment of nonalcoholic fatty liver or nonalcoholic steatohepatitis comprising the following steps:
1) treating transgenic mouse expressing TM4SF5 protein with a test substance for treating nonalcoholic fatty liver or nonalcoholic steatohepatitis;
2) measuring an expression level of SREBP1 mRNA or protein and/or the phosphorylation level of STAT3 protein in the transgenic mouse of step 1); and
3) selecting a test substance that suppresses the expression of TM4SF5 protein, the expression level of SREBP1 mRNA or protein, reduces the synthesis of fatty acid, cholesterol, monoacyl-, diacyl- or triacyl-glycerol, and/or reduces liver/body or body weight of the transgenic mouse as compared to a control mouse not treated with the test substance of step 1), thereby selecting the candidate substance.

34. A method for screening a candidate substance in vitro for treating nonalcoholic steatohepatitis comprising the following steps:
1) treating cells expressing TM4SF5 protein with a test substance for treating nonalcoholic steatohepatitis in vitro;
2) measuring the expression level of SREBP1 mRNA or protein and/or the phosphorylation level of STAT3 protein in the cells of step 1); and
3) selecting a test substance that suppresses the expression of TM4SF5 protein, increases the expression level of SREBP1 mRNA or protein, and/or suppresses the phosphorylation level of STAT3 protein, as compared to a control group not treated with the test substance of step 1), thereby selecting the candidate substance.

35. A method for screening a candidate substance for treating nonalcoholic steatohepatitis comprising the following steps:
1) treating a transgenic mouse or patient expressing TM4SF5 protein with a test substance for treating nonalcoholic steatohepatitis to the;
2) measuring the expression level of SREBP1 mRNA or protein and/or the phosphorylation level of STAT3 protein in the transgenic mouse or patient of step 1); and
3) selecting a test substance that suppresses the expression of TM4SF5 protein, increases the expression level of SREBP1 mRNA or protein, and/or suppresses the phosphorylation level of STAT3 protein, as compared to a control group of transgenic mice or patients, respectively, not treated with the test substance of step 1), thereby selecting the candidate substance.


SEQUENCE LISTING (112 sequences)

SEQ ID NO: 1 - protein, 197 aa, Homo sapiens
MCTGKCARCVGLSLITLCLVCIVANALLLVPNGETSWTNTNHLSLQVWLMGGFIGGGLMVLCPGIAAVRAGGKGCCGAGCCGNRCRMLRSVFSSAFGVLGAIYCLSVSGAGLRNGPRCLMNGEWGYHFEDTAGAYLLNRTLWDRCEAPPRVVPWNVTLFSLLVAASCLEIVLCGIQLVNATIGVFCGDCRKKQDTPH

SEQ ID NO: 2 - protein, 629 aa, Homo sapiens
MGCKVLLNIGQQMLRRKVVDCSREETRLSRCLNTFDLVALGVGSTLGAGVYVLAGAVARENAGPAIVISFLIAALASVLAGLCYGEFGARVPKTGSAYLYSYVTVGELWAFITGWNLILSYIIGTSSVARAWSATFDELIGRPIGEFSRTHMTLNAPGVLAENPDIFAVIIILILTGLLTLGVKESAMVNKIFTCINVLVLGFIMVSGFVKGSVKNWQLTEEDFGNTSGRLCLNNDTKEGKPGVGGFMPFGFSGVLSGAATCFYAFVGFDCIATTGEEVKNPQKAIPVGIVASLLICFIAYFGVSAALTLMMPYFCLDNNSPLPDAFKHVGWEGAKYAVAVGSLCALSASLLGSMFPMPRVIYAMAEDGLLFKFLANVNDRTKTPIIATLASGAVAAVMAFLFDLKDLVDLMSIGTLLAYSLVAACVLVLRYQPEQPNLVYQMASTSDELDPADQNELASTNDSQLGFLPEAEMFSLKTILSPKNMEPSKISGLIVNISTSLIAVLIITFCIVTVLGREALTKGALWAVFLLAGSALLCAVVTGVIWRQPESKTKLSFKVPFLPVLPILSIFVNVYLMMQLDQGTWVRFAVWMLIGFIIYFGYGLWHSEEASLDADQARTPDGNLDQCK

SEQ ID NO: 3 - DNA, 708 bp, Homo sapiens
actcaccgcctgtccttcctgacacctcaccatgtgtacgggaaaatgtgcccgctgtgtggggctctccctcattaccctctgcctcgtctgcattgtggccaacgccctcctgctggtacctaatggggagacctcctggaccaacaccaaccatctcagcttgcaagtctggctcatgggcggcttcattggcgggggcctaatggtactgtgtccggggattgcagccgttcgggcagggggcaagggctgctgtggtgctgggtgctgtggaaaccgctgcaggatgctgcgctcggtcttctcctcggcgttcggggtgcttggtgccatctactgcctctcggtgtctggagctgggctccgaaatggacccagatgcttaatgaacggcgagtggggctaccacttcgaagacaccgcgggagcttacttgctcaaccgcactctatgggatcggtgcgaggcgccccctcgcgtggtcccctggaatgtgacgctcttctcgctgctggtggccgcctcctgcctggagatagtactgtgtgggatccagctggtgaacgcgaccattggtgtcttctgcggcgattgcaggaaaaaacaggacacacctcactgaggctccactgaccgccgggttacacctgctccttcctggacgctcactcccttgctcgctagaataaactgctttgcgctctc

SEQ ID NO: 4 - DNA, 8972 bp, Homo sapiens
gtccaagggtagccaaggatggctgcagcttcatatgatcagttgttaaagcaagttgaggcactgaagatggagaactcaaatcttcgacaagagctagaagataattccaatcatcttacaaaactggaaactgaggcatctaatatgaaggaagtacttaaacaactacaaggaagtattgaagatgaagctatggcttcttctggacagattgatttattagagcgtcttaaagagcttaacttagatagcagtaatttccctggagtaaaactgcggtcaaaaatgtccctccgttcttatggaagccgggaaggatctgtatcaagccgttctggagagtgcagtcctgttcctatgggttcatttccaagaagagggtttgtaaatggaagcagagaaagtactggatatttagaagaacttgagaaagagaggtcattgcttcttgctgatcttgacaaagaagaaaaggaaaaagactggtattacgctcaacttcagaatctcactaaaagaatagatagtcttcctttaactgaaaatttttccttacaaacagatatgaccagaaggcaattggaatatgaagcaaggcaaatcagagttgcgatggaagaacaactaggtacctgccaggatatggaaaaacgagcacagcgaagaatagccagaattcagcaaatcgaaaaggacatacttcgtatacgacagcttttacagtcccaagcaacagaagcagagaggtcatctcagaacaagcatgaaaccggctcacatgatgctgagcggcagaatgaaggtcaaggagtgggagaaatcaacatggcaacttctggtaatggtcagggttcaactacacgaatggaccatgaaacagccagtgttttgagttctagtagcacacactctgcacctcgaaggctgacaagtcatctgggaaccaaggtggaaatggtgtattcattgttgtcaatgcttggtactcatgataaggatgatatgtcgcgaactttgctagctatgtctagctcccaagacagctgtatatccatgcgacagtctggatgtcttcctctcctcatccagcttttacatggcaatgacaaagactctgtattgttgggaaattcccggggcagtaaagaggctcgggccagggccagtgcagcactccacaacatcattcactcacagcctgatgacaagagaggcaggcgtgaaatccgagtccttcatcttttggaacagatacgcgcttactgtgaaacctgttgggagtggcaggaagctcatgaaccaggcatggaccaggacaaaaatccaatgccagctcctgttgaacatcagatctgtcctgctgtgtgtgttctaatgaaactttcatttgatgaagagcatagacatgcaatgaatgaactagggggactacaggccattgcagaattattgcaagtggactgtgaaatgtacgggcttactaatgaccactacagtattacactaagacgatatgctggaatggctttgacaaacttgacttttggagatgtagccaacaaggctacgctatgctctatgaaaggctgcatgagagcacttgtggcccaactaaaatctgaaagtgaagacttacagcaggttattgcaagtgttttgaggaatttgtcttggcgagcagatgtaaatagtaaaaagacgttgcgagaagttggaagtgtgaaagcattgatggaatgtgctttagaagttaaaaaggaatcaaccctcaaaagcgtattgagtgccttatggaatttgtcagcacattgcactgagaataaagctgatatatgtgctgtagatggtgcacttgcatttttggttggcactcttacttaccggagccagacaaacactttagccattattgaaagtggaggtgggatattacggaatgtgtccagcttgatagctacaaatgaggaccacaggcaaatcctaagagagaacaactgtctacaaactttattacaacacttaaaatctcatagtttgacaatagtcagtaatgcatgtggaactttgtggaatctctcagcaagaaatcctaaagaccaggaagcattatgggacatgggggcagttagcatgctcaagaacctcattcattcaaagcacaaaatgattgctatgggaagtgctgcagctttaaggaatctcatggcaaataggcctgcgaagtacaaggatgccaatattatgtctcctggctcaagcttgccatctcttcatgttaggaaacaaaaagccctagaagcagaattagatgctcagcacttatcagaaacttttgacaatatagacaatttaagtcccaaggcatctcatcgtagtaagcagagacacaagcaaagtctctatggtgattatgtttttgacaccaatcgacatgatgataataggtcagacaattttaatactggcaacatgactgtcctttcaccatatttgaatactacagtgttacccagctcctcttcatcaagaggaagcttagatagttctcgttctgaaaaagatagaagtttggagagagaacgcggaattggtctaggcaactaccatccagcaacagaaaatccaggaacttcttcaaagcgaggtttgcagatctccaccactgcagcccagattgccaaagtcatggaagaagtgtcagccattcatacctctcaggaagacagaagttctgggtctaccactgaattacattgtgtgacagatgagagaaatgcacttagaagaagctctgctgcccatacacattcaaacacttacaatttcactaagtcggaaaattcaaataggacatgttctatgccttatgccaaattagaatacaagagatcttcaaatgatagtttaaatagtgtcagtagtagtgatggttatggtaaaagaggtcaaatgaaaccctcgattgaatcctattctgaagatgatgaaagtaagttttgcagttatggtcaatacccagccgacctagcccataaaatacatagtgcaaatcatatggatgataatgatggagaactagatacaccaataaattatagtcttaaatattcagatgagcagttgaactctggaaggcaaagtccttcacagaatgaaagatgggcaagacccaaacacataatagaagatgaaataaaacaaagtgagcaaagacaatcaaggaatcaaagtacaacttatcctgtttatactgagagcactgatgataaacacctcaagttccaaccacattttggacagcaggaatgtgtttctccatacaggtcacggggagccaatggttcagaaacaaatcgagtgggttctaatcatggaattaatcaaaatgtaagccagtctttgtgtcaagaagatgactatgaagatgataagcctaccaattatagtgaacgttactctgaagaagaacagcatgaagaagaagagagaccaacaaattatagcataaaatataatgaagagaaacgtcatgtggatcagcctattgattatagtttaaaatatgccacagatattccttcatcacagaaacagtcattttcattctcaaagagttcatctggacaaagcagtaaaaccgaacatatgtcttcaagcagtgagaatacgtccacaccttcatctaatgccaagaggcagaatcagctccatccaagttctgcacagagtagaagtggtcagcctcaaaaggctgccacttgcaaagtttcttctattaaccaagaaacaatacagacttattgtgtagaagatactccaatatgtttttcaagatgtagttcattatcatctttgtcatcagctgaagatgaaataggatgtaatcagacgacacaggaagcagattctgctaataccctgcaaatagcagaaataaaagaaaagattggaactaggtcagctgaagatcctgtgagcgaagttccagcagtgtcacagcaccctagaaccaaatccagcagactgcagggttctagtttatcttcagaatcagccaggcacaaagctgttgaattttcttcaggagcgaaatctccctccaaaagtggtgctcagacacccaaaagtccacctgaacactatgttcaggagaccccactcatgtttagcagatgtacttctgtcagttcacttgatagttttgagagtcgttcgattgccagctccgttcagagtgaaccatgcagtggaatggtaagtggcattataagccccagtgatcttccagatagccctggacaaaccatgccaccaagcagaagtaaaacacctccaccacctcctcaaacagctcaaaccaagcgagaagtacctaaaaataaagcacctactgctgaaaagagagagagtggacctaagcaagctgcagtaaatgctgcagttcagagggtccaggttcttccagatgctgatactttattacattttgccacggaaagtactccagatggattttcttgttcatccagcctgagtgctctgagcctcgatgagccatttatacagaaagatgtggaattaagaataatgcctccagttcaggaaaatgacaatgggaatgaaacagaatcagagcagcctaaagaatcaaatgaaaaccaagagaaagaggcagaaaaaactattgattctgaaaaggacctattagatgattcagatgatgatgatattgaaatactagaagaatgtattatttctgccatgccaacaaagtcatcacgtaaagcaaaaaagccagcccagactgcttcaaaattacctccacctgtggcaaggaaaccaagtcagctgcctgtgtacaaacttctaccatcacaaaacaggttgcaaccccaaaagcatgttagttttacaccgggggatgatatgccacgggtgtattgtgttgaagggacacctataaacttttccacagctacatctctaagtgatctaacaatcgaatcccctccaaatgagttagctgctggagaaggagttagaggaggagcacagtcaggtgaatttgaaaaacgagataccattcctacagaaggcagaagtacagatgaggctcaaggaggaaaaacctcatctgtaaccatacctgaattggatgacaataaagcagaggaaggtgatattcttgcagaatgcattaattctgctatgcccaaagggaaaagtcacaagcctttccgtgtgaaaaagataatggaccaggtccagcaagcatctgcgtcgtcttctgcacccaacaaaaatcagttagatggtaagaaaaagaaaccaacttcaccagtaaaacctataccacaaaatactgaatataggacacgtgtaagaaaaaatgcagactcaaaaaataatttaaatgctgagagagttttctcagacaacaaagattcaaagaaacagaatttgaaaaataattccaaggacttcaatgataagctcccaaataatgaagatagagtcagaggaagttttgcttttgattcacctcatcattacacgcctattgaaggaactccttactgtttttcacgaaatgattctttgagttctctagattttgatgatgatgatgttgacctttccagggaaaaggctgaattaagaaaggcaaaagaaaataaggaatcagaggctaaagttaccagccacacagaactaacctccaaccaacaatcagctaataagacacaagctattgcaaagcagccaataaatcgaggtcagcctaaacccatacttcagaaacaatccacttttccccagtcatccaaagacataccagacagaggggcagcaactgatgaaaagttacagaattttgctattgaaaatactccagtttgcttttctcataattcctctctgagttctctcagtgacattgaccaagaaaacaacaataaagaaaatgaacctatcaaagagactgagccccctgactcacagggagaaccaagtaaacctcaagcatcaggctatgctcctaaatcatttcatgttgaagataccccagtttgtttctcaagaaacagttctctcagttctcttagtattgactctgaagatgacctgttgcaggaatgtataagctccgcaatgccaaaaaagaaaaagccttcaagactcaagggtgataatgaaaaacatagtcccagaaatatgggtggcatattaggtgaagatctgacacttgatttgaaagatatacagagaccagattcagaacatggtctatcccctgattcagaaaattttgattggaaagctattcaggaaggtgcaaattccatagtaagtagtttacatcaagctgctgctgctgcatgtttatctagacaagcttcgtctgattcagattccatcctttccctgaaatcaggaatctctctgggatcaccatttcatcttacacctgatcaagaagaaaaaccctttacaagtaataaaggcccacgaattctaaaaccaggggagaaaagtacattggaaactaaaaagatagaatctgaaagtaaaggaatcaaaggaggaaaaaaagtttataaaagtttgattactggaaaagttcgatctaattcagaaatttcaggccaaatgaaacagccccttcaagcaaacatgccttcaatctctcgaggcaggacaatgattcatattccaggagttcgaaatagctcctcaagtacaagtcctgtttctaaaaaaggcccaccccttaagactccagcctccaaaagccctagtgaaggtcaaacagccaccacttctcctagaggagccaagccatctgtgaaatcagaattaagccctgttgccaggcagacatcccaaataggtgggtcaagtaaagcaccttctagatcaggatctagagattcgaccccttcaagacctgcccagcaaccattaagtagacctatacagtctcctggccgaaactcaatttcccctggtagaaatggaataagtcctcctaacaaattatctcaacttccaaggacatcatcccctagtactgcttcaactaagtcctcaggttctggaaaaatgtcatatacatctccaggtagacagatgagccaacagaaccttaccaaacaaacaggtttatccaagaatgccagtagtattccaagaagtgagtctgcctccaaaggactaaatcagatgaataatggtaatggagccaataaaaaggtagaactttctagaatgtcttcaactaaatcaagtggaagtgaatctgatagatcagaaagacctgtattagtacgccagtcaactttcatcaaagaagctccaagcccaaccttaagaagaaaattggaggaatctgcttcatttgaatctctttctccatcatctagaccagcttctcccactaggtcccaggcacaaactccagttttaagtccttcccttcctgatatgtctctatccacacattcgtctgttcaggctggtggatggcgaaaactcccacctaatctcagtcccactatagagtataatgatggaagaccagcaaagcgccatgatattgcacggtctcattctgaaagtccttctagacttccaatcaataggtcaggaacctggaaacgtgagcacagcaaacattcatcatcccttcctcgagtaagcacttggagaagaactggaagttcatcttcaattctttctgcttcatcagaatccagtgaaaaagcaaaaagtgaggatgaaaaacatgtgaactctatttcaggaaccaaacaaagtaaagaaaaccaagtatccgcaaaaggaacatggagaaaaataaaagaaaatgaattttctcccacaaatagtacttctcagaccgtttcctcaggtgctacaaatggtgctgaatcaaagactctaatttatcaaatggcacctgctgtttctaaaacagaggatgtttgggtgagaattgaggactgtcccattaacaatcctagatctggaagatctcccacaggtaatactcccccggtgattgacagtgtttcagaaaaggcaaatccaaacattaaagattcaaaagataatcaggcaaaacaaaatgtgggtaatggcagtgttcccatgcgtaccgtgggtttggaaaatcgcctgaactcctttattcaggtggatgcccctgaccaaaaaggaactgagataaaaccaggacaaaataatcctgtccctgtatcagagactaatgaaagttctatagtggaacgtaccccattcagttctagcagctcaagcaaacacagttcacctagtgggactgttgctgccagagtgactccttttaattacaacccaagccctaggaaaagcagcgcagatagcacttcagctcggccatctcagatcccaactccagtgaataacaacacaaagaagcgagattccaaaactgacagcacagaatccagtggaacccaaagtcctaagcgccattctgggtcttaccttgtgacatctgtttaaaagagaggaagaatgaaactaagaaaattctatgttaattacaactgctatatagacattttgtttcaaatgaaactttaaaagactgaaaaattttgtaaataggtttgattcttgttagagggtttttgttctggaagccatatttgatagtatactttgtcttcactggtcttattttgggaggcactcttgatggttaggaaaaaaatagtaaagccaagtatgtttgtacagtatgttttacatgtatttaaagtagcacccatcccaacttcctttaattattgcttgtcttaaaataatgaacactacagatagaaaatatgatatattgctgttatcaatcatttctagattataaactgactaaacttacatcagggaaaaattggtatttatgcaaaaaaaaatgtttttgt

SEQ ID NO: 5 - DNA, 21 bp, Artificial
  CMV forward
cgctattaccatggtgatgcg

SEQ ID NO: 6 - DNA, 21 bp, Artificial
  TM4SF5 reverse
agacaccgagaggcagtagat

SEQ ID NO: 7 - DNA, 21 bp, Artificial
  Srebp1_F
catcgactacatccgcttctt

SEQ ID NO: 8 - DNA, 21 bp, Artificial
  Srebp1_R
caccaggtccttcagtgattt

SEQ ID NO: 9 - DNA, 19 bp, Artificial
  Srebp2_F
tggatgacgcaaaggtcaa

SEQ ID NO: 10 - DNA, 22 bp, Artificial
  Srebp2_R
caggaaggtgaggacacataag

SEQ ID NO: 11 - DNA, 20 bp, Artificial
  Cd36_F
ttggccaagctattgcgaca

SEQ ID NO: 12 - DNA, 20 bp, Artificial
  Cd36_R
ctggaggggtgatgcaaagg

SEQ ID NO: 13 - DNA, 20 bp, Artificial
  Fabp1_F
cccgaggacctcatccagaa

SEQ ID NO: 14 - DNA, 20 bp, Artificial
  Fabp1_R
ccccagggtgaactcattgc

SEQ ID NO: 15 - DNA, 20 bp, Artificial
  Fasn_F
tctgggccaacctcattggt

SEQ ID NO: 16 - DNA, 20 bp, Artificial
  Fasn_R
gaagctgggggtccattgtg

SEQ ID NO: 17 - DNA, 21 bp, Artificial
  Acc alpha_F
acattccgagcaagggataag

SEQ ID NO: 18 - DNA, 20 bp, Artificial
  Acc alpha_R
gggatggcagtaaggtcaaa

SEQ ID NO: 19 - DNA, 22 bp, Artificial
  Acc beta_F
gtcctgcccactttcttctatc

SEQ ID NO: 20 - DNA, 22 bp, Artificial
  Acc beta_R
gtttagctcgtaggcgatgtag

SEQ ID NO: 21 - DNA, 19 bp, Artificial
  Tm4sf5_F
gtcttctcctccgcctttg

SEQ ID NO: 22 - DNA, 23 bp, Artificial
  Tm4sf5_R
ggtagtcccacttgttgtctatt

SEQ ID NO: 23 - DNA, 19 bp, Artificial
  Srebp2_F
tggatgacgcaaaggtcaa

SEQ ID NO: 24 - DNA, 22 bp, Artificial
  Srebp2_R
caggaaggtgaggacacataag

SEQ ID NO: 25 - DNA, 20 bp, Artificial
  Cd36_F
ttggccaagctattgcgaca

SEQ ID NO: 26 - DNA, 20 bp, Artificial
  Cd36_R
ctggaggggtgatgcaaagg

SEQ ID NO: 27 - DNA, 20 bp, Artificial
  Fabp1_F
cccgaggacctcatccagaa

SEQ ID NO: 28 - DNA, 20 bp, Artificial
  Fabp1_R
ccccagggtgaactcattgc

SEQ ID NO: 29 - DNA, 20 bp, Artificial
  Fasn_F
tctgggccaacctcattggt

SEQ ID NO: 30 - DNA, 20 bp, Artificial
  Fasn_R
gaagctgggggtccattgtg

SEQ ID NO: 31 - DNA, 20 bp, Artificial
  Ldlr_F
gcctttgccaaaacgtcacc

SEQ ID NO: 32 - DNA, 20 bp, Artificial
  Ldlr_R
cctgaggtcccatccaatgc

SEQ ID NO: 33 - DNA, 19 bp, Artificial
  Vldlr_F
tcagtcccaggcagcgtat

SEQ ID NO: 34 - DNA, 19 bp, Artificial
  Vldlr_R
cttgatcttggcgggtgtt

SEQ ID NO: 35 - DNA, 29 bp, Artificial
  shTM4SF5
cctggaatgtgacgctcttctcgctgctg

SEQ ID NO: 36 - DNA, 19 bp, Artificial
  Tm4sf5_F
gtcttctcctccgcctttg

SEQ ID NO: 37 - DNA, 23 bp, Artificial
  Tm4sf5_R
ggtagtcccacttgttgtctatt

SEQ ID NO: 38 - DNA, 21 bp, Artificial
  Srebp1_F
catcgactacatccgcttctt

SEQ ID NO: 39 - DNA, 21 bp, Artificial
  Srebp1_R
caccaggtccttcagtgattt

SEQ ID NO: 40 - DNA, 20 bp, Artificial
  Cd36_F
ttggccaagctattgcgaca

SEQ ID NO: 41 - DNA, 20 bp, Artificial
  Cd36_R
ctggaggggtgatgcaaagg

SEQ ID NO: 42 - DNA, 20 bp, Artificial
  Fabp1_F
cccgaggacctcatccagaa

SEQ ID NO: 43 - DNA, 20 bp, Artificial
  Fabp1_R
ccccagggtgaactcattgc

SEQ ID NO: 44 - DNA, 20 bp, Artificial
  Fasn_F
tctgggccaacctcattggt

SEQ ID NO: 45 - DNA, 20 bp, Artificial
  Fasn_R
gaagctgggggtccattgtg

SEQ ID NO: 46 - DNA, 22 bp, Artificial
  Ppar gamma_F
ctggcctccctgatgaataaag

SEQ ID NO: 47 - DNA, 22 bp, Artificial
  Ppar gamma_R
aggctccataaagtcaccaaag

SEQ ID NO: 48 - DNA, 26 bp, Artificial
  Sirt1_F
gcatagataccgtctcttgatctgaa

SEQ ID NO: 49 - DNA, 25 bp, Artificial
  Sirt1_R
tgtgaagttactgcaggagtgtaaa

SEQ ID NO: 50 - DNA, 20 bp, Artificial
  Sirt2_F
ttccatcgcgcttcttctcc

SEQ ID NO: 51 - DNA, 20 bp, Artificial
  Sirt2_R
ccaggccacgtccctgtaag

SEQ ID NO: 52 - DNA, 20 bp, Artificial
  Sirt3_F
acctcctggggtggacacaa

SEQ ID NO: 53 - DNA, 20 bp, Artificial
  Sirt3_R
ggccccaagggtagacatcc

SEQ ID NO: 54 - DNA, 20 bp, Artificial
  Sirt4_F
agctttcaggtcccgtgctg

SEQ ID NO: 55 - DNA, 20 bp, Artificial
  Sirt4_R
tcaggcaagccaaatcgtca

SEQ ID NO: 56 - DNA, 20 bp, Artificial
  Sirt5_F
tctacccggctgccatgttt

SEQ ID NO: 57 - DNA, 20 bp, Artificial
  Sirt5_R
tgaggagcaagggcttcagg

SEQ ID NO: 58 - DNA, 20 bp, Artificial
  Sirt6_F
gggacctgatgctcgctgat

SEQ ID NO: 59 - DNA, 20 bp, Artificial
  Sirt6_R
cagaggtggcagggctttgt

SEQ ID NO: 60 - DNA, 20 bp, Artificial
  Sirt7_F
tgccaggcacttggttgtct

SEQ ID NO: 61 - DNA, 20 bp, Artificial
  Sirt7_R
taggctccgcttcgcttagg

SEQ ID NO: 62 - DNA, 19 bp, Artificial
  SOCS1_F
gggtggcaaagaaaaggag

SEQ ID NO: 63 - DNA, 20 bp, Artificial
  SOCS1_R
gttgagcgtcaagacccagt

SEQ ID NO: 64 - DNA, 22 bp, Artificial
  SOCS2_F
tccagatgtgcaaggataaacg

SEQ ID NO: 65 - DNA, 23 bp, Artificial
  SOCS2_R
aggtacaggtgaacagtcccatt

SEQ ID NO: 66 - DNA, 19 bp, Artificial
  SCOS3_F
tgcaggagagcggattcta

SEQ ID NO: 67 - DNA, 20 bp, Artificial
  SCOS3_R
agctgtcgcggataagaaag

SEQ ID NO: 68 - DNA, 21 bp, Artificial
  SCOS5_F
gagggaggaagccgtaatgag

SEQ ID NO: 69 - DNA, 19 bp, Artificial
  SCOS5_R
cggcacagttttggttccg

SEQ ID NO: 70 - DNA, 23 bp, Artificial
  RG1
gcgggagctgggctccgaattgg

SEQ ID NO: 71 - DNA, 23 bp, Artificial
  RG2
ttaagcatttgggtccaattcgg

SEQ ID NO: 72 - DNA, 23 bp, Artificial
  RG3
tgagaaatcctgtttgatcttgg

SEQ ID NO: 73 - DNA, 23 bp, Artificial
  RG4
aggtattaggggtggcctatggg

SEQ ID NO: 74 - DNA, 20 bp, Artificial
  mouse TM4SF5_forward
gtagtatgcgggaggcactg

SEQ ID NO: 75 - DNA, 20 bp, Artificial
  mouse TM4SF5_reverse
gggtgaccactcagacttcc

SEQ ID NO: 76 - DNA, 20 bp, Artificial
  Fasn_F
tctgggccaacctcattggt

SEQ ID NO: 77 - DNA, 20 bp, Artificial
  Fasn_R
gaagctgggggtccattgtg

SEQ ID NO: 78 - DNA, 22 bp, Artificial
  Ppar gamma_F
ctggcctccctgatgaataaag

SEQ ID NO: 79 - DNA, 22 bp, Artificial
  Ppar gamma_R
aggctccataaagtcaccaaag

SEQ ID NO: 80 - DNA, 20 bp, Artificial
  L-Fabp_F
tggacccaaagtggtccgca

SEQ ID NO: 81 - DNA, 20 bp, Artificial
  L-Fabp_R
agttcagtcacggactttat

SEQ ID NO: 82 - DNA, 20 bp, Artificial
  Srebf-1c_F
gtgttggcctgcttggctct

SEQ ID NO: 83 - DNA, 20 bp, Artificial
  Srebf-1c_R
gagcagcctgggggaaatct

SEQ ID NO: 84 - DNA, 20 bp, Artificial
  beta-actin_F
ggccgggacctgacagacta

SEQ ID NO: 85 - DNA, 20 bp, Artificial
  beta-actin_R
aggaagaggatgcggcagtg

SEQ ID NO: 86 - DNA, 19 bp, Artificial
  Tm4sf5_F
gtcttctcctccgcctttg

SEQ ID NO: 87 - DNA, 23 bp, Artificial
  Tm4sf5_R
ggtagtcccacttgttgtctatt

SEQ ID NO: 88 - DNA, 17 bp, Artificial
  MAPC MT
tgagaaagacagaagta

SEQ ID NO: 89 - DNA, 19 bp, Artificial
  MAPC 15
ttccactttggcataaggc

SEQ ID NO: 90 - DNA, 18 bp, Artificial
  MAPC 9
gccatcccttcacgttag

SEQ ID NO: 91 - DNA, 20 bp, Artificial
  beta-actin_F
ggccgggacctgacagacta

SEQ ID NO: 92 - DNA, 20 bp, Artificial
  beta-actin_R
aggaagaggatgcggcagtg

SEQ ID NO: 93 - protein, 478 aa, Mus musculus
MAGVEQAASFGGHLNGDLDPDDREEGTSSTAEEAAKKKRRKKKKGKGAVSAVQQELDKESGALVDEVAKQLESQALEEKERDDDDEDGDGDADGATGKKKKKKKKKRGPKVQTDPPSVPICDLYPNGVFPKGQECEYPPTQDGRTAAWRTTSEEKKALDQASEEIWNDFREAAEAHRQVRKYVMSWIKPGMTMIEICEKLEDCSRKLIKENGLNAGLAFPTGCSLNNCAAHYTPNAGDTTVLQYDDICKIDFGTHISGRIIDCAFTVTFNPKYDILLTAVKDATNTGIKCAGIDVRLCDVGEAIQEVMESYEVEIDGKTYQVKPIRNLNGHSIGPYRIHAGKTVPIVKGGEATRMEEGEVYAIETFGSTGKGVVHDDMECSHYMKNFDVGHVPIRLPRTKHLLNVINENFGTLAFCRRWLDRLGESKYLMALKNLCDLGIVDPYPPLCDIKGSYTAQFEHTILLRPTCKEVVSRGDDY

SEQ ID NO: 94 - protein, 329 aa, Homo sapiens
MELHILEHRVRVLSVARPGLWLYTHPLIKLLFLPRRSRCKFFSLTETPEDYTLMVDEEGFKELPPSEFLQVAEATWLVLNVSSHSGAAVQAAGVTKIARSVIAPLAEHHVSVLMLSTYQTDFILVREQDLSVVIHTLAQEFDIYREVGGEPVPVTRDDSSNGFPRTQHGPSPTVHPIQSPQNRFCVLTLDPETLPAIATTLIDVLFYSHSTPKEAASSSPEPSSITFFAFSLIEGYISIVMDAETQKKFPSDLLLTSSSGELWRMVRIGGQPLGFDECGIVAQIAGPLAAADISAYYISTFNFDHALVPEDGIGSVIEVLQRRQEGLAS

SEQ ID NO: 95 - RNA, 23 nt, Artificial
  shTM4SF5 #2
accauguguacgggaaaaugugc

SEQ ID NO: 96 - RNA, 19 nt, Artificial
  shTM4SF5 #4
ccaucucagcuugcaaguc

SEQ ID NO: 97 - DNA, 20 bp, Artificial
  LAMC2-0.9kb_F
aatcctaagtctatagcagg

SEQ ID NO: 98 - DNA, 21 bp, Artificial
  LAMC2-0.9kb_R
cctcgatcaggtgttttatgc

SEQ ID NO: 99 - DNA, 20 bp, Artificial
  LAMC2-2.3kb_F
agtgactagtgggttttttc

SEQ ID NO: 100 - DNA, 20 bp, Artificial
  LAMC2-2.3kb_R
cctcgatcaggtgttttatc

SEQ ID NO: 101 - DNA, 20 bp, Artificial
  COL1A1-0.9kb_F
aggaggtcagagaagaattt

SEQ ID NO: 102 - DNA, 20 bp, Artificial
  COL1A1-0.9kb_R
tagacatgtagactctttgc

SEQ ID NO: 103 - DNA, 20 bp, Artificial
  COL1A1-2.1kb_F
aacaaagggtgagcagatca

SEQ ID NO: 104 - DNA, 20 bp, Artificial
  COL1A1-2.1kb_R
tagacatgtagactctttgc

SEQ ID NO: 105 - DNA, 20 bp, Artificial
  COL1A1-2.9kb_F
acatttatacctaggctgcc

SEQ ID NO: 106 - DNA, 21 bp, Artificial
  COL1A1-2.9kb_R
cgctattaccatggtgatgcg

SEQ ID NO: 107 - DNA, 23 bp, Artificial
  RG1
gaggttgccgtccgtccaggtgg

SEQ ID NO: 108 - DNA, 23 bp, Artificial
  RG2
gctgaggttgccgtccgtccagg

SEQ ID NO: 109 - DNA, 20 bp, Artificial
  mouse TM4SF5_forward
acttcctcagggcctctctc

SEQ ID NO: 110 - DNA, 20 bp, Artificial
  mouse TM4SF5_reverse
cctttcccacattcctcaga

SEQ ID NO: 111 - RNA, 21 nt, Artificial
  SOCS3 (NM_174466) sense
caacaucucugucggaagauu

SEQ ID NO: 112 - RNA, 21 nt, Artificial
  SOCS3 (NM_174466) antisense
ucuuccgacagagauguuguu